(19) United States
(12) Patent Application Publication
(10) Pub. No.: US 2013/0086705 A1
(43) Pub. Date: Apr. 4, 2013

(54) COMPOSITIONS AND METHODS FOR REDUCING AND DETECTING VIRAL INFECTION

(71) Applicants: Michael David, San Diego, CA (US); Manqing Li, San Diego, CA (US)

(72) Inventors: Michael David, San Diego, CA (US); Manqing Li, San Diego, CA (US)

(73) Assignee: The Regents of the University of California

(21) Appl. No.: 13/624,153

(22) Filed: Sep. 21, 2012

Related U.S. Application Data
(60) Provisional application No. 61/540,738, filed on Sep. 29, 2011.

(51) Int. Cl. C12N 5/07

(52) U.S. Cl. CPC C12N 5/06
USPC 800/11; 514/44 A; 435/7.9; 435/5; 435/6.11; 435/455; 435/325

(57) ABSTRACT

The invention provides methods and compositions for screening candidate compounds for activity in altering the level of expression of Schlafen (Slfn) in cells, tissues and animals. The invention also provides methods and compositions for screening candidate compounds for activity in altering the level of virus infection. The invention further provides methods and compositions for screening candidate compounds for altering the level of expression of virus proteins without substantially unfaltering the level of expression of cellular proteins. The invention additionally provides methods and compositions for diagnosing the risk and/or susceptibility to virus infection. The invention also provides transgenic non-human animals, and transgenic cells, comprising a mutant Schlafen (Slfn) gene, such as a knockout mutation. These invention's compositions and methods are useful as models for virus infection, and for screening candidate prophylactic and/or therapeutic anti-viral compounds.

### CROSS-REFERENCE TO RELATED APPLICATIONS

This application claims priority under 35 U.S.C. §119(e) to co-pending U.S. Provisional Application Ser. No. 61/540,738 filed on Sep. 29, 2011, herein incorporated by reference in its entirety.

### GOVERNMENT INTEREST

This invention was made with government support under grant numbers AI047182, AI81019, AI074967, P01AI090935, R01GM101982, R21AI088490, P30AI36214, and P01AI090935 awarded by the National Institutes for Health (NIH). The government has certain rights in the invention.

### FIELD OF THE INVENTION

The invention provides methods and compositions for screening candidate compounds for activity in altering the level of expression of Schlafen (Slfn) in cells, tissues and animals. The invention also provides methods and compositions for screening candidate compounds for activity in altering the level of virus infection. The invention further provides methods and compositions for screening candidate compounds for altering the level of expression of virus proteins without substantially altering the level of expression of cellular proteins. The invention additionally provides methods and compositions for diagnosing the risk and/or susceptibility to virus infection. The invention also provides transgenic non-human animals, and transgenic cells, comprising a mutant Schlafen (Slfn) gene, such as a knockout mutation. These invention's compositions and methods are useful as models for virus infection, and for screening candidate prophylactic and/or therapeutic anti-viral compounds.

### SUMMARY OF THE INVENTION

The invention provides a method for reducing release of a retrovirus from a cell infected with the retrovirus, comprising administering to the infected cell a composition comprising an amount of a Slfn protein, or portion of the Slfn protein, wherein the amount is effective in reducing release of the retrovirus. In one embodiment, the Slfn protein comprises mammalian Slfn11 protein. In a particular embodiment, the mammalian Slfn11 protein comprises human slfn11 protein. In another embodiment, the amount does not substantially reduce release of a DNA virus. In a further embodiment, the DNA virus is selected from adenoassociated virus (AAV) and Herpes Simplex virus (HSV).

The invention also provides a method for reducing viral infection in a subject in need thereof comprising administering to the subject a therapeutically effective amount of a composition comprising Slfn protein, or portion of the Slfn protein.

Also provided by the invention is a method for detecting retrovirus infection in a cell comprising detecting Slfn produced by the cell.

The invention provides methods and compositions for screening candidate compounds for activity in altering the level of expression of Schlafen (Slfn) in cells, tissues and animals. The invention also provides methods and compositions for screening candidate compounds for activity in altering the level of virus infection. The invention further provides methods and compositions for screening candidate compounds for altering the level of expression of virus proteins without substantially altering the level of expression of cellular proteins. The invention additionally provides methods and compositions for diagnosing the risk and/or susceptibility to virus infection. The invention also provides transgenic non-human animals, and transgenic cells, comprising a mutant Schlafen (Slfn) gene, such as a knockout mutation. These invention's compositions and methods are useful as models for virus infection, and for screening candidate prophylactic and/or therapeutic anti-viral compounds.

In one embodiment, the invention provides a method for screening one or more candidate compounds for activity in altering the level of expression of Schlafen (Slfn) in a mammalian target cell, comprising a) contacting the one or more candidate compounds with a mammalian target cell that expresses Schlafen (Slfn) to produce a contacted cell, and b) measuring the level of expression of one or more Slfn in the contacted cell and the uncontacted target cell. In a particular embodiment, an altered level of expression of one or more Slfn in the contacted cell compared to in the uncontacted target cell identifies the one or more candidate compounds as having activity in altering the level of expression of Slfn in the uncontacted target cell.

The invention further provides a method for screening one or more one or more candidate compounds for activity in altering the level of virus infection of a mammalian host cell, comprising a) providing i) a virus, ii) a mammalian host cell that is susceptible to the virus, and iii) the one or more candidate compounds, b) contacting the mammalian host cell with the virus and with the one or more candidate compounds under conditions for infection of the mammalian host cell with the virus, to produce a test contacted cell, c) contacting the mammalian host cell with the virus, in the absence of the one or more candidate compounds, under conditions for infection of the transgenic mammalian cell with the virus, to produce a control contacted cell, and d) measuring the level of virus infection of the test contacted cell and of the control contacted host cell. In one embodiment, an altered level of infection of the contacted test cell compared to the control contacted cell identifies the one or more candidate compounds as having activity in altering the level of virus infection of the host cell. In particular embodiments, the method comprises detecting a reduction of from 20% to 100% in the level of the virus infection of the test contacted cell compared to the control contacted cell. In alternative embodiments, the method further comprises measuring the level of expression of one or more Slfn in one or more of the host cell, the test contacted cell, and the control contacted cell.

Also provided by the invention is a method for screening one or more candidate compounds for altering the level of expression of one or more virus proteins, comprising a) providing i) a first cell that expresses a first heterologous nucleotide sequence encoding a heterologous polypeptide, wherein the first heterologous nucleotide sequence is optimized for gene expression based on codon bias of the virus, ii) a second cell that expresses a second heterologous nucleotide sequence encoding the heterologous polypeptide, wherein the second heterologous nucleotide sequence is optimized for gene expression based on codon bias of a cell that is permissive to the virus, and iii) the one or more candidate compounds, b) contacting the one or more candidate compounds with the first cell to produce a first contacted cell, c) contacting the one or more candidate compounds with the second cell to produce a second contacted cell, and d) measuring the level of expression of the heterologous polypeptide in the first contacted cell and in the second contacted cell. In one embodiment, measuring an altered level of expression of the heterologous polypeptide in the first contacted cell and measuring a substantially unaltered level of expression of the heterologous polypeptide in the second contacted cell identifies the one or more candidate compounds as specifically altering the level of expression of one or more proteins of the virus. In another embodiment, the one or more candidate compounds comprise a test polypeptide sequence that is encoded by a nucleic acid sequence comprised in the first cell and in the second cell, and the contacting step comprises expressing the test polypeptide sequence by the first cell and by the second cell. In a further embodiment, the test polypeptide sequence comprises Schlafen (Slfn).

The invention additionally provides a method for identifying one or more Schlafen (Slfn) polymorphisms as altering the level of risk of a subject to infection by a virus, comprising a) providing i) a first sample comprising genomic Schlafen (Slfn) sequence from a first population of subjects infected by the virus, and ii) a second sample comprising genomic Schlafen (Slfn) sequence from a second population of subjects not infected by the virus, b) determining the presence of i) wild type Schlafen (Slfn) gene sequence in the first sample and in the second sample, and ii) one or more Schlafen (Slfn) polymorphisms in the first sample and in the second sample, c) measuring the ratio of the one or more Schlafen (Slfn) polymorphisms relative to the wild type Schlafen (Slfn) gene sequence in i) the first sample, and ii) in the second sample, wherein measuring a difference in the ratio in the first sample relative to the ratio in the second sample identifies the one or more Schlafen (Slfn) polymorphisms as altering the level of risk of the mammalian subject to infection by the virus.

Also provided herein is a method for determining the risk of a mammalian subject to infection by a virus, comprising detecting, in a biological sample that comprises genomic Schlafen (Slfn) sequence from the subject, one or more Schlafen (Slfn) polymorphisms that is identified by any one or more of the methods described herein. In a particular embodiment, the detecting step comprises detecting an increased ratio of the one or more Schlafen (Slfn) polymorphisms in the subject.

The invention additionally provides a method for identifying one or more Schlafen (Slfn) as altering the level of risk of a subject to infection by a virus, comprising measuring the level of expression of one or more Schlafen (Slfn) in i) a first sample from a first population of subjects infected by the virus, and ii) a second sample from a second population of subjects not infected by the virus, wherein measuring an altered level of expression in the first sample relative to the second sample, identifies the one or more Schlafen (Slfn) as altering the level of risk of the subject to infection by the virus.

Also provided by the invention is a method for determining the risk of a mammalian subject to infection by a virus, comprising detecting in a biological sample from the subject an altered level of expression of one or more Schlafen (Slfn) identified by any one or more of the methods described herein.

The invention additionally provides a method for altering the level of expression of one or more virus protein by a target mammalian cell that is susceptible to a virus, comprising administering to the cell one or more compounds in an amount that alters the level of expression of one or more Schlafen (Slfn) in the cell. In one embodiment, the method comprises measuring the level of expression of the one or more Schlafen (Slfn) in the cell. In another embodiment, the one or more compounds that alter the level of expression of the Slfn in the target mammalian cell is identified by any one or more of the methods described herein. In a particular embodiment, the cell is in vivo in a mammalian subject. In a preferred embodiment, the method further comprises measuring the level of expression of the one or more virus proteins.

Also provided by the invention is a transgenic non-human mammal comprising a homozygous deletion of at least a portion of one or more Schlafen (Slfn) gene, wherein the deletion is comprised in one or more of CD4+ T cell, macrophage cell, and dendritic cell of the mammal. In one embodiment, the at least a portion encodes from amino acid 1 to 579 of a Slfn protein. In another embodiment, the at least a portion encodes a polypeptide sequence that is at least 50-amino acids long and that is comprised by the sequence from amino acid 1 to 579 of a Slfn protein. In a further embodiment, the transgenic non-human mammal is susceptible to infection by human immunodeficiency virus (HIV). In another embodiment, the transgenic non-human mammal exhibits one or more symptoms of Acquired Immune Deficiency Syndrome (AIDS).

The invention further provides a method for screening one or more one or more candidate compounds for activity in altering the level of infection of a mammalian host cell by human immunodeficiency virus (HIV), comprising a) providing i) human immunodeficiency virus (HIV), ii) any one or more of the transgenic non-human mammals described herein, and iii) the one or more candidate compounds, b) contacting the transgenic non-human mammal with the HIV and with the one or more candidate compounds under conditions for infection of cells of the mammal with the HIV to produce a contacted test mammal, c) contacting the transgenic non-human mammal with the HIV, in the absence of the one or more candidate compounds, under conditions for infection of cells of the mammal with the HIV to produce a contacted control mammal, and d) measuring the level of HIV infection of cells of the contacted test mammal and of cells of the contacted control mammal, wherein measuring an altered level of HIV infection of the cells of the contacted test mammal compared to the cells of the contacted control mammal identifies the one or more candidate compounds as having activity in altering the level of the HIV infection of the host cell.

Also provided by the invention is a transgenic non-human mammalian cell comprising a deletion of Schlafen (Slfn) gene.

The invention further provides a method for screening one or more one or more candidate compounds for activity in altering the level of infection of a mammalian cell by a virus, comprising a) providing i) virus, ii) one or more of the transgenic mammalian cells described herein, and iii) the one or more candidate compounds, b) contacting the transgenic mammalian cell with the virus and with the one or more candidate compounds under conditions for infection of the transgenic mammalian cell with the virus to produce a contacted test cell, c) contacting the transgenic mammalian cell with the virus, in the absence of the one or more candidate compounds, under conditions for infection of the transgenic mammalian cell with the virus to produce a contacted control cell, and d) measuring the level of infection of the contacted test cell and of the contacted control cell with the virus, wherein measuring an altered level of virus infection of the contacted test cell compared to the contacted control cell identifies the one or more candidate compounds as having activity in altering the level of infection of the mammalian cell by the virus. In one embodiment, the one or more candidate compounds are identified by any one or more of the methods described herein.

### BRIEF DESCRIPTION OF THE DRAWINGS

FIG. 1: Domain structure of murine Slfn proteins.

FIG. 2: huSlfn11 inhibits retrovirus production: HEK293T cells were co-transfected with plasmids encoding V5-tagged huSlfns (wt or mutants) or the CAT control, pCL-Eco retrovirus packaging plasmid (Gag, Pol, Env) and MIG (MSCV-IRES-GFP) plasmid. The supernatant was collected and used to infect NIH3T3 cells at serial dilutions. 48 hours after infection, the cells were harvested and the number of GFP⁺ NIH 3T3 cells was determined by FACS.

FIG. 3: huSlfn11 inhibits retrovirus production: HEK293; HEK293-Slfn11^{shRNA}, HEK293-Control^{shRNA}, or HEK293T cells were transfected with MSCV-IRES-GFP plasmid and the pCL-Eco helper virus construct to produce mouse-tropic retrovirus. Virus containing culture supernatants were collected and used to infect NIH3T3 cells. Cells were harvested 48 h after infection and the number of GFP⁺ NIH3T3 cells was determined by FACS. It should be noted that neither plasmid contained an SV40 origin of replication.

FIG. 4: huSlfn11 inhibits HIV production: HEK293; HEK293-Slfn11^{shRNA}, HEK293-Control^{shRNA}, or HEK293T cells were co-transfected with pCMV-VSV-g Env and pNL43LucR+E− HIV plasmids (see below) and pcDNA5-GFP vector. The supernatants were collected, and virus titers were determined by luciferase assays of target cells infected by serially diluted supernatants. The titer was normalized to the number of GFP⁺ virus-producing cells to account for minor differences in transfection efficiency between the cell lines.

FIG. 5: huSlfn11 does not significantly affect intracellular vRNA levels: The indicated cells were co-transfected with pNL43LucR+E− HIV and pcDNA5-GFP plasmid (no pCMV-VSV-g Env), after 48 hrs cells were collected and total RNA was extracted. The amount of viral RNA was determined by qPCR with primers for the luciferase sequence. Viral RNA levels were normalized to the number of GFP⁺ cells to account for minor variations (<2%) in transfection efficiency between cells.

FIG. 6: hSlfn11 does not inhibit infection by retrovirus: VSVg-pseudotyped HIV carrying luciferase (see FIG. 5) was isolated from virus producing cells, and used to infect HEK293; HEK293-Slfn11^{shRNA}, HEK293-Control^{shRNA}, or HEK293T cells. The cells were collected 48 hrs post transfection, and luciferase activity was determined (normalized to total protein).

FIG. 7: huSlfn11 selectively inhibits viral protein expression based on codon usage preference. A) 293T cells were transfected with HIV-1 pNL4-3.Luc.R⁺E⁻ vector and pcDNA5 EGFP together with plasmids encoding either huSlfn5, huSlfn11 or CAT as indicated. The expression of the indicated HIV-derived proteins and of EGFP and GAPDH were detected by immunoblotting of whole cell lysates. B) Top panels: 293T cells were co-transfected with HIV-1 pNL4-3.Luc.R⁺E⁻ vector and either huSlfn5, huSlfn11 or CAT as indicated (left panels), or 293, 293shRNA^{Ctl} and 293shRNA^{Slfn} cells were transfected with HIV-1 pNL4-3.Luc.R⁺E⁻ vector (right panels). Total cell lysates were probed for the presence of luciferase and GAPDH by immunoblotting. Bottom panels: Same as above, except pNL4-3-DEnv-EGFP was used instead of pNL4-3.Luc.R⁺E⁻, and the resulting lysates were immunoblotted for Nef and GAPDH.

FIG. 8: huSlfn11 selectively inhibits viral protein expression based on codon usage. Indicated cells were transfected with V5-tagged viral codon usage-based gag (Gag^{vir}, top) or synonymous human codon optimized gag (Gag^{opt}, bottom). Expression levels of Gag were determined by anti-V5 immunoblotting.

FIG. 9: huSlfn11 inhibits viral expression based on codon usage. A) 293shRNA^{Ctl} and 293shRNA^{Slfn} cells were transfected with V5-tagged GFP or EGFP vectors with or without HIV-1 pNL4-3.Luc.R⁺E⁻ vector, and the expression of (E)GFP and GAPDH was detected by immunoblotting B) To account for minor differences (<2 fold) in (E)GFP mRNA levels, (E)GFP proteins and mRNAs were quantitated from three experiments as outlined above, and GFP and EGFP protein levels were normalized to their respective mRNAs.

FIG. 10: The AAA+ motifs of Slfn family members.

FIG. 11: huSlfn11 binds tRNA: Total tRNA was ³²P end-labeled, and used as probe in EMSAs utilizing decreasing amounts (3.5, 0.7 or 0.15 μg) FPLC-purified 6×His-huSlfn11N, or 6×His-GFP.

FIG. 12: HIV/huSlfn11 change tRNA pool: Total tRNA from 293shRNA^{Ctl} and 293shRNA^{Slfn} with or without pNL4-3.Luc.R⁺E⁻ vector was analyzed by tRNA-array (1).

FIG. 13: huSlfn11 inhibits HIV replication. A) HumanPBMCs were treated with 1,000 U/ml IFNβ, and Slfn11 expression analyzed by immunoblotting B) huSlfn11 levels in CEM, CEMshRNA^{Ctl} and CEMshRNA^{Slfn} cells were analyzed by immunoblotting with anti-huSlfn11 antibodies. C) CEM, CEMshRNA^{Ctl} and CEMshRNA^{Slfn} cells were infected with HIV-1_{LAI} at an MOI of 0.01. The extent of viral replication was determined by HIV p24 ELISA of culture supernatants at indicated time points.

FIG. 14-huSlfn11 inhibits retrovirus production without affecting intracellular vRNA levels. 293T cells were transfected with pNL4-3.Luc.R⁺E⁻/pCMV-VSV-G together with huSlfn5, huSlfn11, huSlfn11N, huSlfn11C or CAT (a, c, e, g), or 293, 293shRNA^{Ctl}, 293shRNA^{Slfn} and 293T cells were transfected with pNL4-3.Luc.R⁺E⁻ and pCMV-VSV-G (b, d, f, h). a, b) VSV-G-pseudotyped HIV production was assayed by titrated infection and luciferase assay; c, d) Viral particle content in supernatants was analyzed by p24 ELISA; e) and f) extracellular vRNA concentration was analyzed by qPCR of p24; g) and h) intracellular vRNA was determined by qPCR of p24. (avg+/−s.d.; n=3)

FIG. 15—huSlfn11 selectively inhibits viral protein expression based on codon usage. a) 293T cells were transfected with pNL4-3.Luc.R⁺E⁻ and pcDNA5-EGFP together with huSlfn5, huSlfn11 or CAT, and cell lysates immunoblotted for HIV proteins, EGFP and GAPDH; b) 293, 293shRNA^{Ctl} and 293shRNA^{Slfn} cells were transfected with pNL4-3.Luc.R⁺E⁻ and pcDNA5-EGFP, and lysates analyzed as in a); c) Top: 293T cells were co-transfected with pNL4-3.Luc.R⁺E⁻ and huSlfn5, huSlfn11 or CAT (left), or 293, 293shRNA^{Ctl} and 293shRNA^{Slfn} cells were transfected with pNL4-3.Luc.R⁺E⁻ (right). Lysates were probed for luciferase and GAPDH. Bottom: as above, except pNL4-3-DEnv-EGFP was used instead of pNL4-3.Luc.R⁺E⁻, and lysates immunoblotted for Nef and GAPDH; d) Cells were transfected with viral codon usage-based gag (Gag^{vir}, top) or synonymous human codon usage-optimized gag (Gag^{opt}, bottom). Expression of Gag in cell lysates was determined by anti-V5 immunoblotting.

FIG. 16-huSlfn11 binds tRNAs and selectively inhibits protein expression based on codon usage. a) 293shRNA^{Ctl} and 293shRNA^{Slfn} cells were transfected with pNL4-3.Luc.R⁺E⁻ or control vector (indicated as +/−HIV). Relative abundances of mature tRNA species were analyzed by microarray as described^{25,26}; b) Left: increasing amounts of huSlfn11N or GFP were incubated with ³²P-labelled tRNA and subject to EMSA. Right: 2× or 10× unlabeled tRNA or in-vitro transcribed vRNA corresponding to the gag-pol frame-shifting sequence (120b) were added to the binding reaction; c) As in b), except non-specific (NS) or anti-huSlfn11 (□S11) monoclonal antibody was added to the binding reaction; d) 293T cells were transfected with V5-tagged GFP, Myc-tagged EGFP and pNL4-3.Luc.R⁺E⁻ together with either CAT, huSlfn5, or huSlfn11 (left). Lysates were probed for V5-GFP, Myc-EGFP and GAPDH. Similarly, V5-tagged GFP, Myc-tagged EGFP and pNL4-3.Luc.R⁺E⁻ were co-transfected into 293, 293shRNA^{Ctl} and 293shRNA^{Slfn} cells (right), and V5-GFP, Myc-EGFP and GAPDH expression determined by immunoblotting. e) V5-GFP and Myc-EGFP protein levels of d) were quantitated, and normalized to V5-GFP and Myc-EGFP mRNA levels, respectively (avg+/−s.d.; n=4).

FIG. 17-huSlfn11 inhibits replication of wild-type HIV-1_{LAI} in CEM cells. a) Human PBMCs were stimulated with 6,000 U/ml IFNβ. huSlfn11 expression in the derived lysates and in CEM cell lysates was analyzed by immunoblotting. b) huSlfn11 expression in CEM, CEMshRNA^{Ctl} and CEMshRNA^{Slfn} cells as analyzed by immunoblotting; c) CEM, CEMshRNA^{Ctl} and CEMshRNA^{Slfn} cells were infected with HIV-1_{LAI} at an MOI of 0.01, and viral replication was assayed by p24 ELISA of culture supernatants (avg+/−s.d.; n=4).

FIG. 18—Relative expression and inducibility of huSlfns. a) Human HFF cells were stimulated with IFNβ (5000 U/μl), pdAdT (1 μg/ml) or pC (1 pg/ml) for 6 hours, and levels of the indicated mRNAs and TBP were determined by qPCR. The expression of the Slfn and ISG54 mRNAs are presented as ΔCt between TBP and the Slfn mRNA of interest. b) Total cellular RNA was prepared from the indicated cell lines, and mRNA levels of the huSlfns and of TBP were quantitated by qPCR and presented as in a). c) huSlfn11 mRNA levels in 293, 293shRNACtl, 293shRNASlfn and 293T cells as determined by qPCR; d) Same as c), except huSlfn11 protein was visualized by immunoblotting of whole cell lysates.

FIG. 19—huSlfn11 does not inhibit early viral infection steps. a) 293, 293shRNACtl, 293shRNASlfn and 293T cells were infected with pseudotyped HIV-1VSV-G virus at the indicated dilutions, and infection efficiency was determined by the expression levels of the transduced luciferase in the infected cells.

FIG. 20—huSlfn11 inhibits production of MSCV but not AAV. a) 293, 293shRNACtl, 293shRNASlfn and 293T cells were transfected with MSCV-IRES-GFP retrovirus vector and pCL-Eco ecotropic retrovirus packaging vector. Viral titers in the supernatants were determined by infection and subsequent flow-cytometric analysis of GFP expression of NIH3T3 cells. b) 293T cells were transfected with MSCV-IRES-GFP retrovirus vector/pCL-Eco packaging vector and plasmids encoding either huSlfn5, huSlfn11, huSlfn11N, huSlfn11C or CAT as indicated. Virus production was assayed by titrated infection of NIH3T3 cells as in a). c) 293, 293shRNACtl, 293shRNASlfn and 293T cells were transfected with pXX6, pXX2 and pACLALuc, and rAAVLuc production determined by luciferase assay following titrated infection.

FIG. 21—huSlfn11 does not alter cytoplasmic vRNA levels or cause accumulation of viral particles. a) 293T cells were transfected with pNL4-3.Luc.R+E− together with either huSlfn5, huSlfn11 or CAT. After cellular fractionation, cytoplasmic vRNA concentration was analyzed by qPCR; b) 293T cells were transfected with HIV-1 pNL4-3.Luc.R+E− vector together with pcDNA6-CAT or pcDNA6-Slfn11. 48 hours later, the cells were fixed and images were acquired by electronic microscopy.

FIG. 22—huSlfn11 inhibition is independent of viral enzymes or nuclear export of unspliced RNA. a) 293T cells were co-transfected with HIV-1 pNL4-3.Luc.R+E−PRstop vector and either huSlfn5, huSlfn11 or CAT as indicated (left panels), or 293, 293shRNACtl and 293shRNASlfn cells were transfected with pNL4-3.Luc.R+E−PRstop vector (right panels). Whole cell lysates derived 48 hours after transfection were immunoblotted with anti-Gag and anti-GAPDH antibodies. b) 293T cells were transfected with psPAX2 (encoding Gag, Pol, Tat & Rev without any LTR elements) and pNL-GFP-RRE(SA) (providing spliceable RNA yielding GFP only if exported as unspliced RNA via Rev/RRE interaction) together with plasmids encoding either huSlfn5, huSlfn11, huSlfn11N, huSlfn11C or CAT as indicated. Whole cell lysates were immunoblotted with anti-GFP, anti-Gag and anti-GAPDH antibodies.

FIG. 23—Non-discriminating tRNA-binding of huSlfn11. Gel purified 32P-labeled total tRNA was incubated with increasing amounts of huSlfn11N to obtain approximate 50% shifting on a native gel. 32P-labeled tRNA from shifted and unshifted bands was recovered and samples hybridized on microarrays containing 96 probes (probes are repeated 8 times each and are complementary to human/mouse nuclear-encoded and mitochondrial-encoded tRNAs).

FIG. 24—huSlfn11 inhibits HIV replication. The pLKO.1 shRNA vector against Slfn11 (TRCN0000152057, CCGGCAGTCTTTGAGAGAGCTTATTCT-CGAGAATAAGCTCTCTCAAAGACTGTTTTTTG) was used to independently create a second huSlfn11-deficient CEM cell line (CEMshRNASlfn in this figure). CEM, CEMshRNACtl and the new CEMshRNASlfn cells were infected with HIV-1LAI at an MOI of 0.01. The extent of viral replication was determined by HIV p24 ELISA of culture supernatants at indicated time points.

FIG. 25—Amino acid sequence of exemplary Slfn proteins listed in Table 1.

### DEFINITIONS

To facilitate understanding of the invention, a number of terms are defined below.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed using a recombinant DNA molecule.

The term “recombinant mutation” refers to a mutation that is introduced by means of molecular biological techniques. This is in contrast to mutations that occur in nature.

The terms “endogenous” and “wild type” when in reference to a sequence refer to a sequence which is naturally found in the cell or virus into which it is introduced so long as it does not contain some modification relative to the naturally-occurring sequence. The term “heterologous” refers to a sequence that is not endogenous to the cell or virus into which it is introduced.

The terms “mutation” and “modification” refer to a deletion, insertion, or substitution.

A “deletion” is defined as a change in a nucleic acid sequence or amino acid sequence in which one or more nucleotides or amino acids, respectively, is absent.

An “insertion” or “addition” is that change in a nucleic acid sequence or amino acid sequence that has resulted in the addition of one or more nucleotides or amino acids, respectively.

A “substitution” in a nucleic acid sequence or an amino acid sequence results from the replacement of one or more nucleotides or amino acids, respectively, by a molecule that is a different molecule from the replaced one or more nucleotides or amino acids. For example, a nucleic acid may be replaced by a different nucleic acid as exemplified by replacement of a thymine by a cytosine, adenine, guanine, or uridine. Alternatively, a nucleic acid may be replaced by a modified nucleic acid as exemplified by replacement of a thymine by thymine glycol. Substitution of an amino acid may be conservative or non-conservative. “Conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine may be conservatively substituted one for the other since each is a basic, charged (hydrophilic) amino acid. “Non-conservative substitution” is a substitution other than a conservative substitution. Guidance in determining which and how many amino acid residues may be substituted, inserted or deleted without abolishing biological and/or immunological activity may be found using computer programs well known in the art, for example, DNAStar™ software.

A “variant” or “homolog” of an amino acid sequence of interest or nucleotide sequence of interest refers to a sequence that has at least 99% identity and/or at least 95% identity and/or at least 90% identity and/or at least 85% identity and/or at least 80% identity and/or at least 75% identity and/or at least 70% identity and/or at least 65% identity with the an amino acid sequence of interest or nucleotide sequence of interest.

The term “expression vector” as used herein refers to a recombinant DNA molecule containing a desired coding sequence and appropriate nucleic acid sequences necessary for the expression (e.g., transcription and/or translation) of the operably linked coding sequence in a particular host organism. Expression vectors are exemplified by, but not limited to, plasmid, phagemid, shuttle vector, cosmid, virus, chromosome, mitochondrial DNA, plastid DNA, and nucleic acid fragment. Nucleic acid sequences used for expression in prokaryotes include a promoter, optionally an operator sequence, a ribosome binding site and possibly other sequences. Eukaryotic cells are known to utilize promoters, enhancers, and termination and polyadenylation signals.

“Purify” and grammatical equivalents thereof when in reference to a desirable component (such as cell, protein, nucleic acid sequence, carbohydrate, etc.) refer to the reduction in the amount of at least one undesirable component (such as cell, protein, nucleic acid sequence, carbohydrate, etc.) from a sample, including a reduction by any numerical percentage of from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100%. Thus purification results in “enrichment” (i.e., an increase) in the amount of the desirable component relative to one or more undesirable component.

“Isolated” and grammatical equivalents thereof when in reference to a desirable component (such as cell, protein, nucleic acid sequence, carbohydrate, etc.) refer to a component that is chemically cleaved from a native element, molecule, or structure and/or is chemically synthesized such that the “isolated” component is one that does not exist as in nature, and/or has a distinctive chemical identity from that of the native element, molecule, or structure to make the component markedly different from the one that exists in nature.

The terms “operable combination” and “operably linked” when in reference to the relationship between nucleic acid sequences and/or amino acid sequences refers to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest resulting in an mRNA that directs the synthesis of a polypeptide encoded by the nucleotide sequence of interest. The term also refers to the linkage of amino acid sequences in such a manner so that a functional protein is produced.

The term “infection” of a cell by a virus refers to the level of the virus at any one or more stage of its life cycle (e.g., viral fusion with the cell to gain entry, replication of virus genomic DNA and/or RNA, transcription of virus genomic DNA into RNA, translation of genomic sequences into virus proteins, assembly of virus proteins into virus particles, and/or release of virus particles from the cell), as determined, directly or indirectly, by any method. For example, cell infection by HIV may be determined by, without limitation, in vitro cell-cell fusion assays using the exemplary HeLa-P5L and HeLa-ADA cell lines, in vitro HIV infection assays using peripheral blood mononuclear cells (PMBC), and/or in vivo HIV infection assays in animals, such as humanized mouse models and macaque models.

A “susceptible” cell refers to a cell that is capable of being infected by a virus. “Infection” refers to adsorption of the virus to the cell and penetration into the cell. Susceptible cells include permissive and non-permissive cells. Susceptibility of a cell to a virus may be determined by detecting the presence of virus proteins and/or virus RNA and/or virus DNA in extracts of infected cells.

A “permissive” cell refers to a cell that is both susceptible to a virus and capable of supporting replication of viral nucleic acid sequences and/or viral peptide sequences. While not required, in one embodiment, a cell is permissive if the replicated viral nucleic acid sequences and viral peptide sequences are assembled into a virion particle. In some embodiments, a permissive cell releases the assembled virions contained therein. In other embodiments, the assembled virions remain inside the permissive cells without release. Viral replication may be determined by, for example, production of viral nucleic acid sequences and/or of viral peptide sequences. Production of progeny virus may be determined by observation of a cytopathic effect. However, this method is less preferred than detection of virus nucleotide sequences and/or virus protein sequences, since a cytopathic effect may not be observed even when viral replication is detectable by detecting virus nucleotide sequences and/or virus protein sequences.

A cell that is “not permissive” (i.e., “non-infections”) is a cell that is not capable of supporting viral replication.

The terms “immortalized cell” and “cell line” interchangeably refer to a cell that is capable of a greater number of cell divisions in vitro before cessation of proliferation and/or senescence as compared to a primary cell from the same source. Cell lines may be produced by passaging of cells using methods known in the art. Briefly, a confluent or subconfluent population of cells which is adhered to a solid substrate (e.g., plastic Petri dish) is released from the substrate (e.g., by enzymatic digestion), and a proportion (e.g., 10%) of the released cells is seeded onto a fresh substrate. The cells are allowed to adhere to the substrate, and to proliferate in the presence of appropriate culture medium. The ability of adhered cells to proliferate may be determined visually by observing increased coverage of the solid substrate over a period of time by the adhered cells. Alternatively, proliferation of adhered cells may be determined by maintaining the initially adhered cells on the solid support over a period of time, removing and counting the adhered cells and observing an increase in the number of maintained adhered cells as compared to the number of initially adhered cells. In some embodiments, the cell line is capable of at least 10, more preferably at least 50, and most preferably at least 100, cell divisions prior to senescence.

The term “transgenic” when used in reference to a cell refers to a cell which contains a transgene and/or whose genome has been manipulated by man by molecular biological techniques. Transgenic cells may be produced by several methods including the introduction of a “transgene” comprising nucleic acid (usually DNA) into a target cell or integration of the transgene into a chromosome of a target cell by way of human intervention, such as by the methods described herein.

The term “transgene” as used herein refers to any nucleic acid sequence which is introduced into the cell by experimental manipulations. A transgene may be an “endogenous DNA sequence” or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally-occurring sequence. The term “heterologous DNA sequence” refers to a nucleotide sequence which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes an endogenous DNA sequence which contains some modification. Generally, although not necessarily, heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

“Wild-type” when in reference to a cell, molecule, etc., refers to a cell, molecule, etc. whose structure is the same as found in nature without alteration by man (such as by chemical and/or molecular biological techniques, etc.).

“Polymorphism” when in reference to a genomic sequence refers to differences in the sequence of the genomic sequence between individuals, wherein the differences are too common to be due merely to new mutation. In preferred embodiments, the polymorphism has a frequency of at least 1% in the population.

“Optimize for gene expression based on codon bias of a virus” when in reference to a nucleotide sequence encoding a protein, means increasing the proportion the nucleotide sequence's codons that are preferentially and/or specifically used by the virus when compared to another virus and/or compared to a cell. “Optimize for gene expression based on codon bias of a cell” when in reference to a nucleotide sequence encoding a protein, means increasing the proportion the nucleotide sequence's codons that are preferentially and/or specifically used by the cell when compared to another cell and/or compared to a virus. Methods of optimizing gene expression based on codon bias of a virus and/or cell are known in the art (U.S. Pat. Nos. 6,096,304 and 8,129,119; US 2007/0042047).

“Reporter” and “marker” are used interchangeably to refer to a DNA, RNA, and/or polypeptide sequence that is detectable in any detection system, including, but not limited to enzyme (e.g., ELISA, as well as enzyme-based histochemical assays), fluorescent, and luminescent systems. Exemplary reporter genes include, for example, β-glucuronidase gene, green fluorescent protein (GFP) gene, E. coli β-galactosidase (LacZ) gene, Halobacterium β-galactosidase gene, Neuropsora tyrosinase gene, human placental alkaline phosphatase gene, and chloramphenicol acetyltransferase (CAT) gene, Aequorin (jellyfish bioluminescence) gene, Firefly luciferase (EC 1.13.12.7) from the American firefly, Photinus pyralis, Renilla luciferase (EC 1.13.12.5) from the sea pansy Renilla reniformis, and Bacterial luciferase (EC 1.14.14.3) from Photobacterium fischeri.

The terms “posttranscriptional gene silencing” and “PTGS” refer to silencing of gene expression in plants after transcription, and appears to involve the specific degradation of mRNAs synthesized from gene repeats. The term “cosuppression” refers to silencing of endogenous genes by heterologous genes that share sequence identity with endogenous genes.

“Mammalian cell” refers to a cell of a mammal.

“Mammal” includes human, non-human primate, murine (such as mouse and rat), ovine, bovine, ruminant, lagomorph, porcine, caprine, equine, canine, feline, and ave. In particular embodiments, a mammalian cell is exemplified by a cell from mouse, rat, guinea pig, hamster, ferret and chinchilla.

A subject “in need” of a particular intervention, such as in need of reducing one or more symptoms of a disease and/or of altering the level of expression of a protein and/or of altering the level of infection by a pathogen, includes a subject that exhibits and/or is at risk of exhibiting one or more symptoms for which intervention may be desired. For Example, subjects may be at risk based on family history, genetic factors, environmental factors, etc. This term includes animal models of the disease. Thus, administering a composition to a subject in need of reducing one or more symptoms of a disease and/or of altering the level of expression of a protein and/or of altering the level of infection by a pathogen, includes prophylactic administration of the composition (i.e., before symptoms are clinically and/or or sub-clinically detectable) and/or therapeutic administration of the composition (i.e., after symptoms are clinically and/or or sub-clinically detectable).

The term “specifically” altering the level of expression of a first protein (such as a virus protein, or a protein that is encoded by a first nucleotide sequence that is optimized for gene expression based on codon bias of a virus) is a relative term that means that the quantity of the first protein and/or the quantity of mRNA encoding the first protein is altered (i.e., increased or decreased by any statistically significant amount) without substantially altering the level of expression of a second reference protein (such as a mammalian cell protein, or a protein encoded by a second nucleotide sequence that is optimized for gene expression based on codon bias of a cell that is permissive to a virus)

“Biological sample” includes specimens obtained from a subject, including body fluids (such as urine, blood, plasma, fecal matter, cerebrospinal fluid (CSF), semen, sputum, and saliva), as well as solid tissue. Biological samples also include a cell (such as cell lines, cells isolated from tissue whether or not the isolated cells are cultured after isolation from tissue, fixed cells such as cells fixed for histological and/or immunohistochemical analysis), tissue (such as biopsy material), cell extract, tissue extract, and nucleic acid (e.g., DNA and RNA) isolated from a cell and/or tissue, and the like.

A “control sample” refers to a sample used for comparing to another sample by maintaining the same conditions in the control and other samples, except in one or more particular variable in order to infer a causal significance of this varied one or more variable on a phenomenon. For example, a “positive control sample” is a control sample in which the phenomenon is expected to occur. For example, a “negative control sample” is a control sample in which the phenomenon is not expected to occur.

The terms “reduce,” “inhibit,” “diminish,” “suppress,” “decrease,” and grammatical equivalents (including “lower,” “smaller,” etc.) when in reference to the level of any molecule (e.g., amino acid sequence, and nucleic acid sequence, antibody, etc.), cell, virus particle, and/or phenomenon (e.g., level of expression of a protein (such as Schlafen (Slfn) and/or virus protein), level of virus infection of a cell, risk to disease, disease symptom, binding to a molecule, specificity of binding of two molecules, affinity of binding of two molecules, specificity to disease, sensitivity to disease, affinity of binding, enzyme activity, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first subject is exemplified by, but not limited to, a subject that has been manipulated using the invention's compositions and/or methods. In a further embodiment, the second subject is exemplified by, but not limited to, a subject that has not been manipulated using the invention's compositions and/or methods. In an alternative embodiment, the second subject is exemplified by, but not limited to, a subject to that has been manipulated, using the invention's compositions and/or methods, at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions and/or methods is sought to be determined in one individual. In another embodiment, the first and second subjects may be different individuals, such as when comparing the effect of the invention's compositions and/or methods on one individual participating in a clinical trial and another individual in a hospital.

The term “not substantially reduced” when in reference to the level of any molecule (e.g., amino acid sequence, and nucleic acid sequence, antibody, etc.), cell, virus particle, and/or phenomenon (e.g., level of expression of a protein (such as Schlafen (Slfn) and/or virus protein), level of virus infection of a cell, risk to disease, disease symptom, binding to a molecule, specificity of binding of two molecules, affinity of binding of two molecules, specificity to disease, sensitivity to disease, affinity of binding, enzyme activity, etc.) in a first sample (or in a first subject) relative to a second sample (or relative to a second subject), means that the quantity of molecule, cell and/or phenomenon in the first sample (or in the first subject) is from 91% to 100% of the quantity in the second sample (or in the second subject).

The terms “increase,” “elevate,” “raise,” and grammatical equivalents (including “higher,” “greater,” etc.) when in reference to the level of any molecule (e.g., amino acid sequence, and nucleic acid sequence, antibody, etc.), cell, virus particle, and/or phenomenon (e.g., level of expression of a protein (such as Schlafen (Slfn) and/or virus protein), level of virus infection of a cell, risk to disease, disease symptom, binding to a molecule, specificity of binding of two molecules, affinity of binding of two molecules, specificity to disease, sensitivity to disease, affinity of binding, enzyme activity, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first subject is exemplified by, but not limited to, a subject that has been manipulated using the invention's compositions and/or methods. In a further embodiment, the second subject is exemplified by, but not limited to, a subject that has not been manipulated using the invention's compositions and/or methods. In an alternative embodiment, the second subject is exemplified by, but not limited to, a subject to that has been manipulated, using the invention's compositions and/or methods, at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions and/or methods is sought to be determined in one individual. In another embodiment, the first and second subjects may be different individuals, such as when comparing the effect of the invention's compositions and/or methods on one individual participating in a clinical trial and another individual in a hospital.

The terms “alter” and “modify” when in reference to the level of any molecule (e.g., amino acid sequence, and nucleic acid sequence, antibody, etc.), cell, virus particle, and/or phenomenon (e.g., level of expression of a protein (such as Schlafen (Slfn) and/or virus protein), level of virus infection of a cell, risk to disease, disease symptom, binding to a molecule, specificity of binding of two molecules, affinity of binding of two molecules, specificity to disease, sensitivity to disease, affinity of binding, enzyme activity, etc.) refer to an increase or decrease.

“Without substantially altering,” “substantially unaltered” and grammatical equivalents when in reference to the level of any molecule (e.g., amino acid sequence, and nucleic acid sequence, antibody, etc.), cell, virus particle, and/or phenomenon (e.g., level of expression of a protein (such as Schlafen (Slfn) and/or virus protein), level of virus infection of a cell, risk to disease, disease symptom, binding to a molecule, specificity of binding of two molecules, affinity of binding of two molecules, specificity to disease, sensitivity to disease, affinity of binding, enzyme activity, etc.) means that the quantity of molecule, cell and/or phenomenon in the first sample (or in the first subject) is not increased or decreased by a statistically significant amount relative to the second sample (or in a second subject). Thus in one embodiment, the quantity of molecule, cell and/or phenomenon in the first sample (or in the first subject) is from 90% to 100% (including, for example, from 91% to 100%, from 92% to 100%, from 93% to 100%, from 94% to 100%, from 95% to 100%, from 96% to 100%, from 97% to 100%, from 98% to 100%, and/or from 99% to 100%) of the quantity in the second sample (or in the second subject).

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

### BRIEF DESCRIPTION OF THE INVENTION

The inventors have identified a family of interferon-induced proteins (schlafen=Slfn) that act as viral restriction factors. Specifically, the inventors demonstrate that Slfn11 expression inhibits the production of (but not infection by) retroviruses. Surprisingly, abrogation of Slfn11 expression substantially increases the release of retrovirus by producing cells.

Slfn proteins were first identified in 1998 by Steve Hedrick, however, their function remained elusive. The inventors found Slfn proteins induced by interferon regulatory factor 3 (IRF3) and interferons after pathogen recognition, and have been working towards identifying their cellular function since 2003. So far, no prior art reports indicate the antiviral properties of Slfn proteins.

The invention's Slfn11 protein inhibits the production of retrovirus from infected cells, rather than preventing the initial infection. The inventors hypothesized that other Slfn family members will exert a similar effect on other virus groups. The inventors have identified the point of action in the (selective) inhibition of viral protein synthesis and consequential reduction in the assembly of the viral particle.

The invention provides compounds or proteins that increase Slfn expression or activation would present a novel group of anti-(retro)viral drugs.

The invention provides methods for monitoring Slfn expression that are useful prognostic and/or diagnostic indicator of symptom development (e.g. AIDS) as a consequence of viral infection.

For example, data herein show that huSlfn11 potently and specifically abrogates the production of retroviruses such as HIV-1. The studies revealed that huSlfn11 has no effect on the early steps of the retroviral infection cycle including reverse transcription, integration and transcription. Rather, huSlfn11 acts at the late stage of virus production by selectively inhibiting the expression of viral proteins in a codon usage-dependent manner. We further find that huSlfn11 binds tRNA, and counteracts changes in the tRNA pool elicited by the presence of HIV. In summary, our studies identified a novel antiviral mechanism within the innate immune response in which huSlfn11 selectively inhibits viral protein synthesis in HIV-infected cells by means of codon-bias discrimination.

Furthermore, data herein show that analysis of the HIV replication cycle revealed that huSlfn11 does not inhibit reverse transcription, integration, or production and nuclear export of vRNA, nor did we observe a block in budding or release of viral particles. However, we discovered a selective inhibition in the synthesis of virally encoded proteins. Intriguingly, Coccia et al. had previously observed a specific inhibition of viral protein synthesis in HIV-infected cells in response to interferon, but did not identify the factors responsible for the effect (Coccia et al., J Biol Chem 269, 23087-23094 (1994)). While not intending to limit the invention to any particular mechanism, the inventors postulate that, in one embodiment, huSlfn11 acts at the point of virus protein synthesis by exploiting the distinct viral codon bias towards A/T nucleotides. This model supports the notion that the antiviral activity of huSlfn11 extends to other viruses with rare codon bias such as influenza, but not to AAV or HSV. While not intending to limit the invention to any particular mechanism, in one embodiment, huSlfn11 interacts with all tRNAs in-vitro. Direct binding of huSlfn11 to tRNA offers the possibility that huSlfn11 either sequesters tRNAs, prevents their maturation via post-transcriptional processing or accelerates their deacylation. In either case, if already rare tRNAs are further reduced, tRNA availability might manifest as the rate limiting step in the synthesis of proteins involving those tRNAs. In contrast, a lesser or no impact would be expected on proteins synthesized via plentiful tRNAs, as even if a fraction of those tRNAs is ‘eliminated’, translation initiation will likely remain the rate limiting event. In conclusion, our results establish huSlfn11 as a potent, interferon-inducible restriction factor against retroviruses such as HIV, mediating its antiviral effects on the basis of codon usage discrimination.

1. Methods for Screening Compounds that Alter Schlafen Expression

The invention provides a method for screening one or more candidate compounds for activity in altering (i.e., increasing or decreasing) the level of expression of Schlafen (Slfn) in a mammalian target cell, comprising a) contacting the one or more candidate compounds with a mammalian target cell that expresses Schlafen (Slfn) (e.g., expresses Slfn mRNA and/or protein) to produce a contacted cell, and b) measuring the level of expression of one or more Slfn (e.g., level of Slfn mRNA and/or protein) in the contacted cell. Thus, in one embodiment, an altered level of expression of one or more Slfn in the contacted cell compared to in the target cell identifies the one or more candidate compounds as having activity in altering (i.e., increasing or decreasing) the level of expression of Slfn in the target cell.

The invention's methods are useful in, for example, identifying compounds that alter the antiviral activity of Slfn in cells, and that may be used to increase and/or decrease the level of one or more virus proteins that are formed inside the cell and/or released from the cell, and/or used to increase and/or decrease the number of virus particles formed inside the cell, and/or used to increase and/or decrease the number of virus particles released from the cell.

In one embodiment, the method may further comprise measuring the level of expression of the one or more Slfn (e.g., level of Slfn mRNA and/or protein) in one or more of the target cell and the contacted cell.

In some embodiment, the invention contemplates that the step of measuring the level of expression of one or more Slfn comprises one or more of measuring the level of Slfn mRNA and measuring the level of Slfn protein.

The invention further provides a compound identified using any one or more of the methods and/or compositions described herein. In one embodiment, the compound is purified and/or isolated.

The invention also provides a compound identified the invention's methods. In one embodiment, the compound is purified and/or isolated.

2. Methods for Screening Compounds that Alter Virus Infection

The invention further provides a method for screening one or more one or more candidate compounds for activity in altering (i.e., increasing or decreasing) the level of virus infection of a mammalian host cell, comprising a) providing i) a virus, ii) a mammalian host cell that is susceptible to the virus, and iii) one or more candidate compounds, b) contacting the mammalian host cell with the virus and with the one or more candidate compounds to produce a contacted cell, and c) measuring the level of virus infection of the contacted cell (e.g., level of virus genomic DNA and/or RNA, level of transcription of virus genomic DNA into RNA, level of one or more virus proteins, number of virus particles formed inside the cell and/or number of virus particles released from the cell). In one embodiment, an altered level of infection of the contacted cell compared to a cell that is not contacted with the one or more candidate compounds identifies the one or more candidate compounds as having activity in altering (i.e., increasing or decreasing) the level of virus infection of the host cell.

In one embodiment, one or more candidate compounds are identified by one or more of the methods described herein.

The invention contemplates that the virus, mammalian host cell that is susceptible to the virus, and the one or more candidate compounds are contacted in any order. Thus, in one embodiment, the step of contacting comprises contacting the host cell with the virus prior to contacting with the one or more candidate compounds. This is useful in, for example, identifying prophylactic compounds that may be used before exposure to, and/or before infection with, a virus.)

In another embodiment, the step of contacting comprises contacting the host cell with the virus after contacting with the one or more candidate compounds. This is useful in, for example, identifying therapeutic compounds that alter one or more steps of viral infection after exposure to, and/or after infection with, a virus, including the level of viral fusion with the cell membrane to gain entry into the cell, level of virus genomic DNA and/or RNA, level of transcription of virus genomic DNA into RNA, level of one or more virus proteins, number of virus particles formed inside the cell, and/or number of virus particles released from the cell.

In a further embodiment, the step of contacting comprises contacting the host cell substantially simultaneously with the virus and with the one or more candidate compounds. This is useful in, for example, identifying prophylactic and therapeutic compounds that alter one or more steps of viral infection before exposure to, and/or before infection with, after exposure to, and/or after cell infection with a virus (e.g., level of virus genomic DNA and/or RNA, level of transcription of virus genomic DNA into RNA, level of one or more virus proteins, number of virus particles formed inside the cell, and/or number of virus particles released from the cell)

In some embodiments, it may be desirable to detecting a reduction in the level of the virus infection of the mammalian host cell. In some embodiments, the reduction is from 20% to 100% in the level of the virus infection of the mammalian host cell, including any percentage reduction there between, such as from 25% to 100%, from 30% to 100%, from 35% to 100%, from 40% to 100%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, and from 95% to 100%.

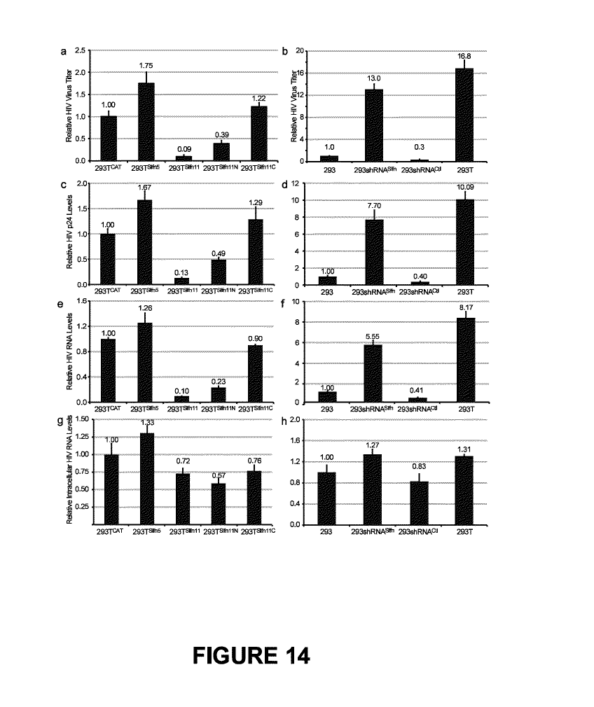

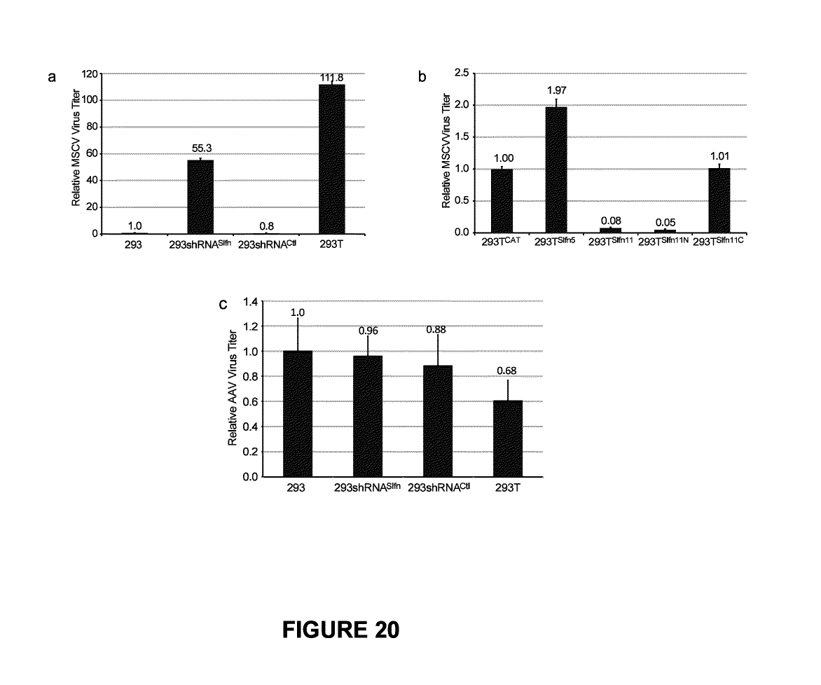

For example, data herein demonstrate that huSlfn11 inhibited HIVVSV-G (FIG. 14a) production from 293T cells from a control titer of 1.0 to a titer of 0.09 (i.e., 91% inhibition), and also inhibited MSCV (FIG. 20b) production from 293T cells from a control titer of 1.0 to a titer of 0.08 (i.e., 92% inhibition).

Also, data herein also demonstrate that the AAA-domain-containing, NH2-terminal region (huSlfn11-N; amino acid 1-579) inhibited HIVVSV-G (FIG. 14a) production from 293T cells from a control titer of 1.0 to a titer of 0.39 (i.e., 61% inhibition), and also inhibited MSCV (FIG. 20b) production from 293T cells from a control titer of 1.0 to a titer of 0.05 (i.e., 95% inhibition).

In some embodiment, the step of measuring the level of virus infection of the contacted cell comprises measuring one or more of the a) level of one or more genomic nucleic acid sequences of the virus, b) level of virus RNA that is transcribed from one or more genomic DNA sequence of the virus, c) level of one or more virus proteins, d) number of virus particles formed inside the contacted cell, and e) number of virus particles released from the contacted cell.

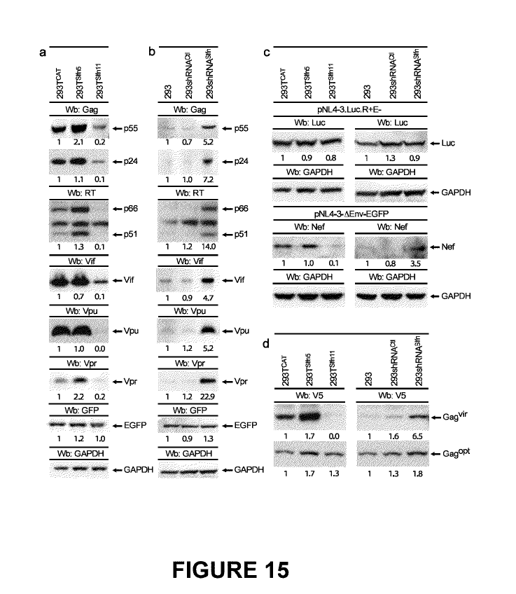

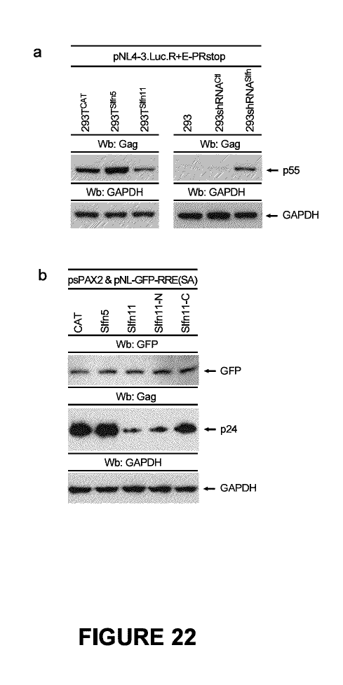

For example, where the virus comprises a retrovirus, such as Human Immunodeficiency Virus (HIV), the step of measuring the level of virus infection may include measuring the level of viral RNA encoding, and/or measuring the level of one or more HIV proteins, exemplified by, but not limited to p55 group specific antigen (Gag), p24 capsid protein, Reverse Transcriptase (RT) protein, Viral Infectivity Factor (Vif) protein, Viral Protein U (Vpu) protein, and Viral Protein R (Vpr) protein. Data herein demonstrate that huSlfn11 inhibited expression of HIV p55 Gag protein, p24 capsid protein (FIGS. 15a, 15b, and 22), RT protein, Vif protein, Vpu protein and Vpr protein (FIGS. 15a and b).

In some embodiments, the methods may further comprise measuring the level of expression of one or more Slfn (e.g., level of Slfn mRNA and/or protein) in one or more of the host cell and the contacted cell.

The invention also provides a compound identified the invention's methods. In one embodiment, the compound is purified and/or isolated.

3. Methods for Screening Compounds that Differentially Alter Viral Protein Synthesis Compared to Cellular Protein Synthesis

The invention also provides a method for screening one or more candidate compounds for altering (i.e., increasing or decreasing) the level of expression of one or more virus proteins, comprising a) providing i) a first cell that expresses a first heterologous nucleotide sequence encoding a heterologous polypeptide, wherein the first heterologous nucleotide sequence is optimized for gene expression based on codon bias of the virus, ii) a second cell that expresses a second heterologous nucleotide sequence encoding the heterologous polypeptide, wherein the second heterologous nucleotide sequence is optimized for gene expression based on codon bias of a cell that is permissive to the virus, and iii) one or more candidate compounds, b) contacting the one or more candidate compounds with the first cell to produce a first contacted cell, c) contacting the one or more candidate compounds with the second cell to produce a second contacted cell, and d) measuring the level of expression (e.g., level of mRNA and/or protein) of the heterologous polypeptide in the first contacted cell and in the second contacted cell. In one embodiment, an altered level of expression of the heterologous polypeptide in the first contacted cell and a substantially unaltered level of expression of the heterologous polypeptide in the second contacted cell identifies the one or more candidate compounds as specifically altering (i.e., increasing or decreasing) the level of expression of one or more proteins of the virus.

The invention's method is useful for identifying compounds that alter the level of infection of a cell by a virus (e.g., translation of virus genomic sequences into virus proteins, assembly of virus proteins into virus particles, and/or release of virus particles from the cell) without substantially altering the level of cellular protein expression by cells harboring the virus.

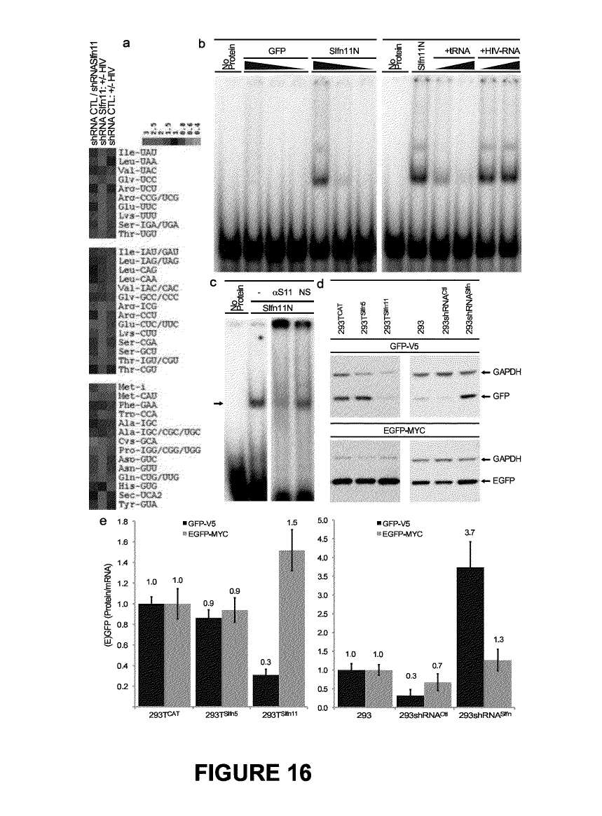

In one embodiment, the one or more candidate compounds comprise a test polypeptide sequence that is encoded by a nucleic acid sequence comprised in the first cell and in the second cell, and the contacting step comprises expressing the test polypeptide sequence by the first cell and by the second cell. Thus, in a particular embodiment, the test polypeptide sequence comprises Schlafen (Slfn). Data herein demonstrate the effect of the exemplary heterologous test polypeptide sequence huSlfn11 on expression of Gag protein (Example 11, FIG. 15d) and on expression of GFP (Example 14, FIGS. 16d and e) that are encoded by DNA sequences that are optimized for gene expression based on codon bias of a virus or of a mammalian cell.

In a further embodiment, the heterologous polypeptide comprises a marker protein (such as green fluorescent protein (GFP)). Data herein (Example 14, FIGS. 16d and e) demonstrate that the exemplary protein huSlfn11 inhibited the expression of GFP (which harbors similar codon-bias as HIV), whereas expression of EGFP (which has been optimized for gene expression based on codon bias of mammalian cells), was largely refractory to inhibition by huSlfn11.

In another embodiment, the heterologous polypeptide comprises a virus protein. Data herein (Example 11, FIG. 15d) demonstrate that the exemplary protein huSlfn11 strongly altered expression of Gag protein that was optimized for gene expression based on codon bias of HIV (Gag^{vir}), but did not substantially alter expression of Gag protein that was optimized for gene expression based on codon bias of human cells (Gag^{opt}).

In a further embodiment, the heterologous polypeptide comprises a marker protein. Data herein (Example 14, FIGS. 16d and e) demonstrate that the exemplary protein huSlfn11 inhibited the expression of GFP (which harbors similar codon-bias as HIV), whereas expression of EGFP (which has been optimized for gene expression based on codon bias of mammalian cells), was largely refractory to inhibition by huSlfn11.

### 4. Methods for Determining Risk of Viral Infection

a. Schlafen (Slfn) Polymorphism

The invention provides methods for determining whether one or more Schlafen (Slfn) polymorphisms are associated with increased risk for disease (e.g., infection with virus). Thus, in one embodiment, the invention provides a method for identifying one or more Schlafen (Slfn) polymorphisms as altering the level of risk of a subject to infection by a virus, comprising a) providing i) a first sample (e.g. cells, tissues, cell extracts, tissue extracts, etc.) comprising genomic Schlafen (Slfn) sequence from a first population of subjects infected by the virus, and ii) a second (e.g., control) sample comprising genomic Schlafen (Slfn) sequence from a second population of subjects not infected by the virus, b) determining the presence of one or more of i) wild type Schlafen (Slfn) gene sequence in the first sample and in the second sample, and ii) one or more Schlafen (Slfn) polymorphisms in the first sample and in the second sample, c) measuring the ratio of the one or more Schlafen (Slfn) polymorphisms relative to the wild type Schlafen (Slfn) gene sequence in one or more of i) the first sample, and ii) the second sample.

Generic methods for determining the association of genetic polymorphisms with susceptibility to disease are known in the art (Lee et. al., “Genetic polymorphisms and susceptibility to lung disease, J Negat Results Biomed. 2006; 5: 5.; Lin et al. “HLA-E gene polymorphism associated with susceptibility to Kawasaki disease and formation of coronary artery aneurysms,” Arthritis & Rheumatism Volume 60, Issue 2, pages 604-610, February 2009.

In some embodiments, the method further comprises detecting a difference in the ratio in the first sample relative to the ratio in the second sample. In one embodiment, a difference identifies the one or more Schlafen (Slfn) polymorphisms as altering the level of risk of the mammalian subject to infection by the virus.

The invention further provides methods for determining the risk of disease (e.g., infection by a virus) based on the presence of one or more Schlafen (Slfn) polymorphisms. Thus, in one embodiment, the invention provides a method for determining the risk of a mammalian subject to infection by a virus, comprising detecting, in a biological sample that comprises genomic Schlafen (Slfn) sequence from the subject, one or more Schlafen (Slfn) polymorphisms. In one embodiment, one or more Schlafen (Slfn) polymorphisms are identified by any of the methods described herein. In a further embodiment, the detecting step optionally comprises detecting an increased ratio of the one or more Schlafen (Slfn) polymorphisms in the subject, such as compared to a control subject lacking infection by the virus.

b. Schlafen (Slfn) Expression

The invention provides methods for determining whether an altered level of expression of Schlafen (Slfn) is associated with increased risk for disease (e.g., infection with virus). Thus, in one embodiment, the invention provides a method for identifying Schlafen (Slfn) as altering the level of risk of a subject to infection by a virus, comprising measuring the level of expression of Schlafen (Slfn) in i) a first sample (e.g. cells, tissues, cell extracts, tissue extracts, etc.) from a first population of subjects infected by the virus, and ii) a second (e.g., control) sample from a second population of subjects not infected by the virus. In one embodiment, the method further comprises detecting an altered level of expression in the first sample relative to the second sample. In a particular embodiment, detecting an altered level identifies Schlafen (Slfn) as altering the level of risk of the subject to infection by the virus.

The invention further provides methods for determining the risk of disease (e.g., infection by a virus) based on an altered level of Schlafen (Slfn) expression. Thus, in one embodiment, the invention provides a method for determining the risk of a mammalian subject to infection by a virus, comprising detecting an altered level of expression of Schlafen (Slfn) in a biological sample from the subject (e.g., compared to a control subject lacking infection by the virus).

### 5. Methods for Altering the Levels of Virus Infection

The invention provides methods for reducing and/or increasing virus protein synthesis, and/or reducing and/or increasing the level of virus particles formed inside a cell, and/or reducing and/or increasing the level of virus particles released from a cell.

Thus, in one embodiment, the invention provides a method for altering the level of expression of virus protein (and/or the number of virus particles formed inside an infected cell and/or the number of virus particles released from an infected cell) by a target mammalian cell that is susceptible to a virus, comprising administering to the cell one or more compounds in an amount that alters (i.e., increases or decreases) the level of expression of one or more Schlafen (Slfn) in the cell. The invention's methods are useful in, for example, prophylactic and/or therapeutic applications, to prevent and/or reduce viral infection. This is also useful in applications where it is desirable to increase the production of viral proteins and/or viral particles, such as for use in the production of vaccines, in gene therapy for delivery of drugs and/or genes, etc.)

In one embodiment, the method further comprises measuring the level of expression of the one or more Schlafen (Slfn) in the cell. In one preferred embodiment, measuring the level of expression of the one or more Slfn comprises measuring one or more of the a) level of genomic nucleic acid sequences encoding the one or more Slfn, b) level of mRNA encoding the one or more Slfn, and c) level of the one or more Slfn protein.

In a particular embodiment, one or more of the compounds that alter (i.e., increase or decrease) the level of expression of the Slfn in the target mammalian cell is identified by any one or more of the methods described herein.

Thus, in some embodiments, the target cell is infected with the virus. This is useful in, for example, prophylactic and/or therapeutic applications, to prevent and/or reduce viral infection. This is also useful in applications where it is desirable to increase the production of viral proteins and/or viral particles, such as for use in the production of vaccines, in gene therapy for delivery of drugs and/or genes, etc.

In another embodiment, the target cell is not infected with the virus. This is useful in, for example, therapeutic applications, to reduce viral infection after it occurs. This is also useful in applications where it is desirable to increase the production of viral proteins and/or viral particles, such as for use in the production of vaccines, in gene therapy for delivery of drugs and/or genes, etc.

In one embodiment, the target cell is permissive to the virus, and/or is a transgenic cell that expresses one or more heterologous Slfn, and/or is a wild-type cell that expresses endogenous Slfn, and/or is a human cell, and/or is a non-human cell, and/or is a cell capable of generating a multicellular animal (e.g., fertilized egg cell, eight-cell embryo, blastocoele cell, midgestation embryo cell, and embryonic stem (ES) cell)

In particular embodiments, it may be desirable to reduce virus protein synthesis, and/or reduce the level of virus particles formed inside a cell, and/or reduce the level of virus particles released from a cell. This may be useful in, for example, prophylaxis and treatment of viral infection. Thus, in some embodiments, the cell is in vitro. In other embodiments, the cell is in vivo in a mammalian subject. In a further embodiment, the subject is in need of one or more of a) reducing the level of expression of one or more proteins of the virus, b) reducing the number of virus particles inside the subject's cells, and c) reducing the number of virus particles released from the subject's cells. In some embodiment, the method further comprises measuring the level of expression of the one or more virus proteins. In a particular embodiment, the step of measuring comprises measuring one or more of the a) level of one or more genomic nucleic acid sequences of the virus, b) level of virus RNA that is transcribed from one or more genomic DNA sequence of the virus, c) level of one or more virus proteins, d) number of virus particles formed inside cells of the subject, and e) number of virus particles released from cells of the subject.

In particularly preferred embodiments, the virus is a retrovirus that comprises Human Immunodeficiency Virus (HIV). Data herein demonstrate inhibition of HIV particle formation and/or release (FIG. 14a). In a further preferred embodiment, the HIV protein is exemplified by one or more of p55 group specific antigen (Gag), p24 capsid protein, Reverse Transcriptase (RT) protein, Viral Infectivity Factor (Vif) protein, Viral Protein U (Vpu) protein, and Viral Protein R (Vpr) protein. Data herein demonstrate that huSlfn11 inhibited expression of HIV p55 Gag protein, p24 capsid protein (FIGS. 15a, 15b, and 22), RT protein, Vif protein, Vpu protein and Vpr protein (FIGS. 15a and b).

In particular embodiments, it may be desirable to increase virus protein synthesis, and/or increase the level of virus particles formed inside a cell, and/or increase the level of virus particles released from a cell. This may be useful in, for example, vaccine applications and/or gene therapy applications.

### 6. Transgenic Animals

The invention also provides a transgenic non-human mammal comprising a homozygous deletion of at least a portion of one or more Schlafen (Slfn) gene, wherein the deletion is comprised in one or more of CD4+ T cell, macrophage cell, and dendritic cell of the mammal.

Methods for producing knockout animals are known in the art (U.S. Pat. No. 7,247,767). Knockout animals may be produced using transgenic cells that comprise a deletion of Schlafen (Slfn) gene, and that are capable of generating an animal, such as fertilized egg cell, eight-cell embryo, blastocoele cell, midgestation embryo cell, and embryonic stem (ES) cell. For example, fertilized egg cells may be implanted into the uterus of a pseudopregnant female and allowed to develop into an animal. These cells have successfully been used to produce transgenic mice, sheep, pigs, rabbits and cattle (Hammer et al., (1986) J. Animal Sci.:63:269; Hammer et al., (1985) Nature 315:680-683). Other cells include pre-implantation embryo cells. For example, blastomere cells (Jaenisch, (1976) Proc. Natl. Acad. Sci USA 73:1260-1264; (Jahner et al., (1985) Proc. Natl. Acad. Sci. USA 82:6927-6931; Van der Putten et al., (1985) Proc. Natl. Acad Sci USA 82:6148-6152), and eight-cell embryo cells from which the zona pellucida has been removed (Van der Putten (1985), supra; Stewart et al., (1987) EMBO J. 6:383-388). The pre-implantation embryos which are manipulated in accordance with the invention's methods may be transferred to foster mothers for continued development. Alternatively, cells may be at a later stage of embryonic development, such as blastocoele cells and midgestation embryo cells (Jahner et al., (1982) Nature 298:623-628). Yet another cell type is an embryonic stem (ES) cell. ES cells are pluripotent cells which may be directly derived from, for example, the inner cell mass of blastocysts (Doetchman et al., (1988) Dev. Biol. 127:224-227), from inner cell masses (Tokunaga et al., (1989) Jpn. J. Anim. Reprod. 35:173-178), from disaggregated morulae (Eistetter, (1989) Dev. Gro. Differ. 31:275-282) or from primordial germ cells (Matsui et al., (1992) Cell 70:841-847). Transgenic mice may be generated from ES cells which have been treated in accordance with the invention's methods by injection of several ES cells into the blastocoel cavity of intact blastocysts (Bradley et al., (1984) Nature 309:225-256). Alternatively, a clump of ES cells may be sandwiched between two eight-cell embryos (Bradley et al., (1987) in “Teratocarcinomas and Embryonic Stem Cells: A Practical Approach,” Ed. Robertson E. J. (IRL, Oxford, U.K.), pp. 113-151; Nagy et al., (1990) Development 110:815-821). Both methods result in germ line transmission at high frequency.)

In a particular embodiment, the transgenic non-human mammal is susceptible to infection by human immunodeficiency virus (HIV). In another embodiment, the transgenic non-human mammal exhibits one or more symptoms of Acquired Immune Deficiency Syndrome (AIDS), such as one or more of a CD4+ T cell count below 200 cells per microliter, pneumocystis pneumonia, cachexia, esophageal candidiasis, respiratory tract infections, opportunistic infections by bacteria, viruses, fungi and parasites, Kaposi's sarcoma, Burkitt's lymphoma, primary central nervous system lymphoma, cervical cancer, prolonged fevers, sweats (particularly at night), swollen lymph nodes, chills, weakness, weight loss and diarrhea. These animals are useful as an animal model for Acquired Immune Deficiency Syndrome (AIDS) that is caused by the human immunodeficiency virus (HIV). This animal model may be used in, for example, testing the efficacy of one or more candidate compounds in reducing HIV infection of one or more cells of the transgenic mammal and/or reducing one or more symptoms of AIDS in the transgenic mammal (described below).

7. Methods for Using Transgenic Animals for Screening Compounds that Alter Virus Infection

The invention contemplates using the transgenic animals for screening compounds that alter virus infection. Thus, in one embodiment, the invention provides a method for screening one or more candidate compounds for activity in altering (i.e., increasing or decreasing) the level of infection of a mammalian host cell by human immunodeficiency virus (HIV), comprising a) providing i) human immunodeficiency virus (HIV), ii) any one or more of the transgenic non-human mammals described herein (e.g., mammal comprising a homozygous deletion of Schlafen (Slfn) gene), and iii) one or more candidate compounds, b) contacting the transgenic non-human mammal with the HIV and with the one or more candidate compounds under conditions for infection of cells of the mammal with the HIV, and c) measuring the level of infection of the cells with the HIV (e.g., level of HIV genomic RNA, level of one or more HIV proteins, number of HIV particles formed inside the cell, and/or number of HIV particles released from the cell). In one embodiment, an altered level of infection of the cells compared to cells that are not contacted with the one or more candidate compounds identifies the one or more candidate compounds as having activity in altering (i.e., increasing or decreasing) the level of the HIV infection of the cells.

The invention contemplates that the virus, transgenic non-human mammal, and the one or more candidate compounds are contacted in any order. Thus, in one embodiment, the step of contacting comprises contacting the transgenic non-human mammal with the HIV prior to contacting with the one or more candidate compounds. This is useful in, for example, identifying prophylactic compounds that may be used before exposure to, and/or before infection with, HIV.)

In another embodiment, the step of contacting comprises contacting the transgenic non-human mammal with the HIV after contacting with the one or more candidate compounds. This is useful in, for example, identifying therapeutic compounds that alter one or more steps of HIV infection after exposure to, and/or after infection with, HIV, including the level of HIV fusion with the cell membrane to gain entry into the cell, level of HIV genomic RNA, level of transcription of HIV genomic RNA into protein, level of one or more HIV proteins, number of HIV particles formed inside the cell, and/or number of HIV particles released from the cell.

In a further embodiment, the step of contacting comprises contacting the transgenic non-human mammal substantially simultaneously with the HIV and with the one or more candidate compounds. This is useful in, for example, identifying prophylactic and therapeutic compounds that alter one or more steps of HIV infection before exposure to, and/or before infection with, after exposure to, and/or after cell infection with HIV (e.g., level of HIV genomic RNA, level of transcription of HIV genomic RNA into protein, level of one or more HIV proteins, number of HIV particles formed inside the cell, and/or number of HIV particles released from the cell.

In one embodiment, it may be desirable to measure the effect of the one or more candidate compounds on one or more symptoms of Immune Deficiency Syndrome (AIDS).

The invention further provides a compound identified by using any of the transgenic animals described herein. In one embodiment, the compound is purified and/or isolated.

### 8. Transgenic Cells

The invention provides a transgenic non-human mammalian cell comprising a deletion of Schlafen (Slfn) gene. These cells are useful in measuring the effect of test agents on HIV infection in these cells, and/or for producing the transgenic knockout animals of the invention, and/or for producing virus proteins for generating vaccines, and as packaging cells for the generation of virus particles in gene therapy applications.

In one embodiment, the transgenic non-human mammalian cell is derived from any one or more of the transgenic non-human mammals described herein.

In a particular embodiment, the transgenic non-human mammalian cell is susceptible to a virus. These cells are useful in, for example, producing virus proteins for generating vaccines. Additional uses include use as packaging cells for the generation of virus particles that contain therapeutic genes for gene therapy applications (e.g., cancer therapy).

In a particular embodiment, the deletion of Schlafen (Slfn) gene is a heterozygous deletion or a homozygous deletion.

In a further embodiment, the transgenic non-human mammalian cell comprises an immortalized cell.

In a particular embodiment, the transgenic non-human mammalian cell comprises one or more of CD4+ T cells, macrophage cells, and dendritic cells. These cells are useful in, for example, measuring the effect of test agents on HIV infection in these cells.

In a further embodiment, the transgenic non-human mammalian cell comprises one or more of fertilized egg cell, eight-cell embryo, blastocoele cell, midgestation embryo cell, and embryonic stem (ES) cell. These cells are useful in, for example, producing the transgenic knockout animals of the invention.

9. Methods for Using Transgenic Cell Lines for Screening Compounds that Alter Virus Infection

The invention contemplates using the transgenic cell lines for screening compounds that alter virus infection. Thus, in one embodiment, the invention provides a method for screening one or more one or more candidate compounds for activity in altering (i.e., increasing or decreasing) the level of infection of a mammalian cell by a virus (such as HIV), comprising a) providing i) virus (e.g., HIV), ii) any one or more of the transgenic mammalian cells described herein (e.g., transgenic cell comprising a deletion of Schlafen (Slfn) gene), and iii) one or more candidate compounds, b) contacting the transgenic mammalian cell with the virus and with the one or more candidate compounds under conditions for infection of the transgenic mammalian cell with the virus, and c) measuring the level of infection of the cells with the virus (e.g., level of virus genomic DNA and/or RNA, level of one or more virus proteins, number of virus particles formed inside the cell, and/or number of virus particles released from the cell). In one embodiment, an altered level of infection of the cells compared to cells that are not contacted with the one or more candidate compounds identifies the one or more candidate compounds as having activity in altering (i.e., increasing or decreasing) the level of the virus infection of the cells.

The invention contemplates that the virus, transgenic mammalian cell, and the one or more candidate compounds are contacted in any order. Thus, in one embodiment, the step of contacting comprises contacting the transgenic mammalian cell with the virus prior to contacting with the one or more candidate compounds. This is useful in, for example, identifying prophylactic compounds that may be used before exposure to, and/or before infection with, virus.)

In another embodiment, the step of contacting comprises contacting the transgenic mammalian cell with the virus after contacting with the one or more candidate compounds. This is useful in, for example, identifying therapeutic compounds that alter one or more steps of virus infection after exposure to, and/or after infection with, virus, including the level of virus fusion with the cell membrane to gain entry into the cell, level of virus genomic RNA and/or DNA, level of transcription of virus genomic sequences into protein, level of one or more virus proteins, number of virus particles formed inside the cell, and/or number of virus particles released from the cell.

In a further embodiment, the step of contacting comprises contacting the transgenic mammalian cell substantially simultaneously with the virus and with the one or more candidate compounds. This is useful in, for example, identifying prophylactic and therapeutic compounds that alter one or more steps of virus infection before exposure to, and/or before infection with, after exposure to, and/or after cell infection with virus (e.g., level of virus genomic sequences, level of transcription of virus genomic sequences into protein, level of one or more virus proteins, number of virus particles formed inside the cell, and/or number of virus particles released from the cell.

The invention further provides a compound identified by using any of the transgenic cells described herein. In one embodiment, the compound is purified and/or isolated.

### 10. Schlafen (Slfn)

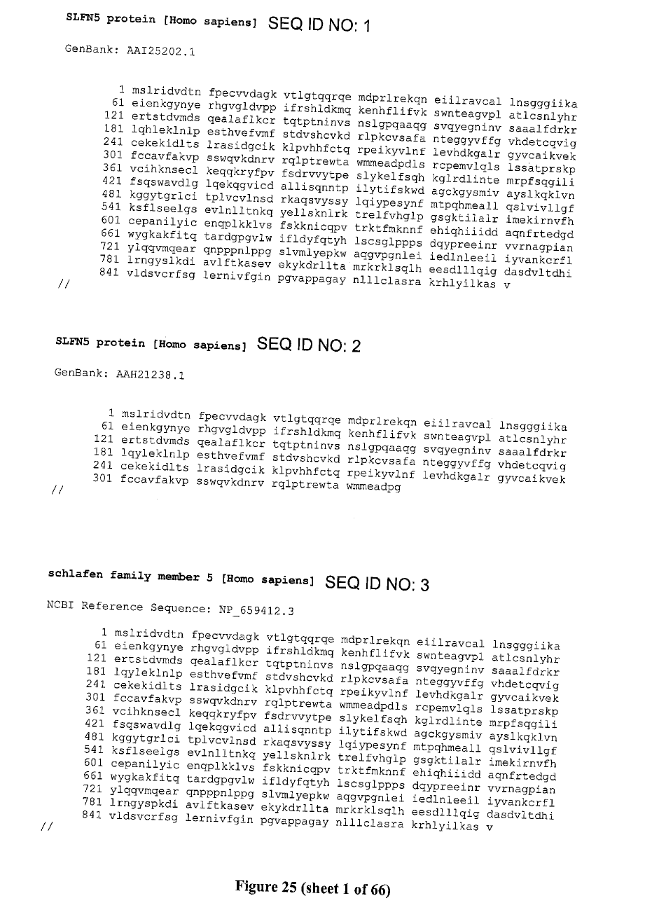

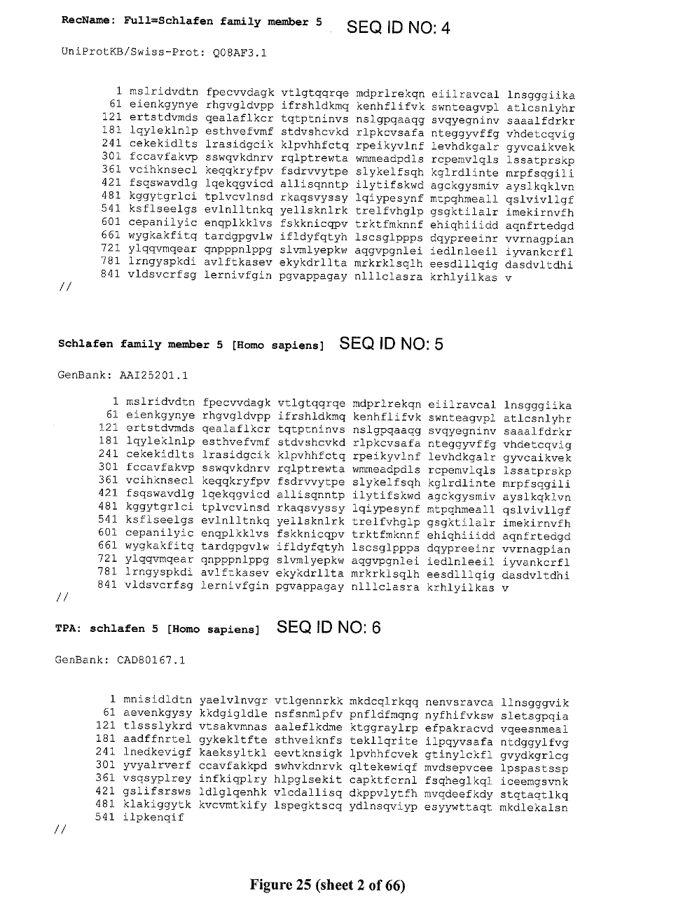

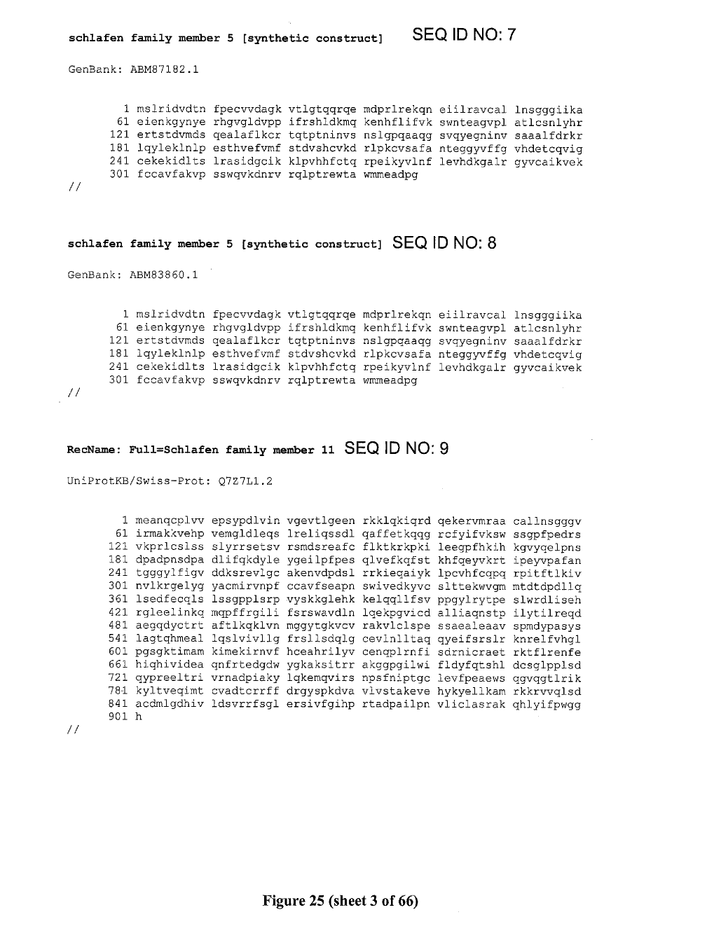

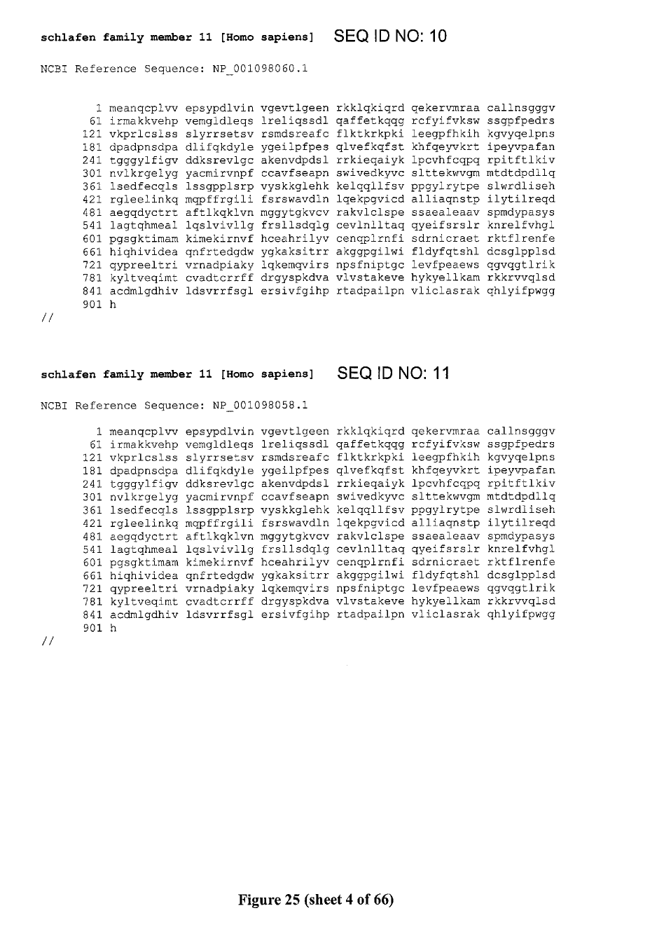

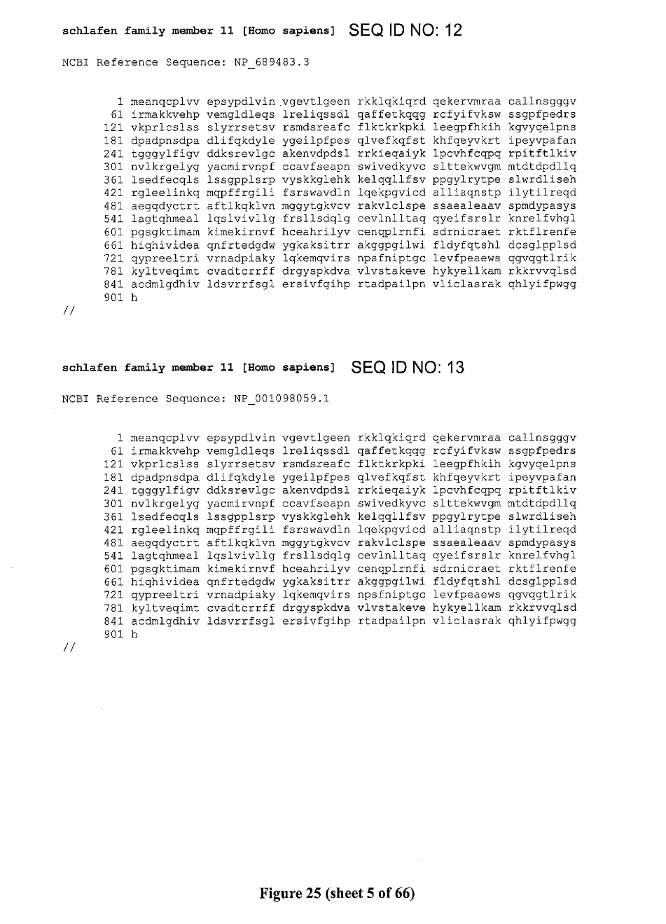

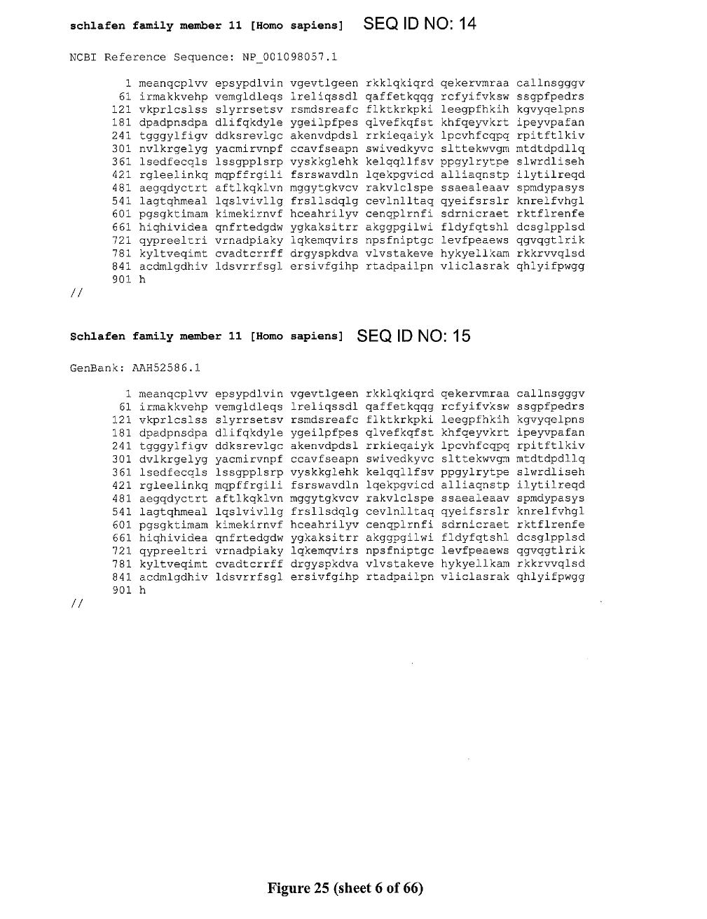

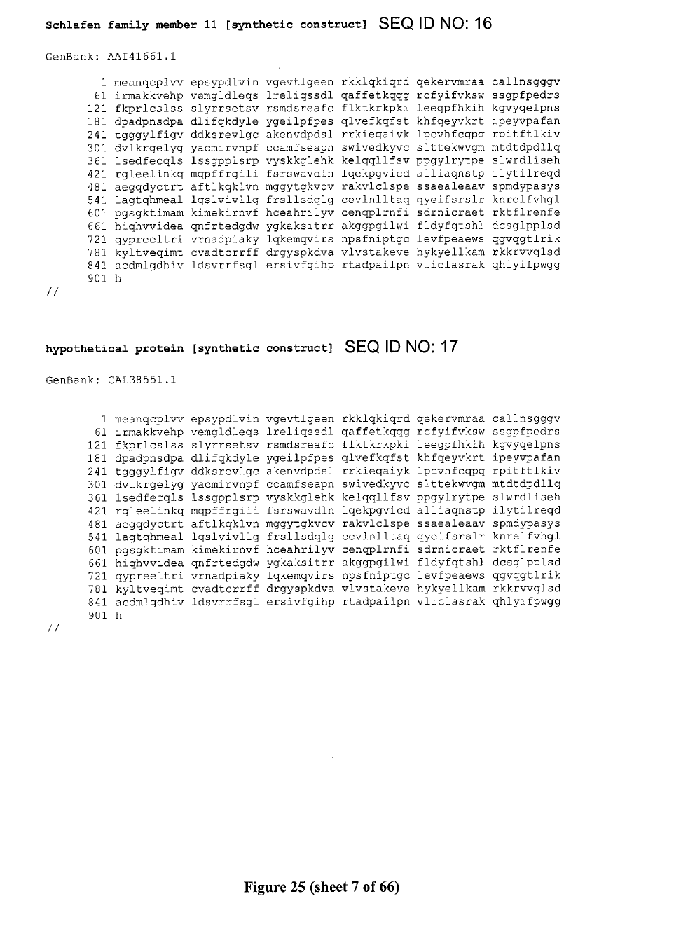

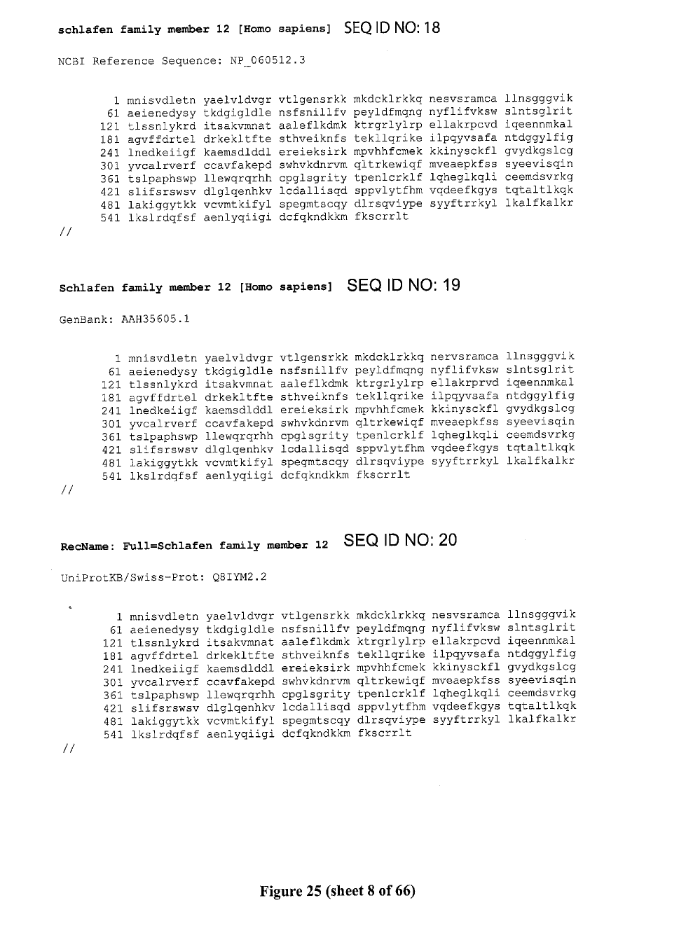

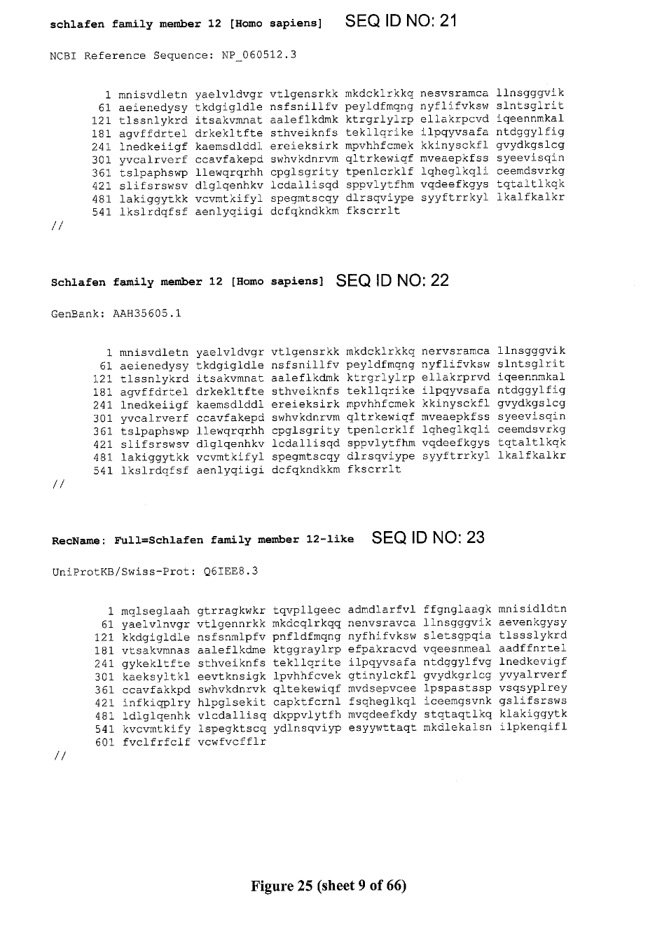

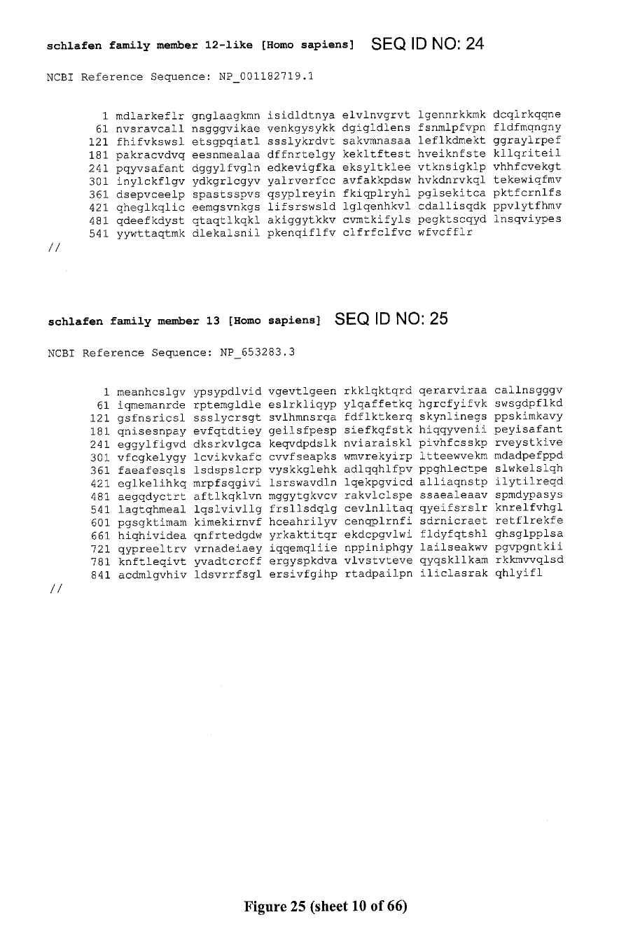

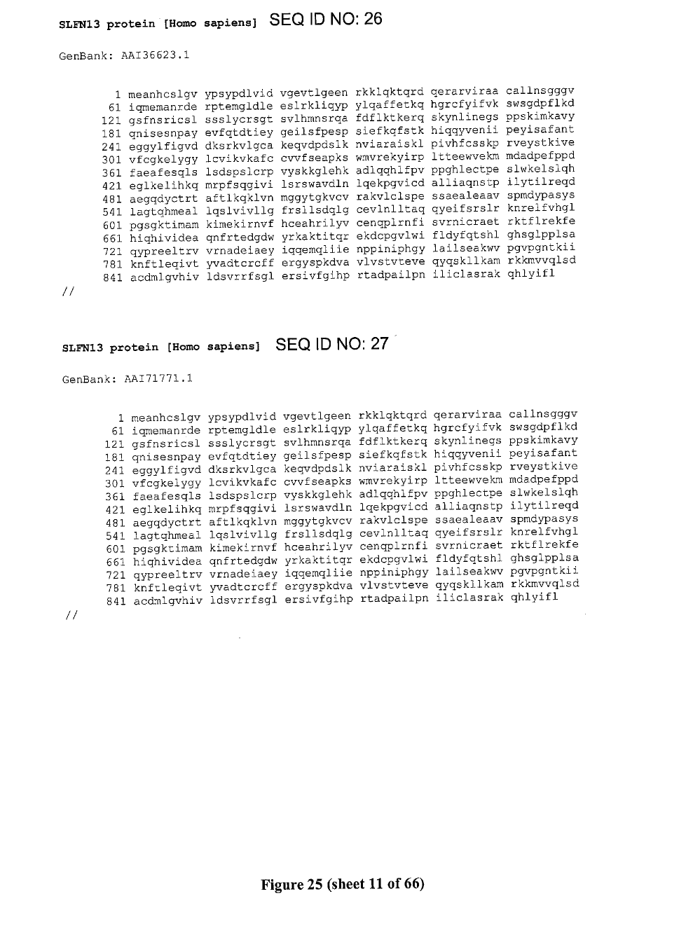

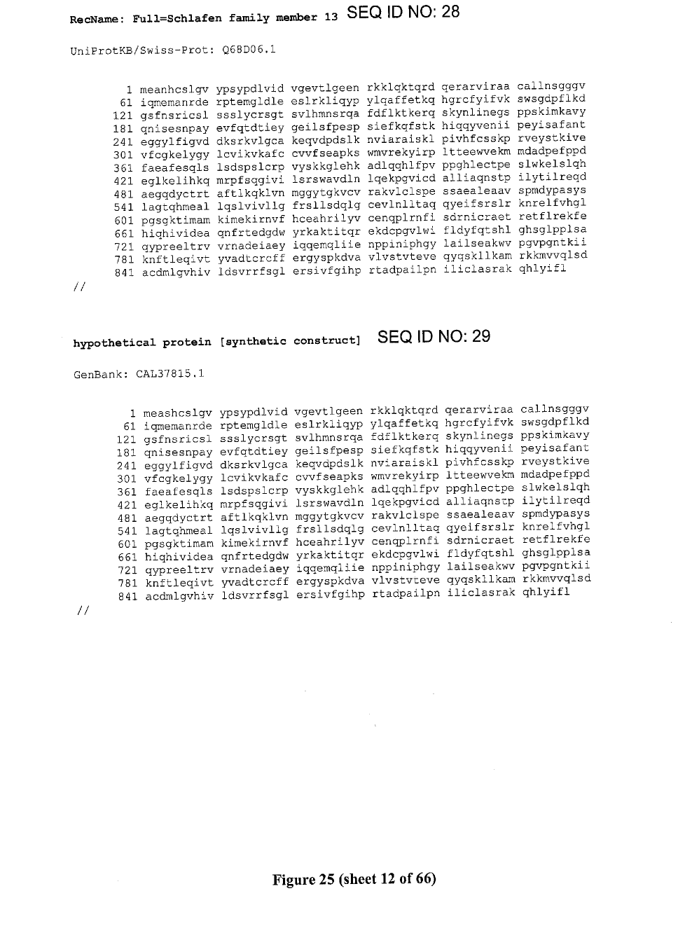

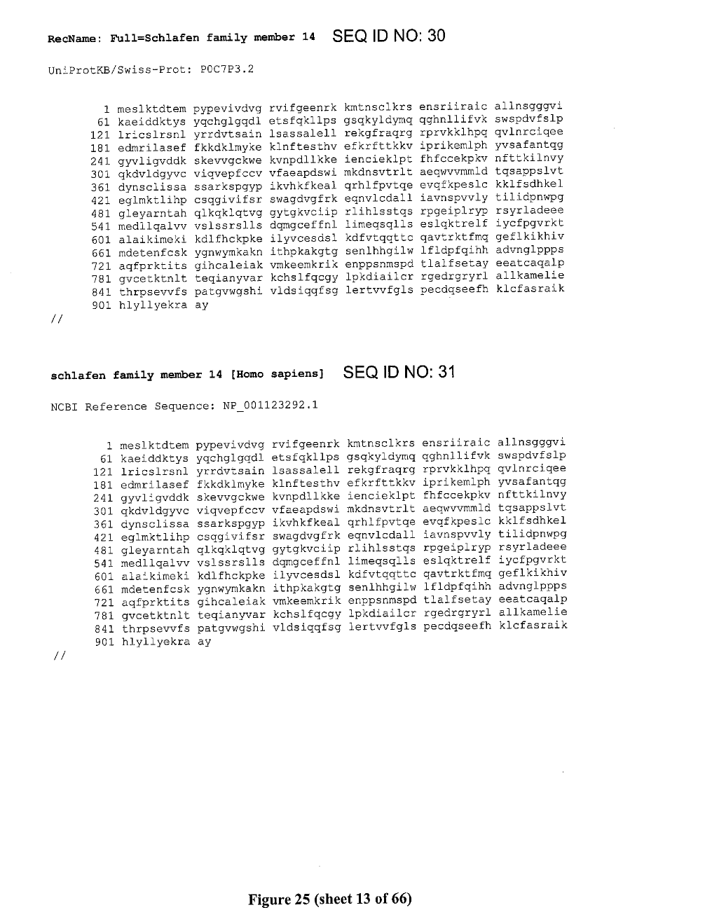

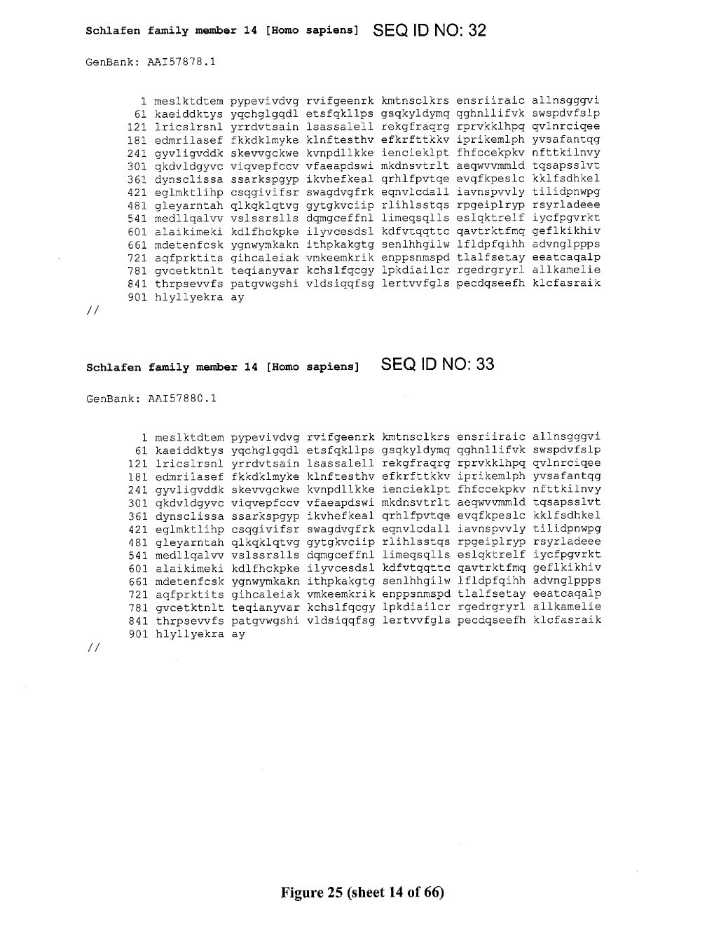

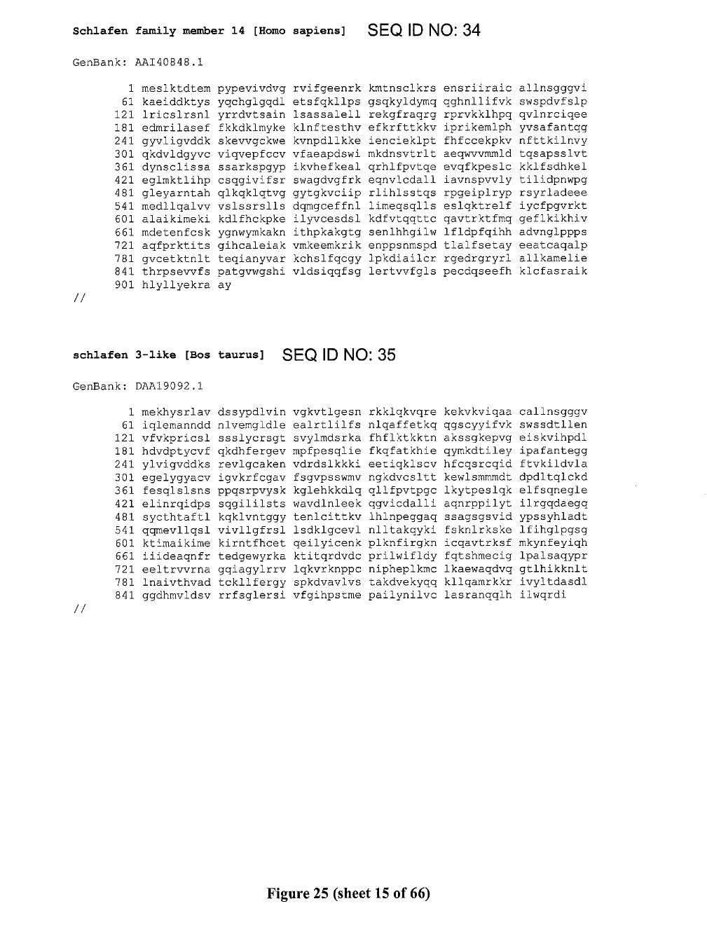

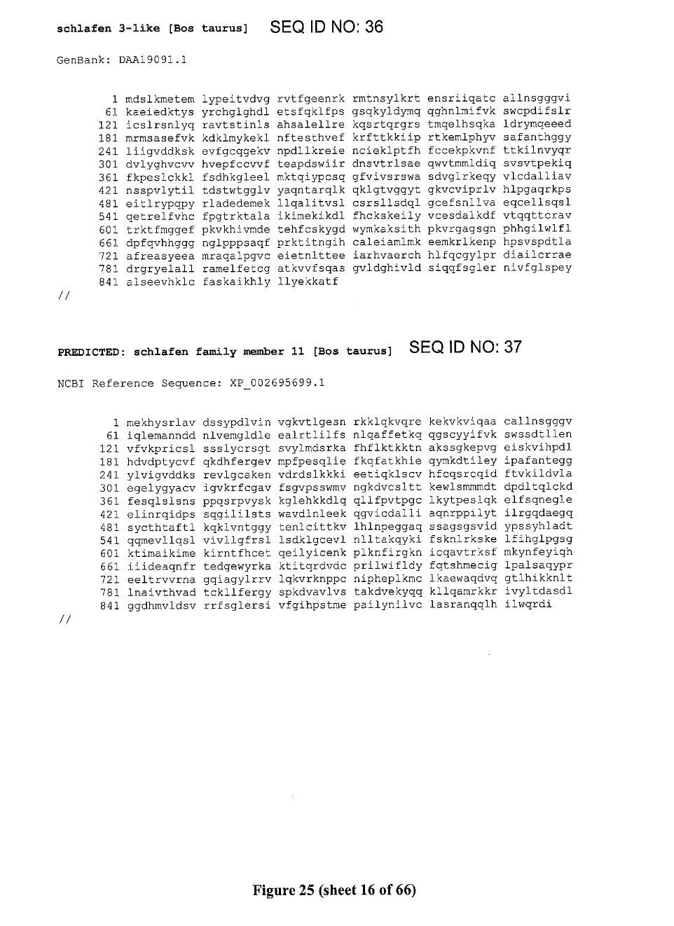

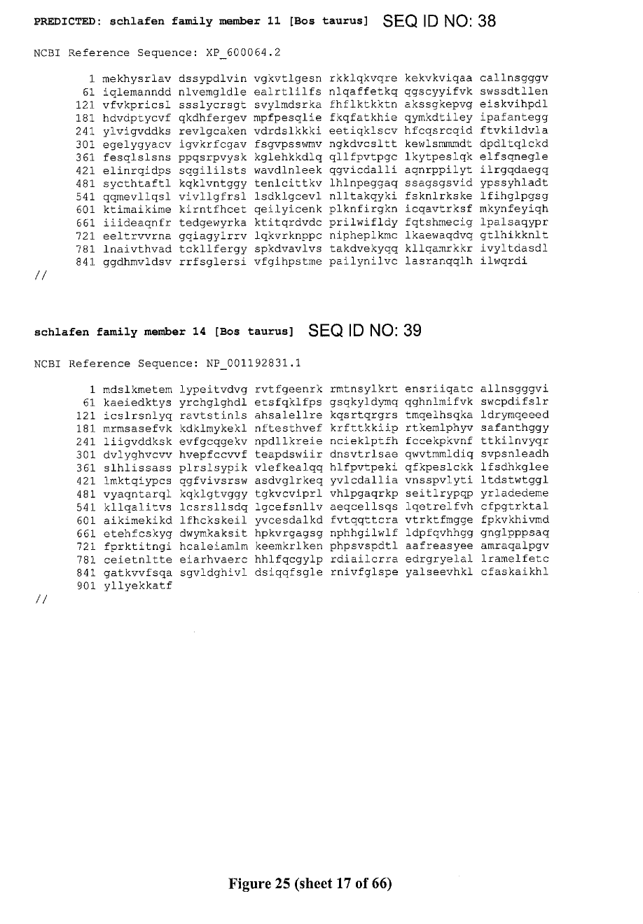

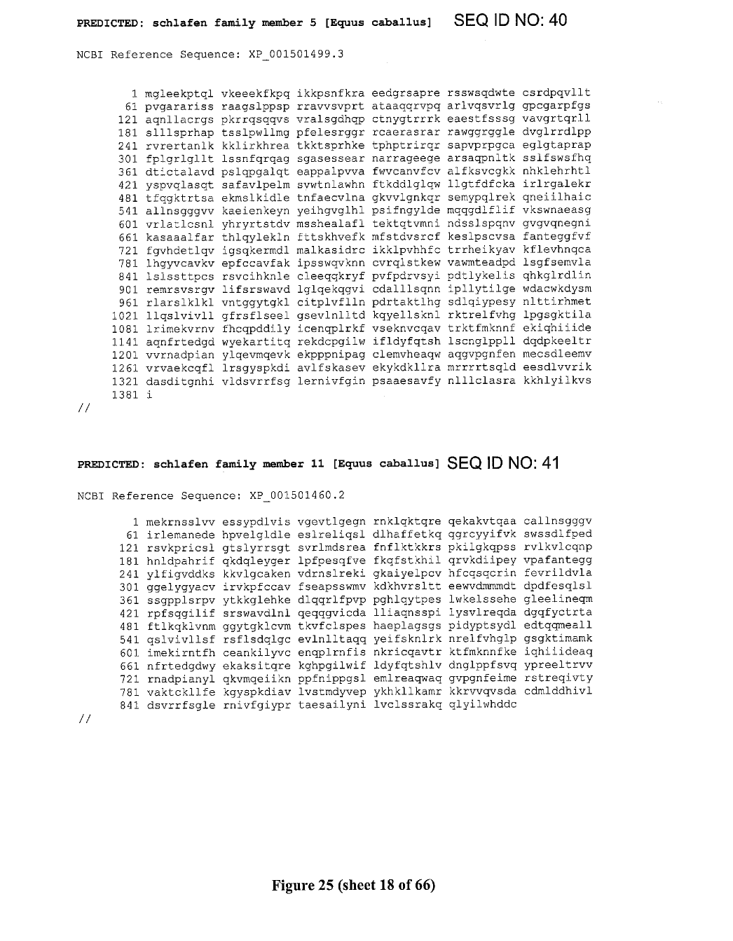

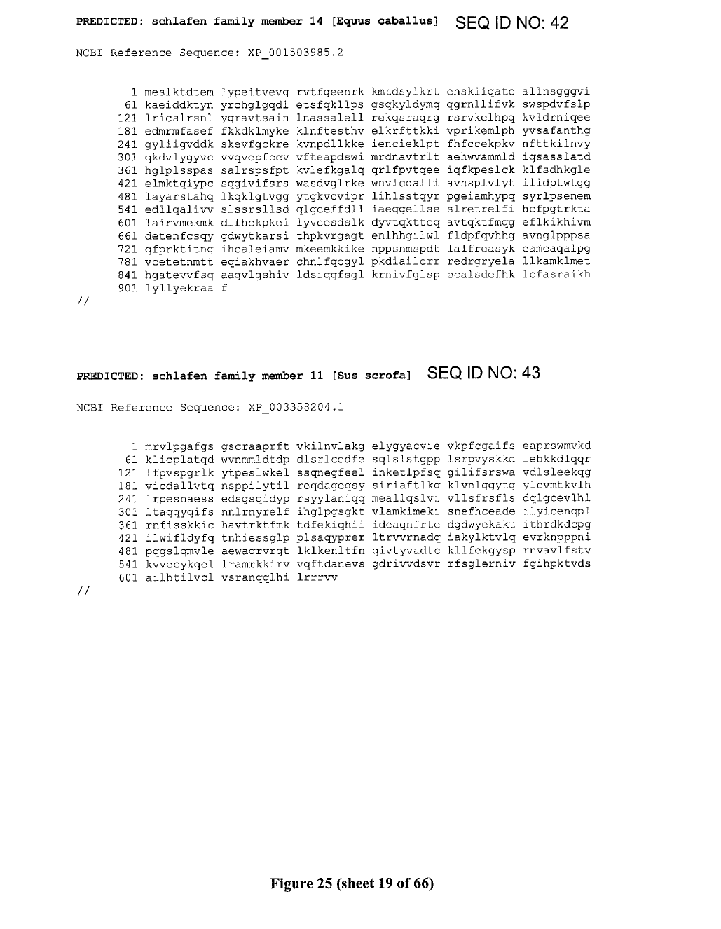

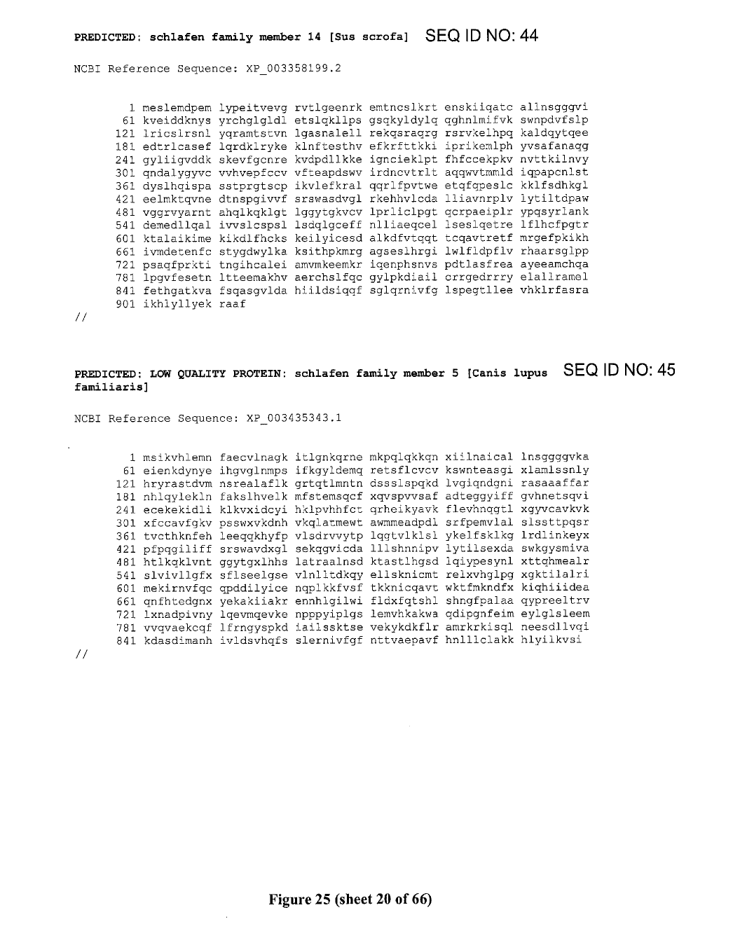

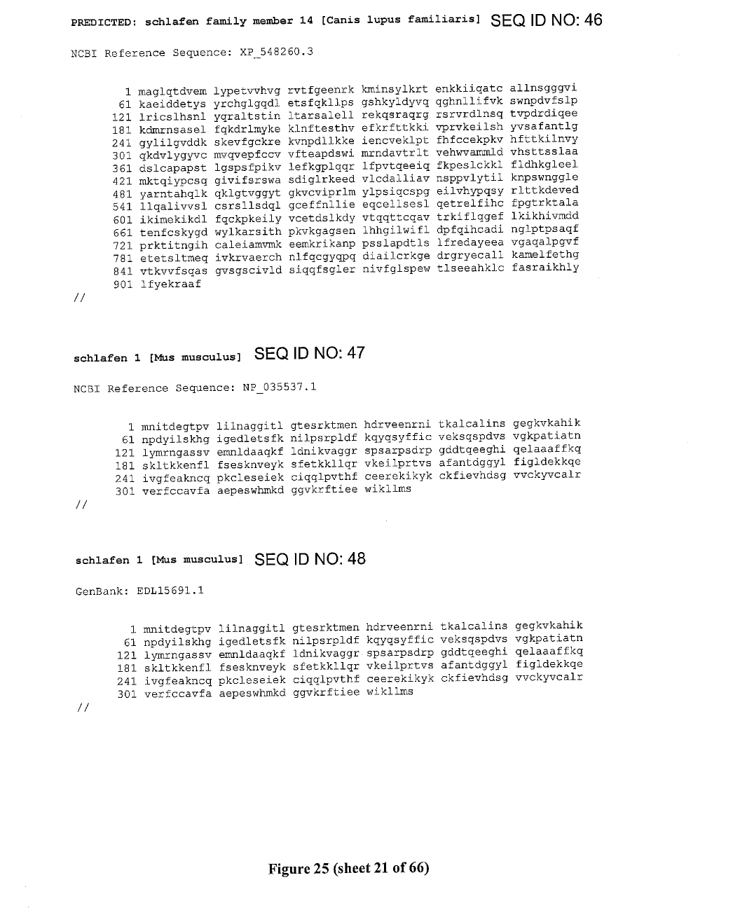

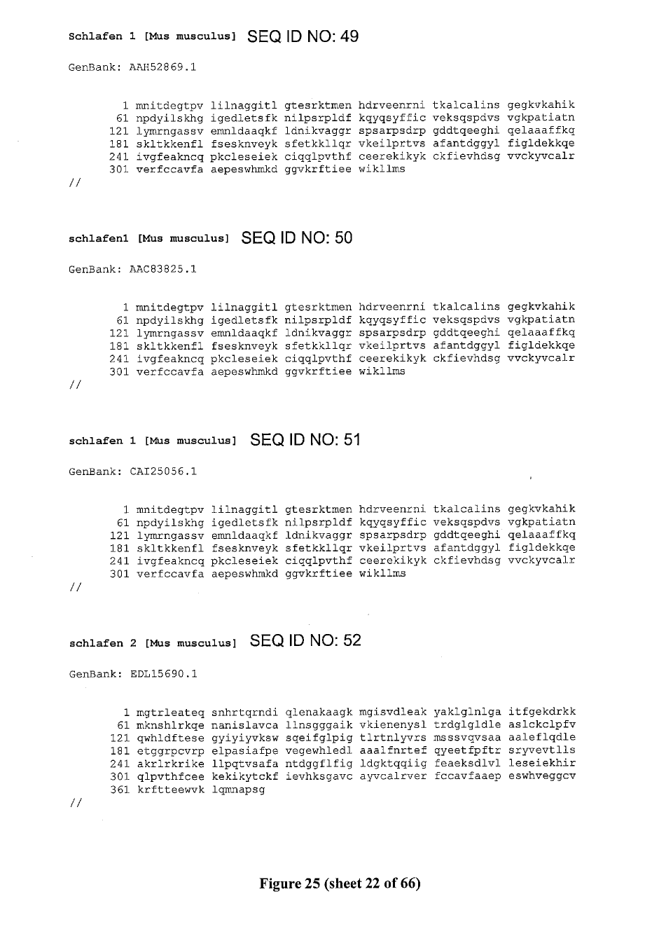

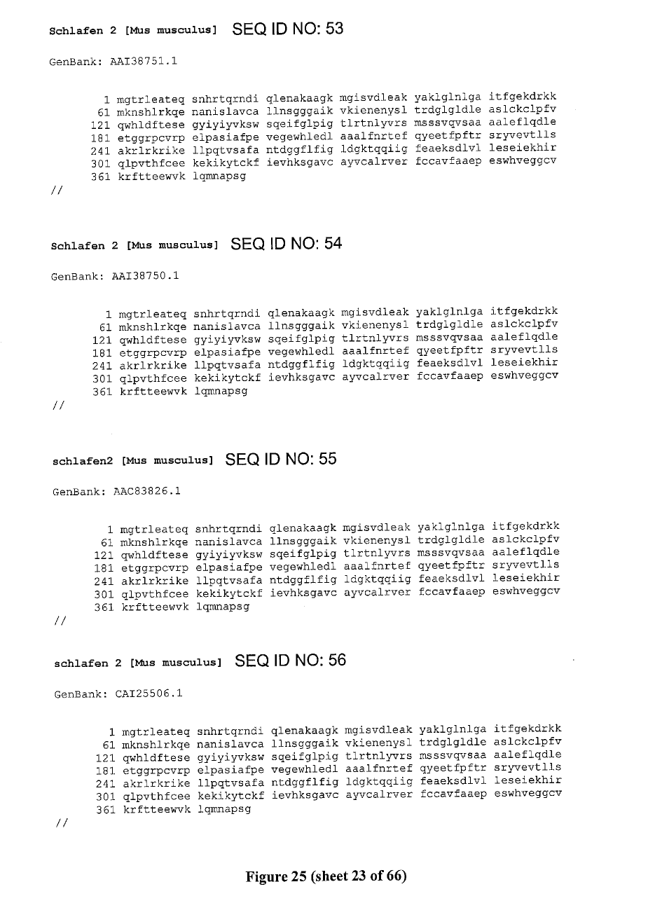

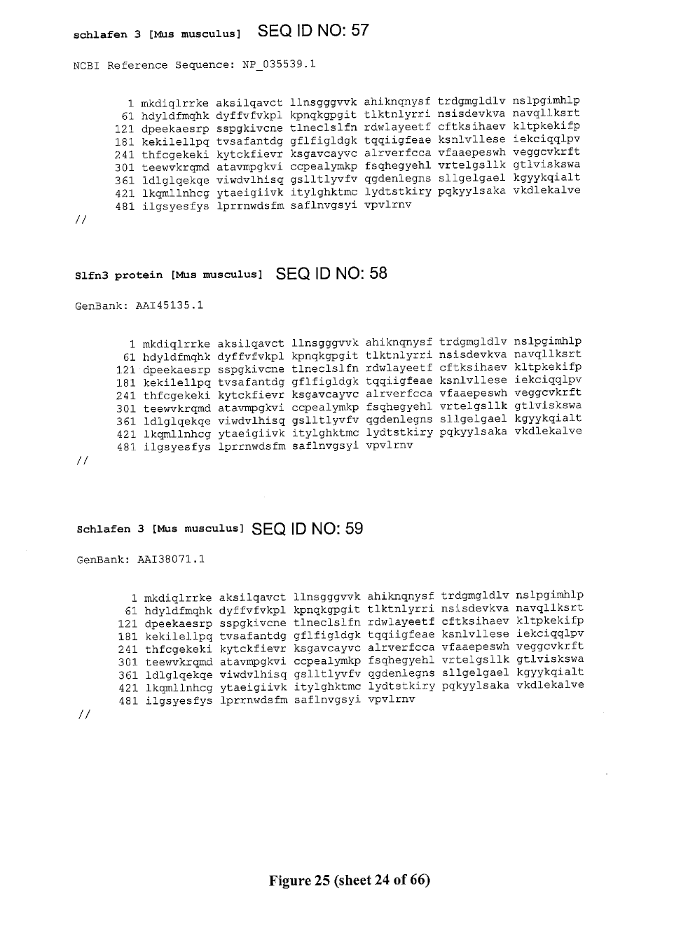

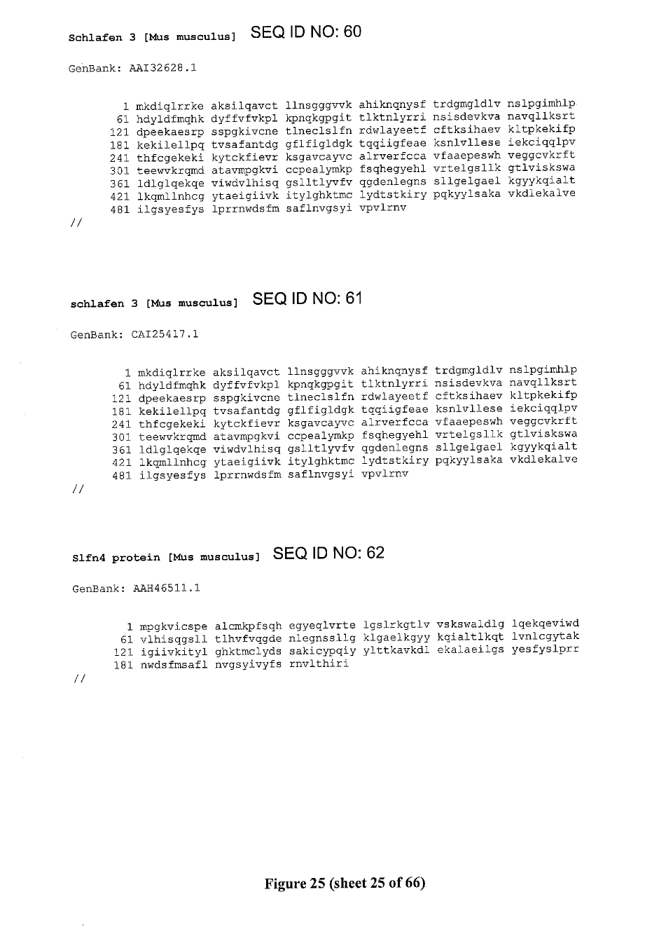

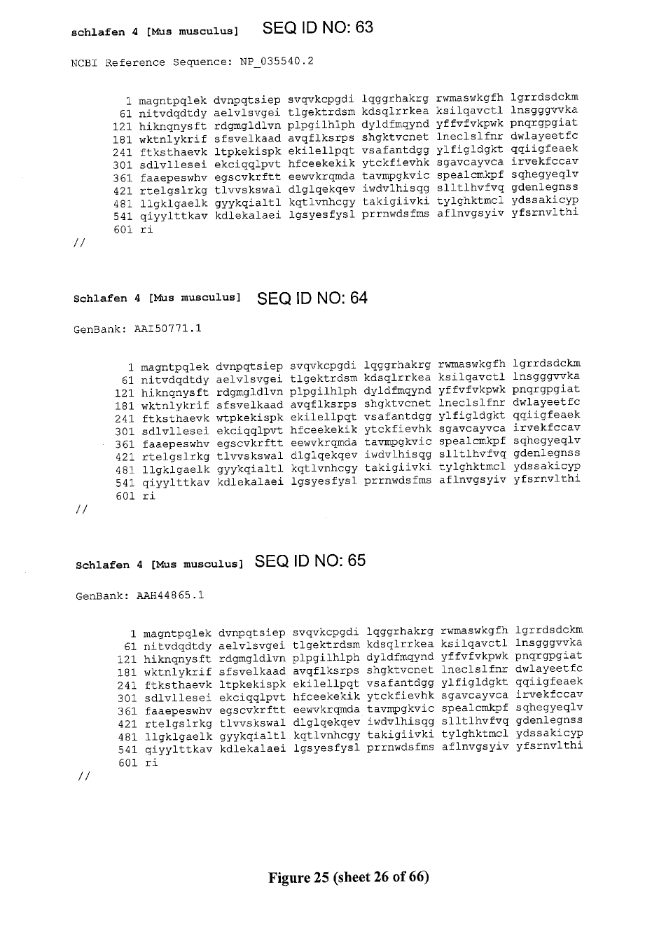

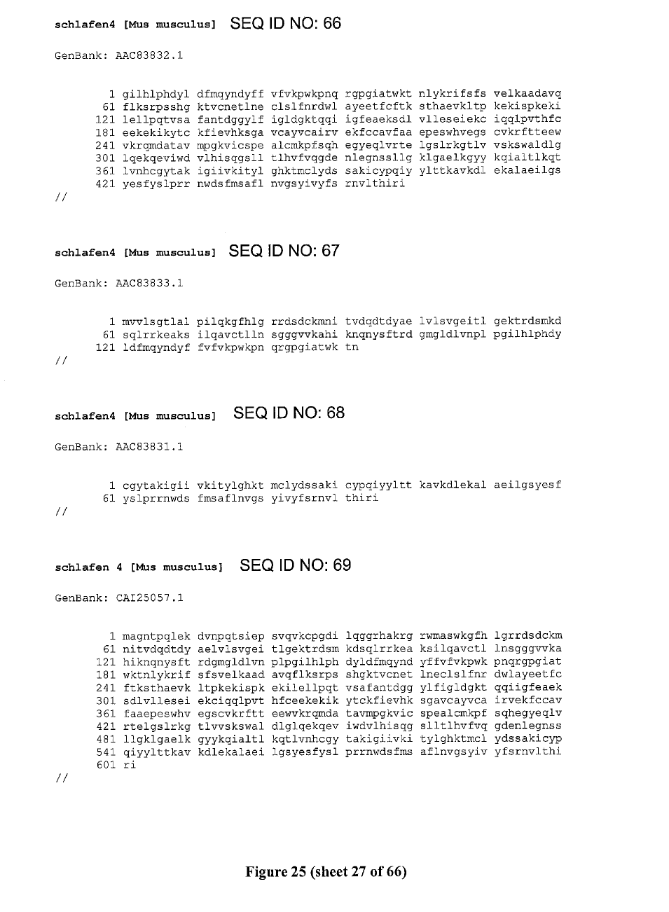

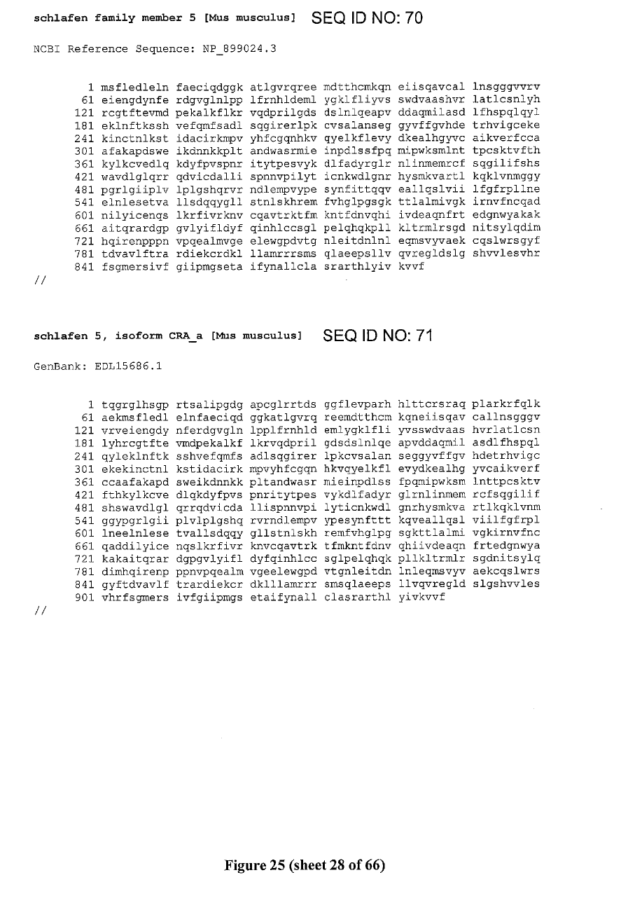

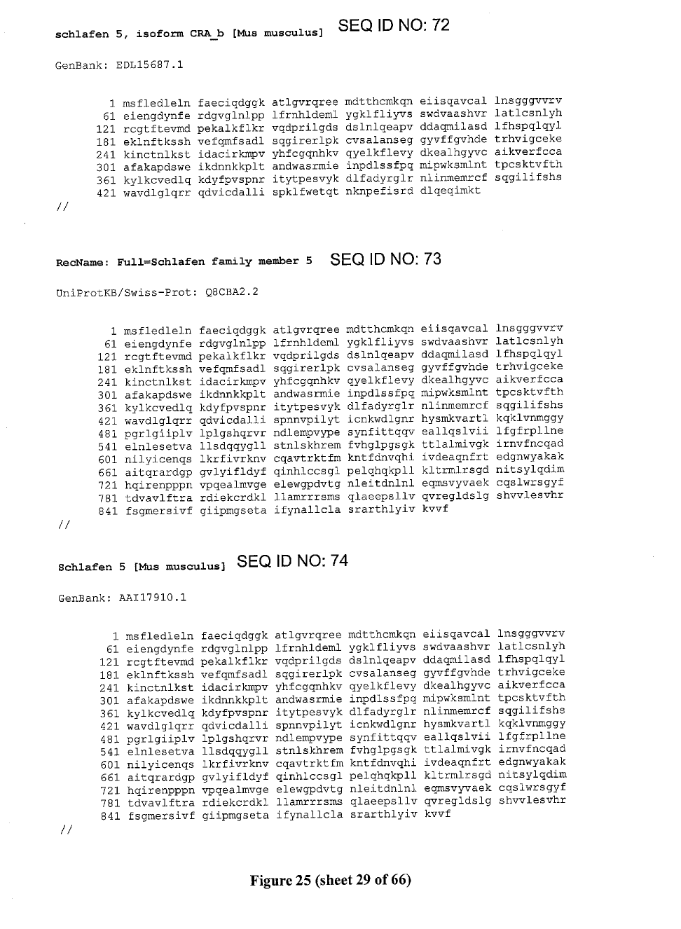

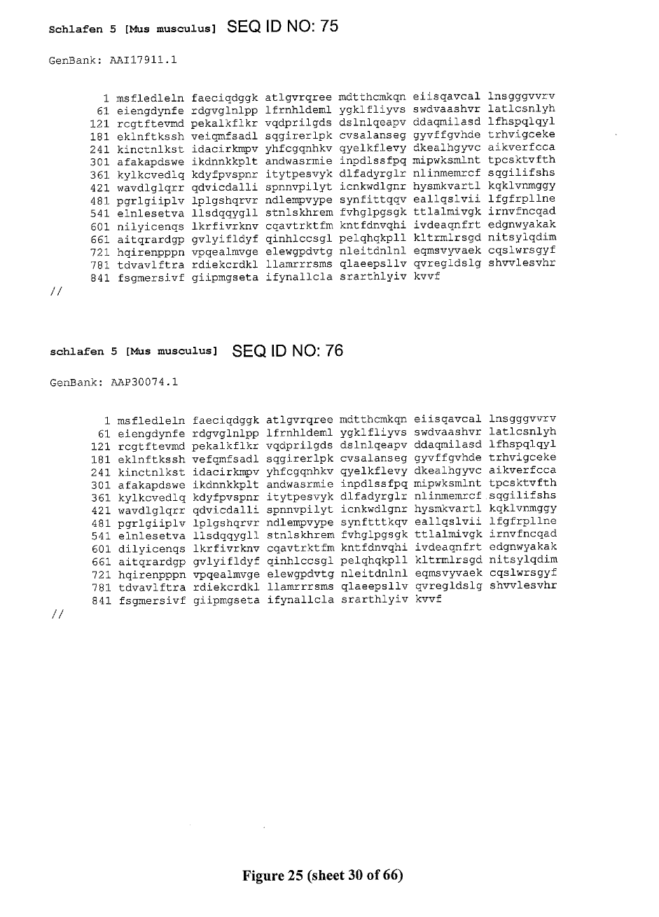

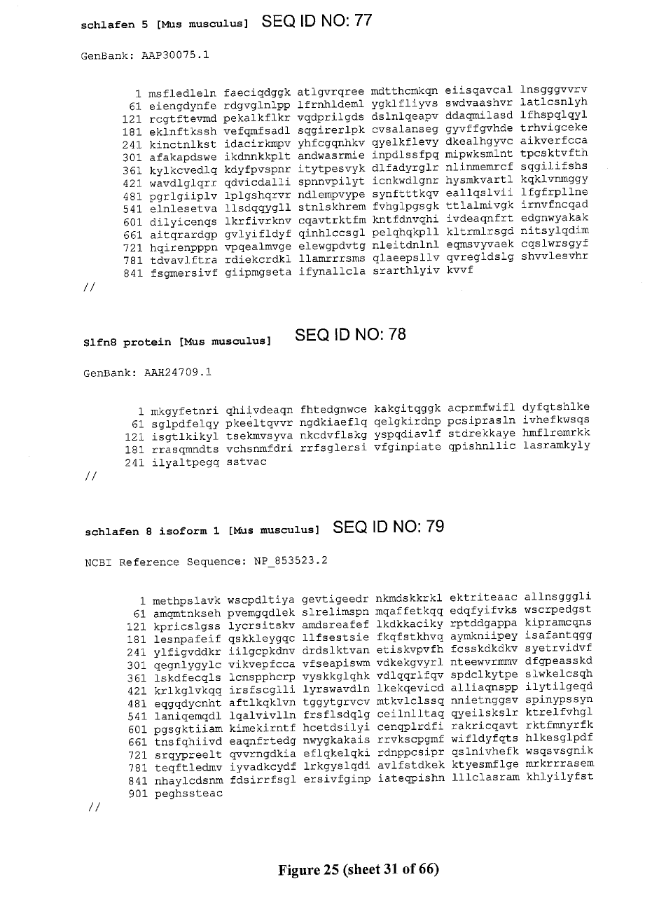

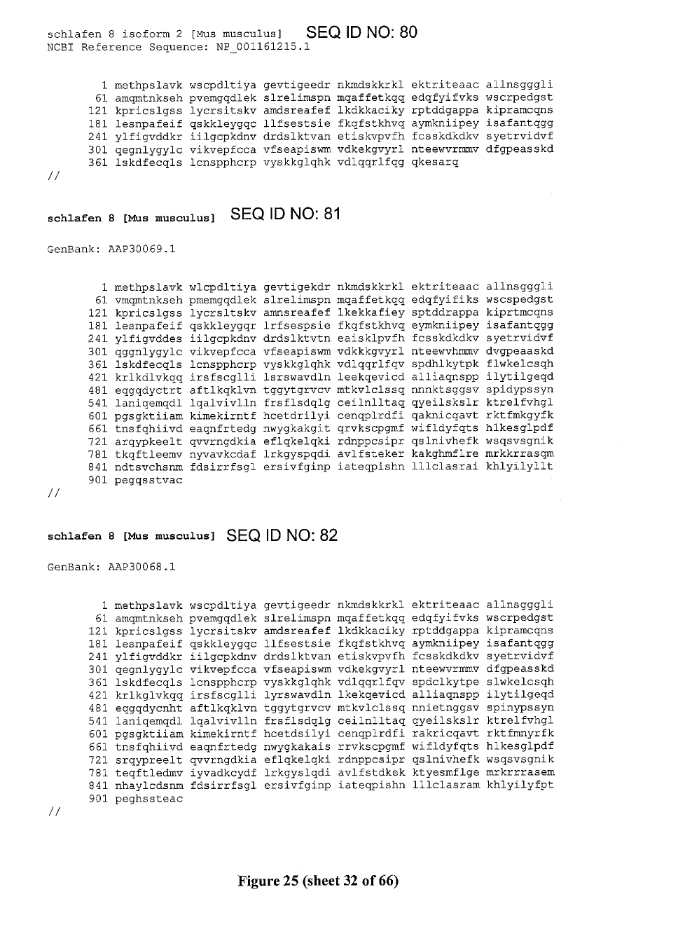

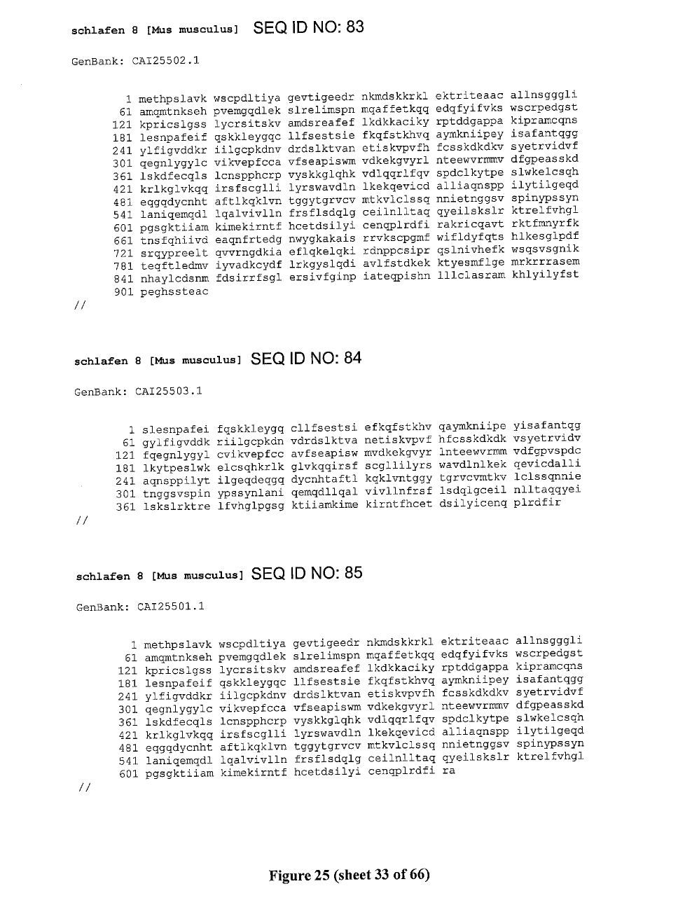

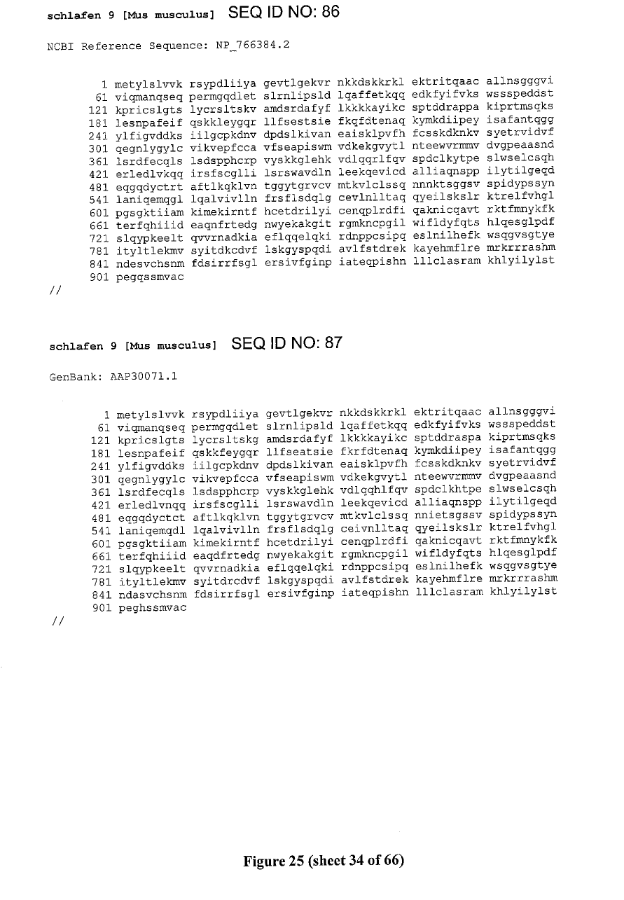

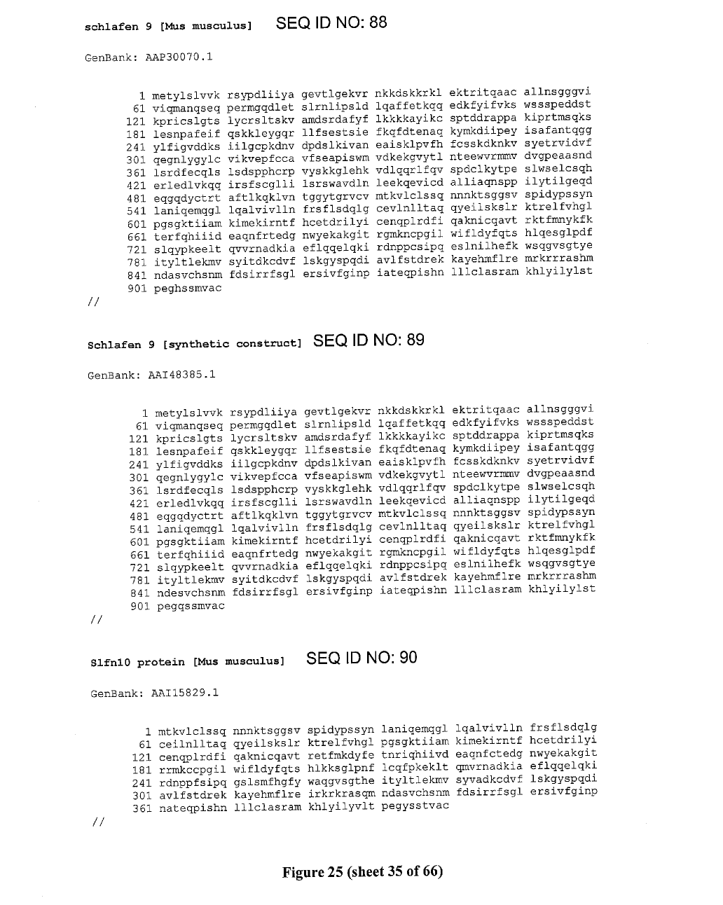

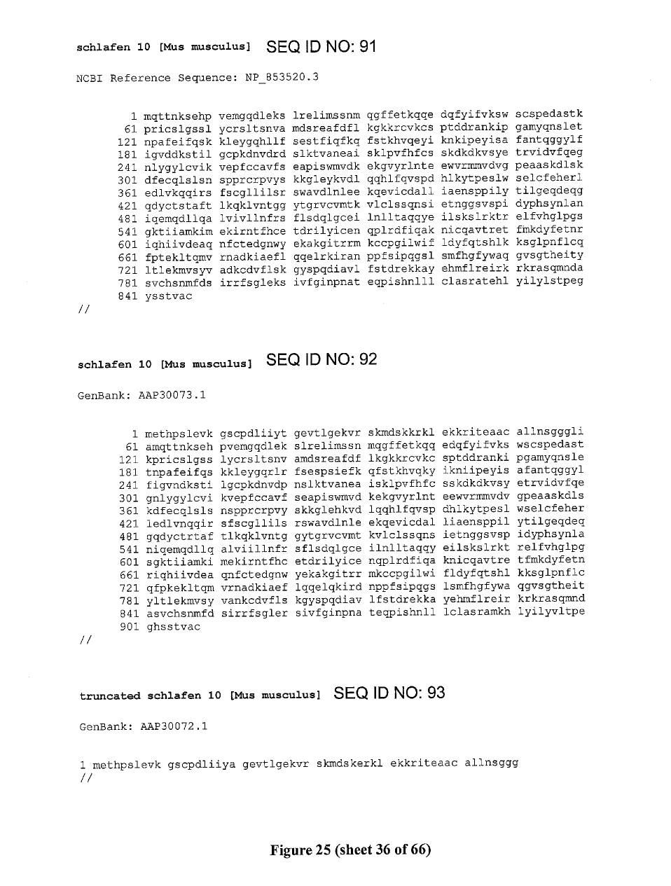

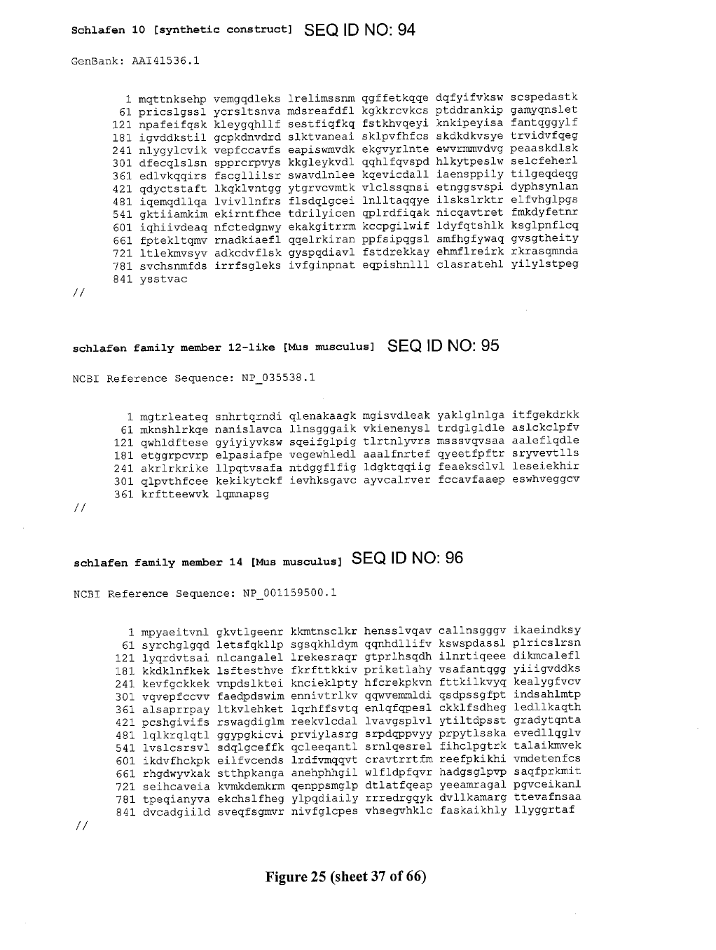

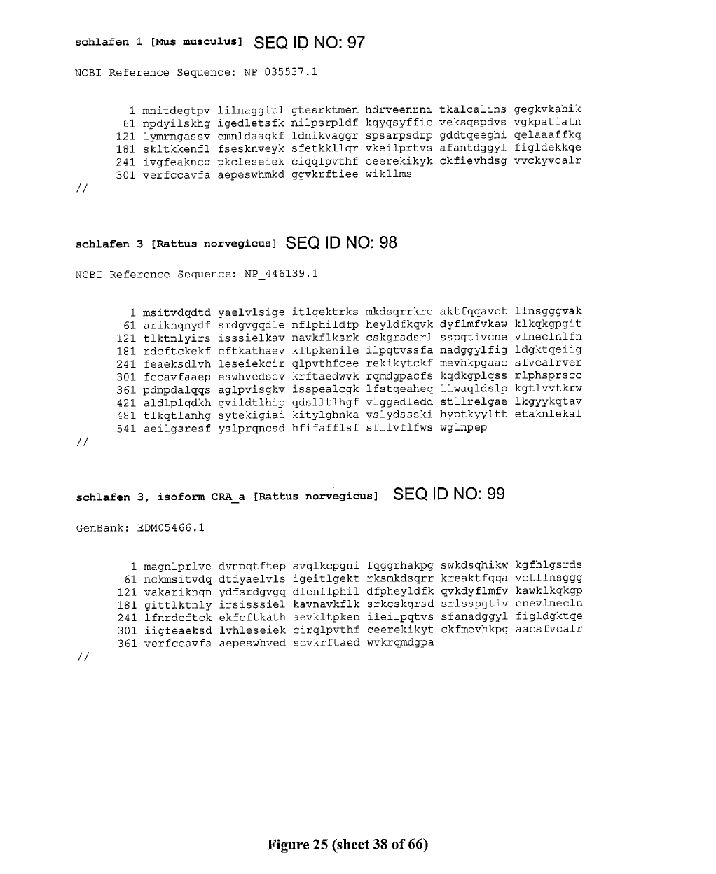

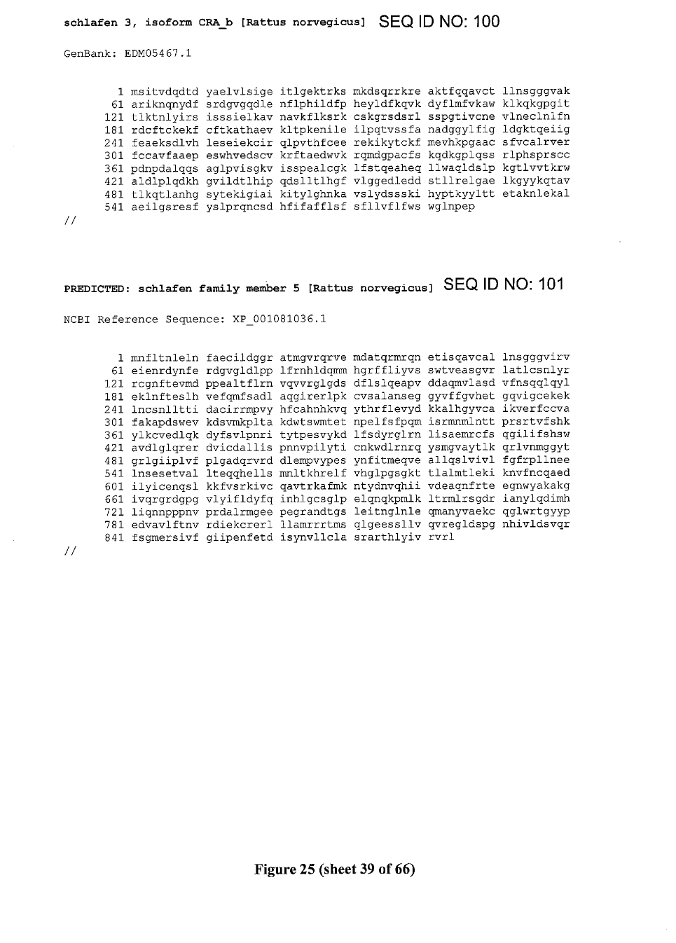

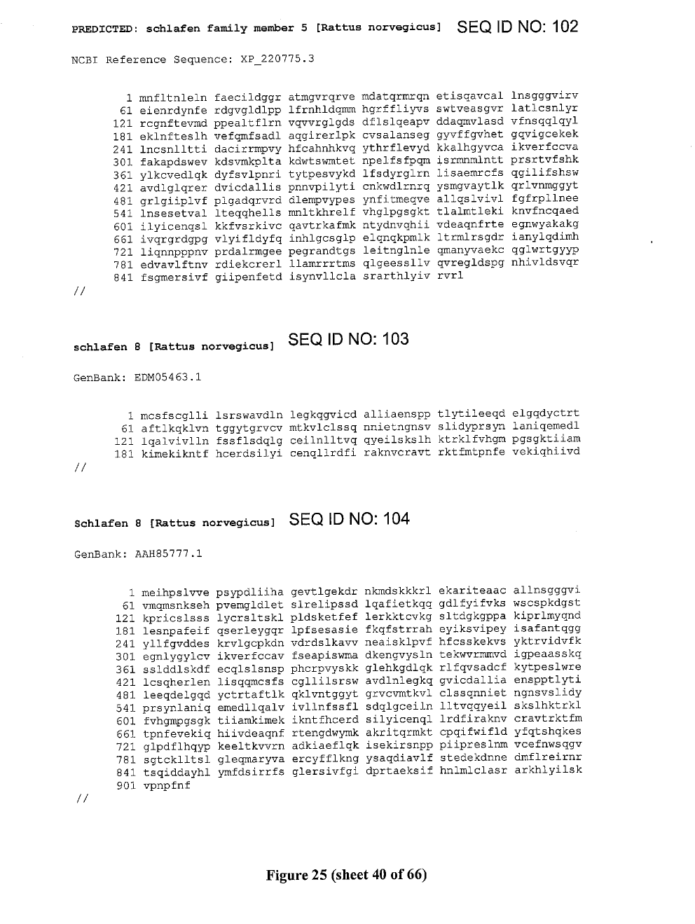

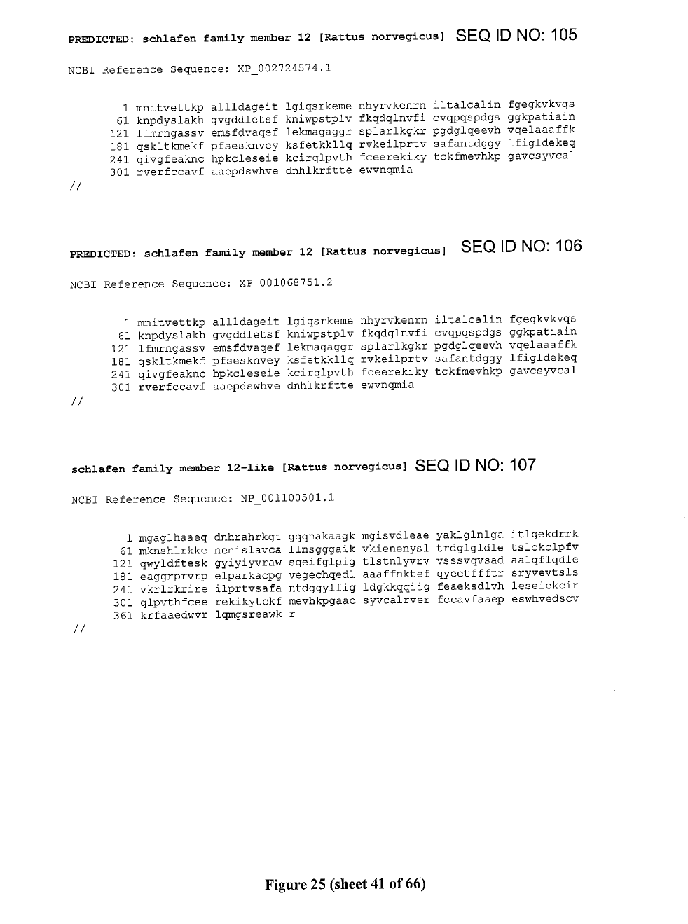

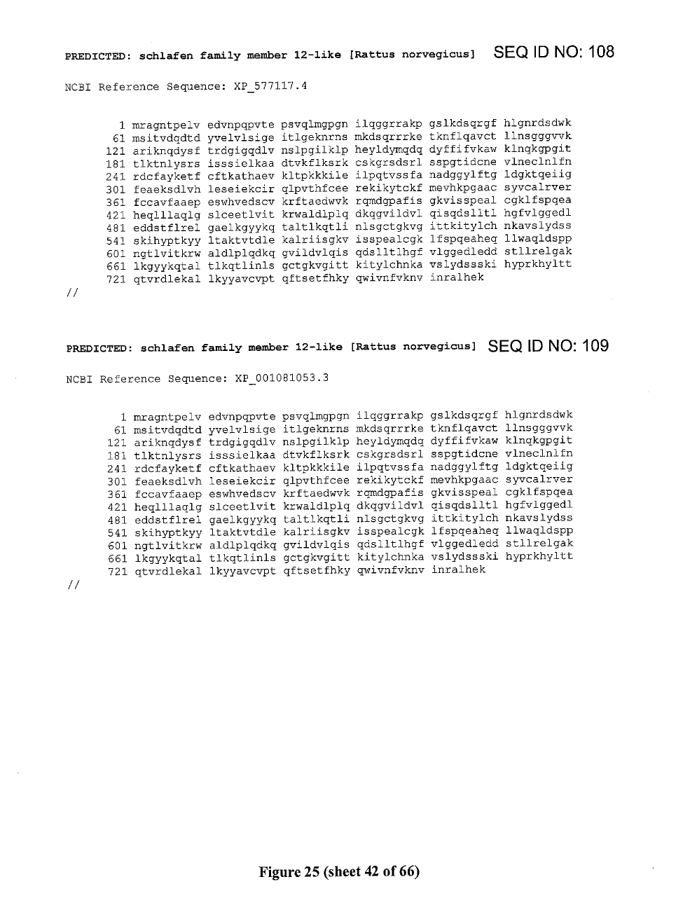

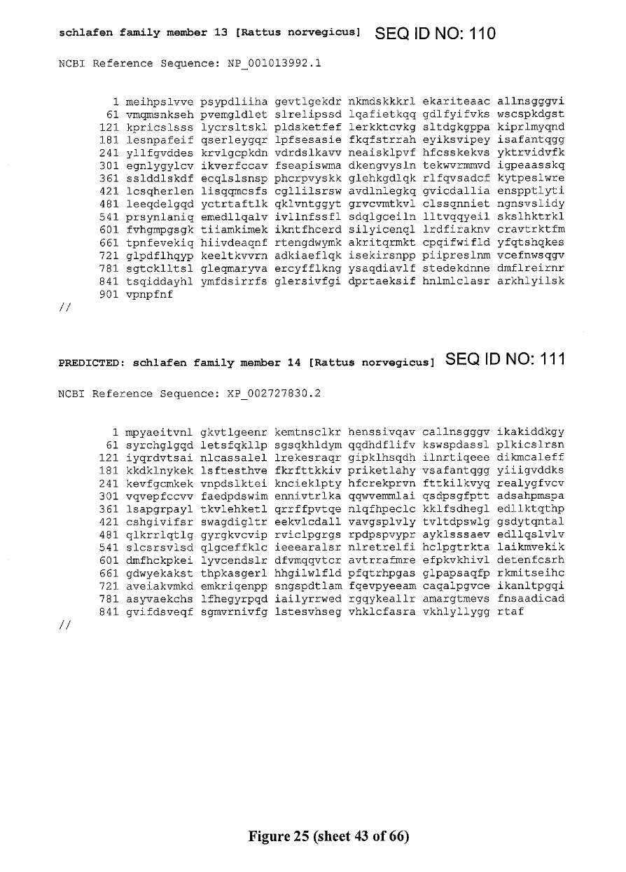

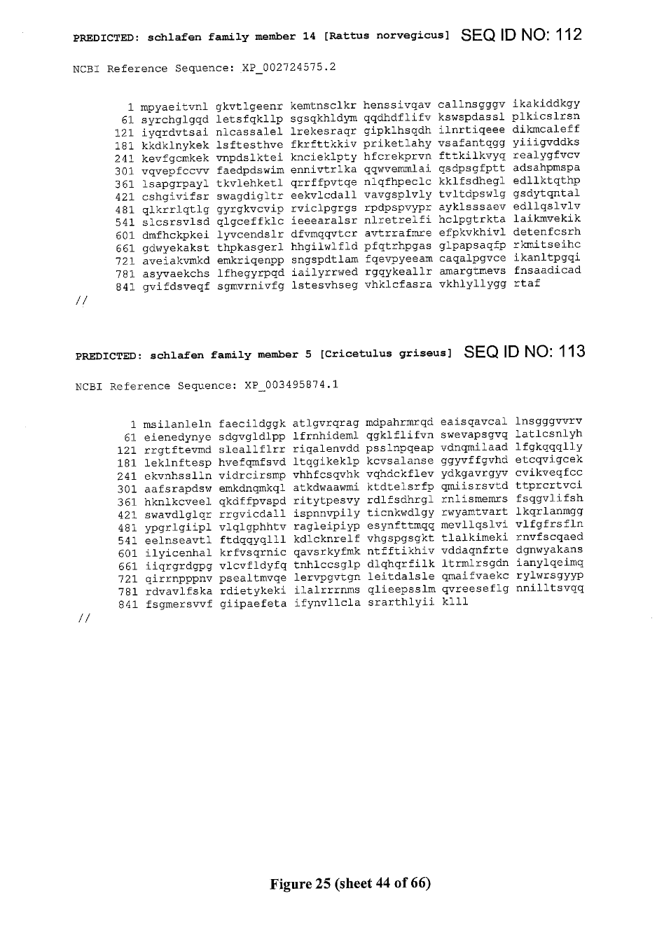

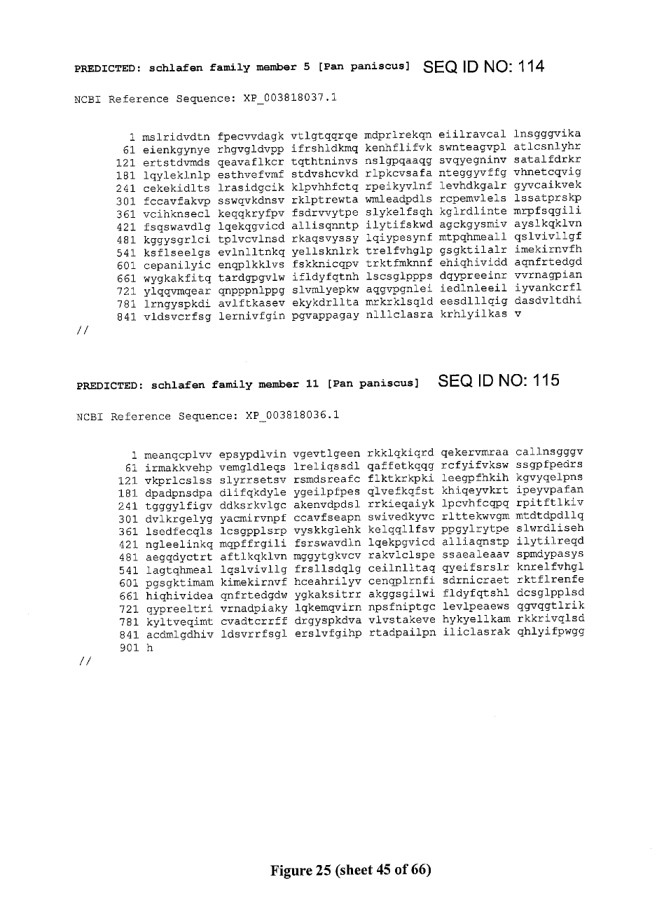

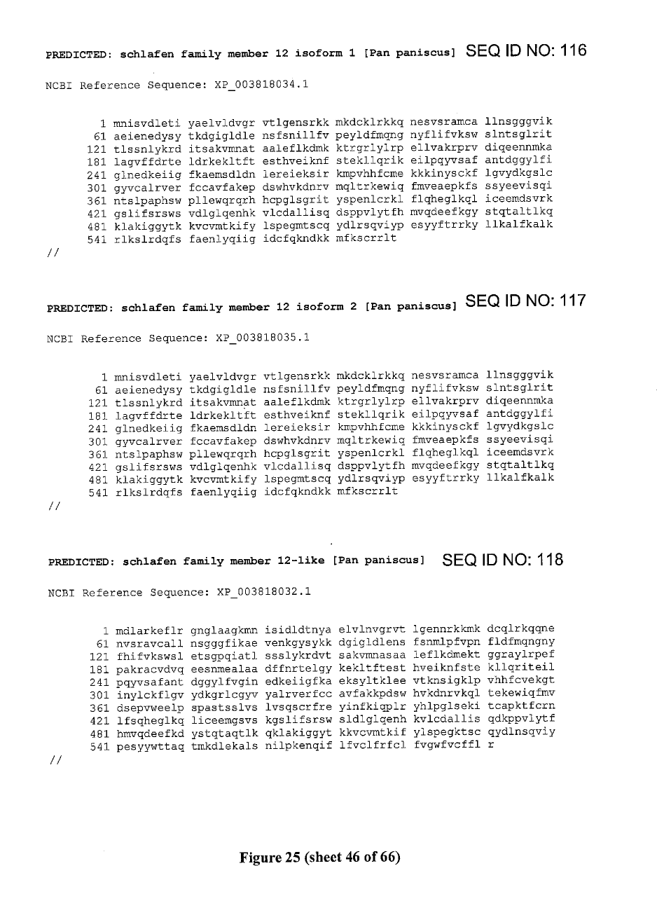

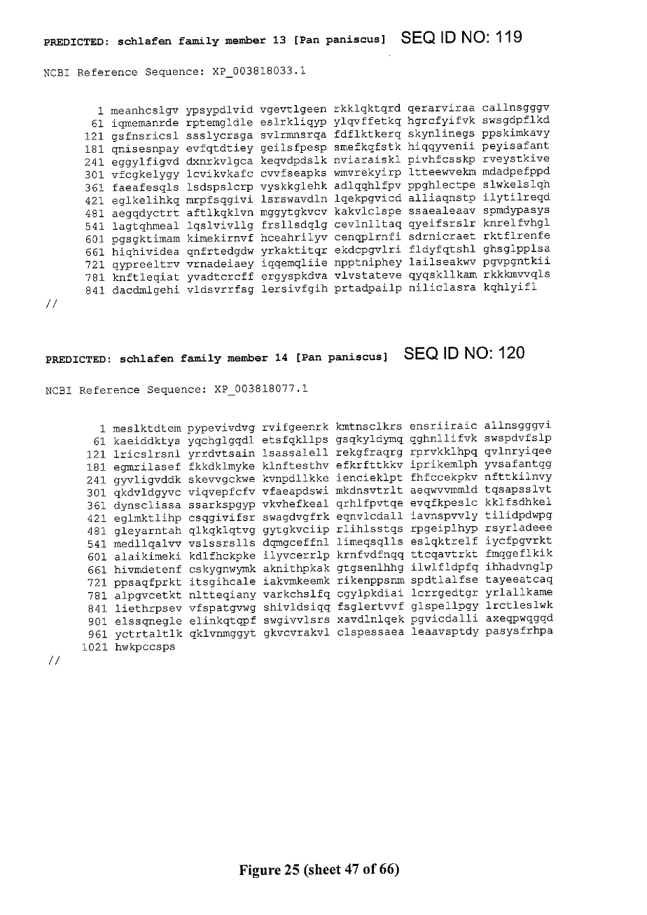

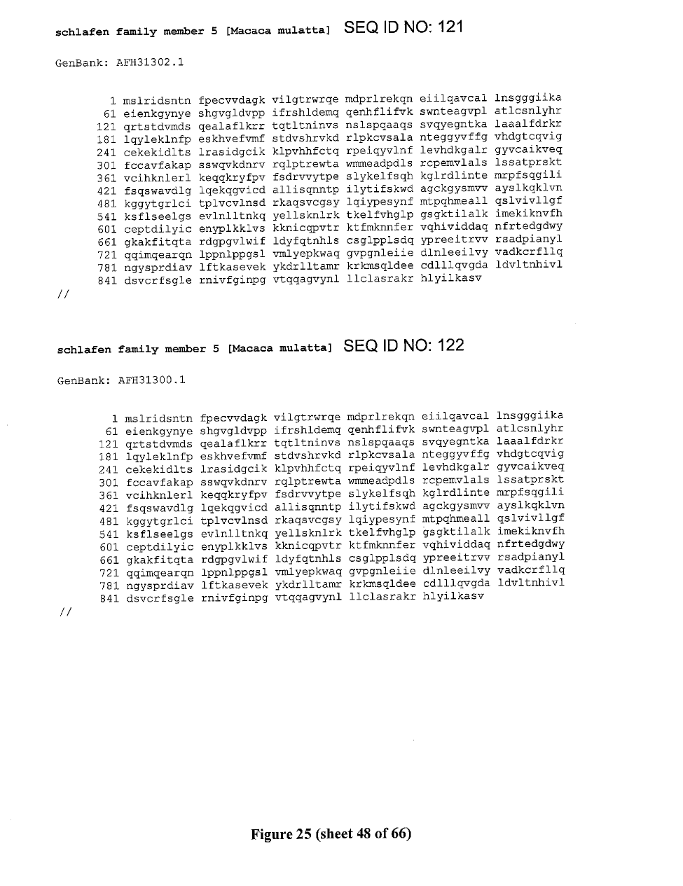

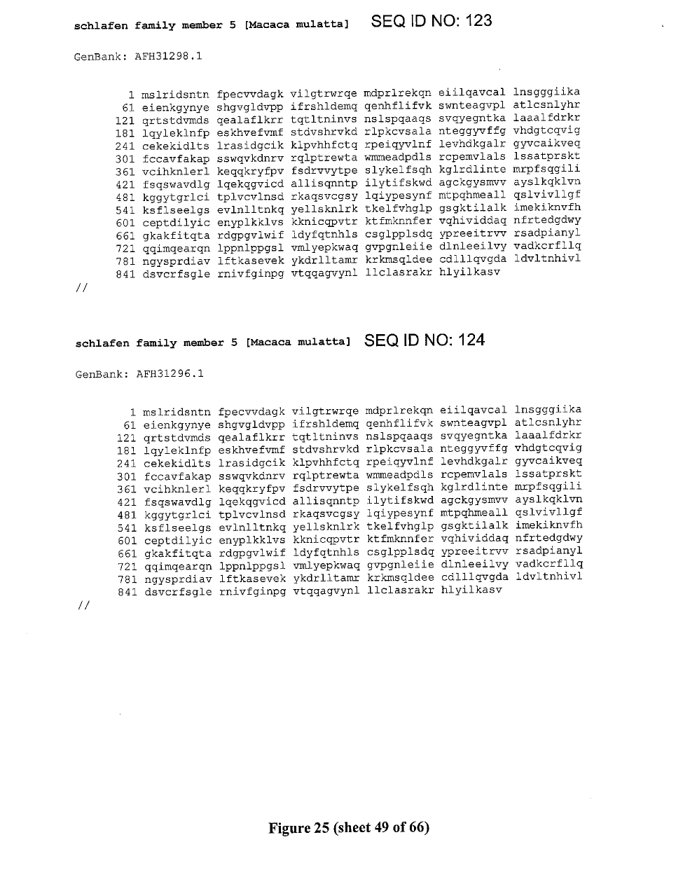

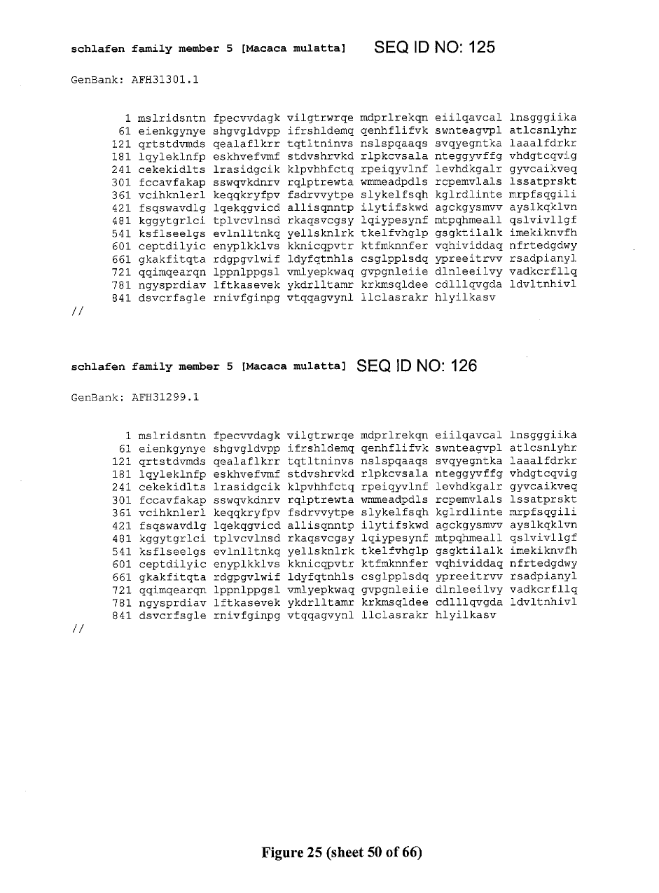

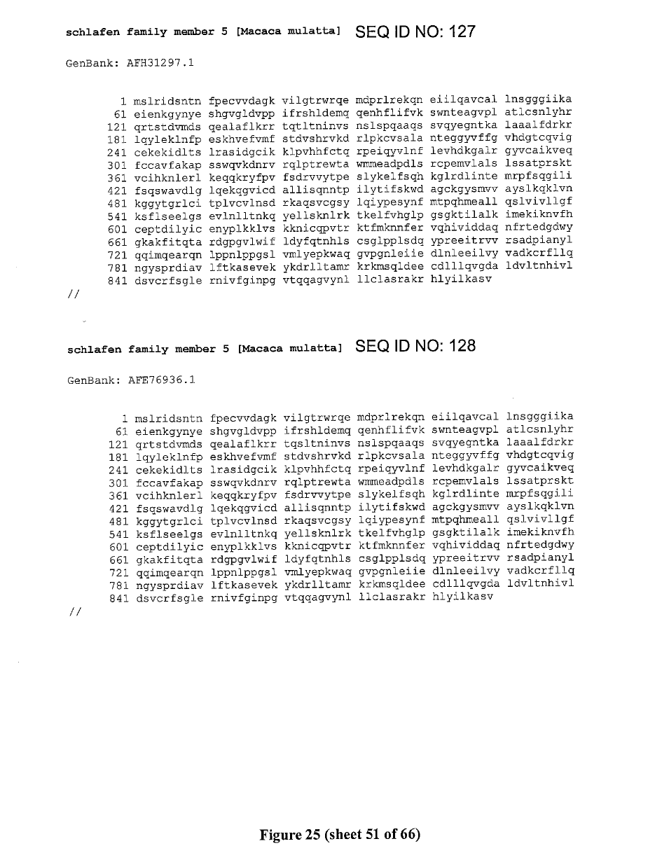

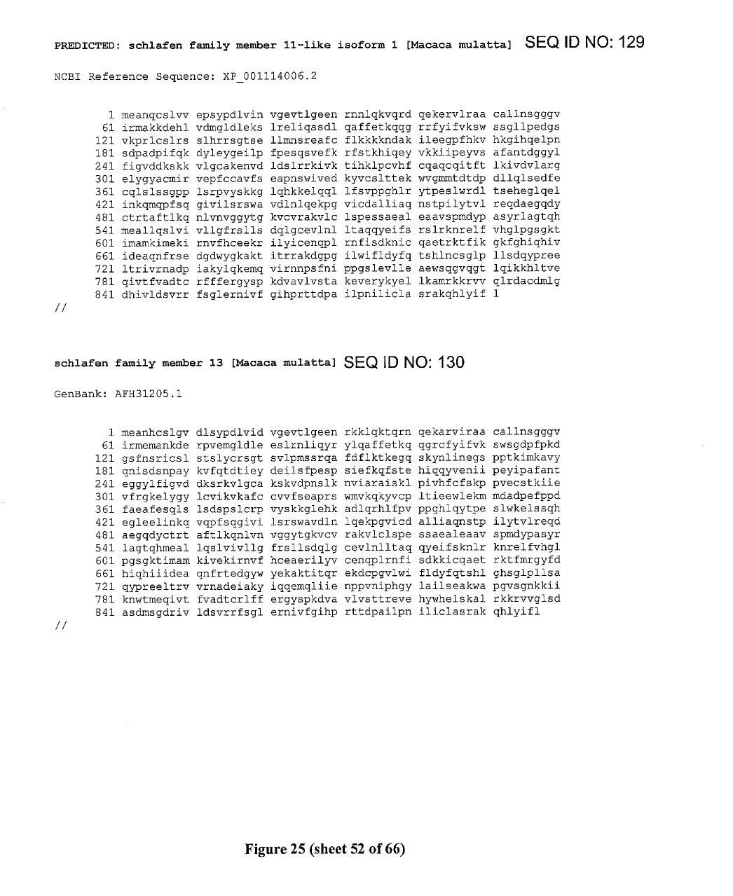

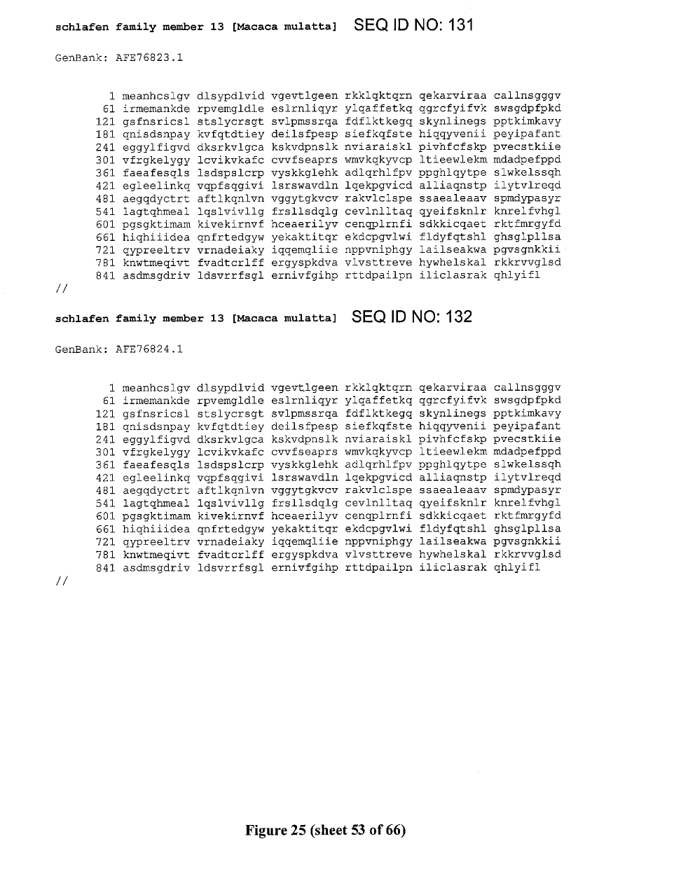

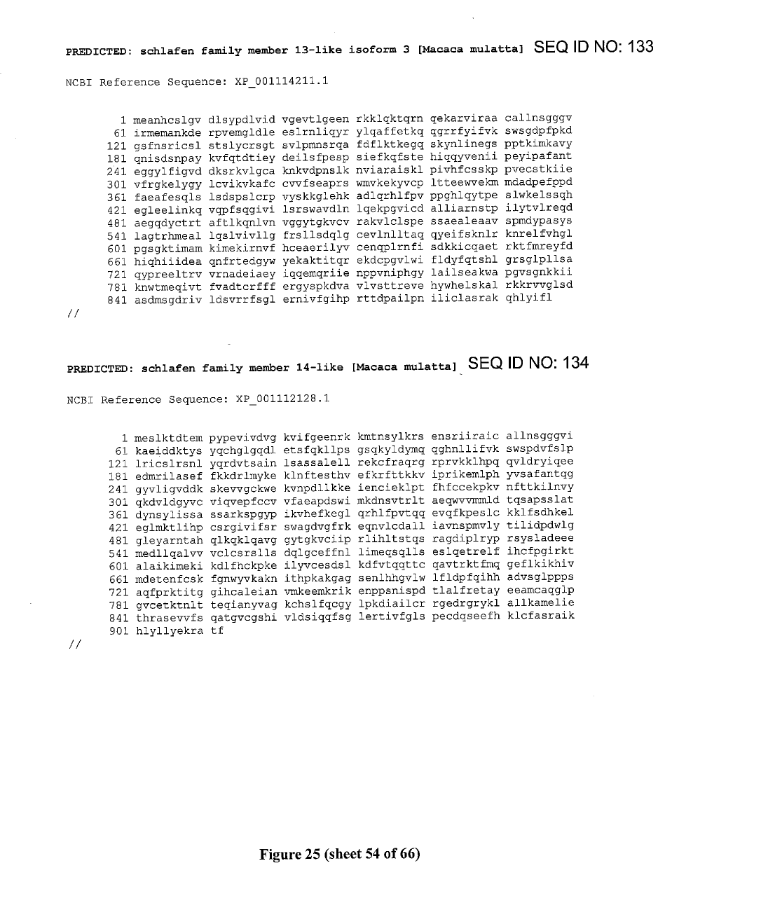

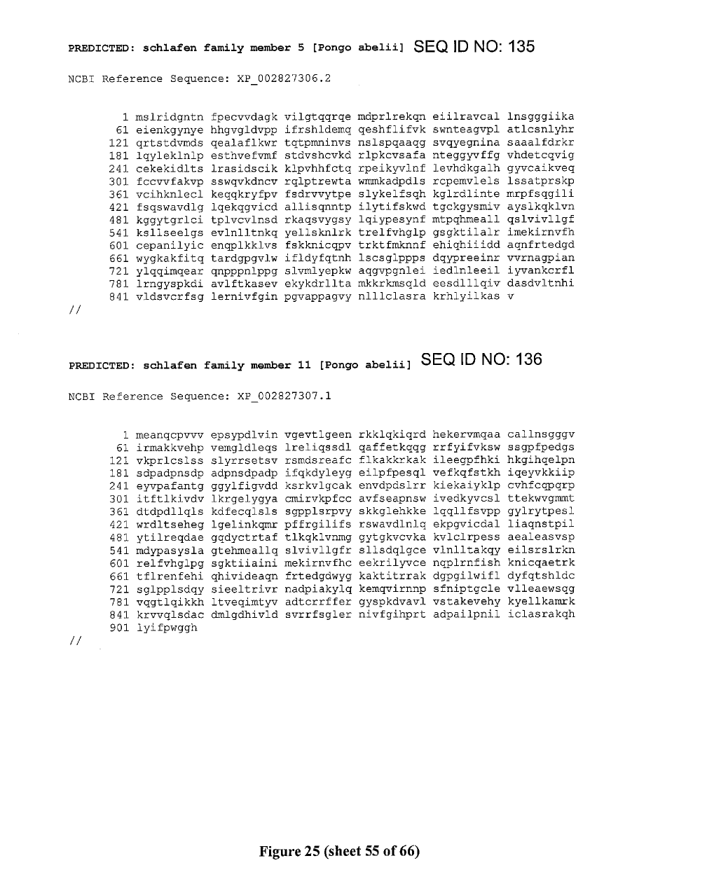

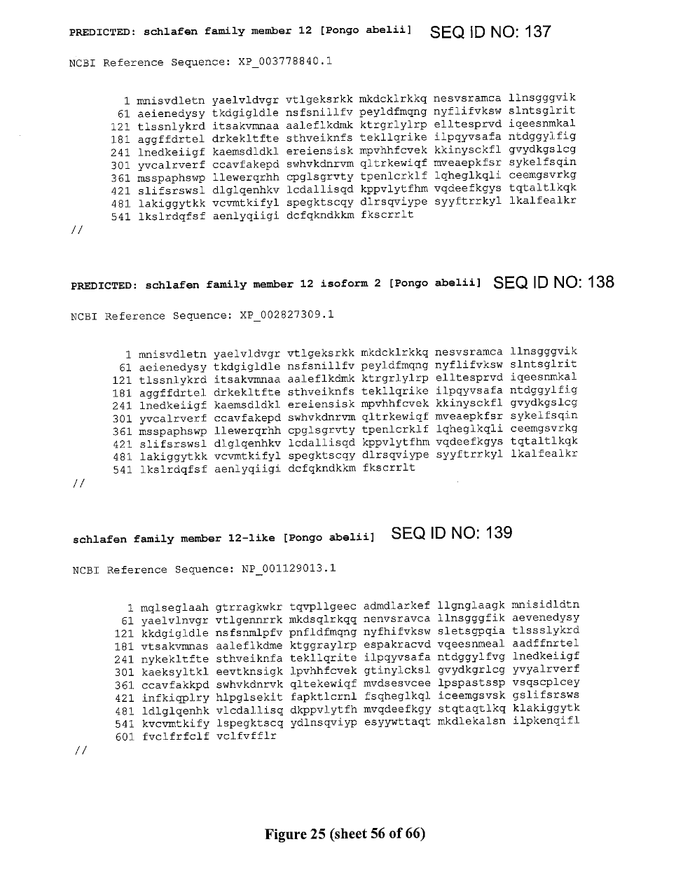

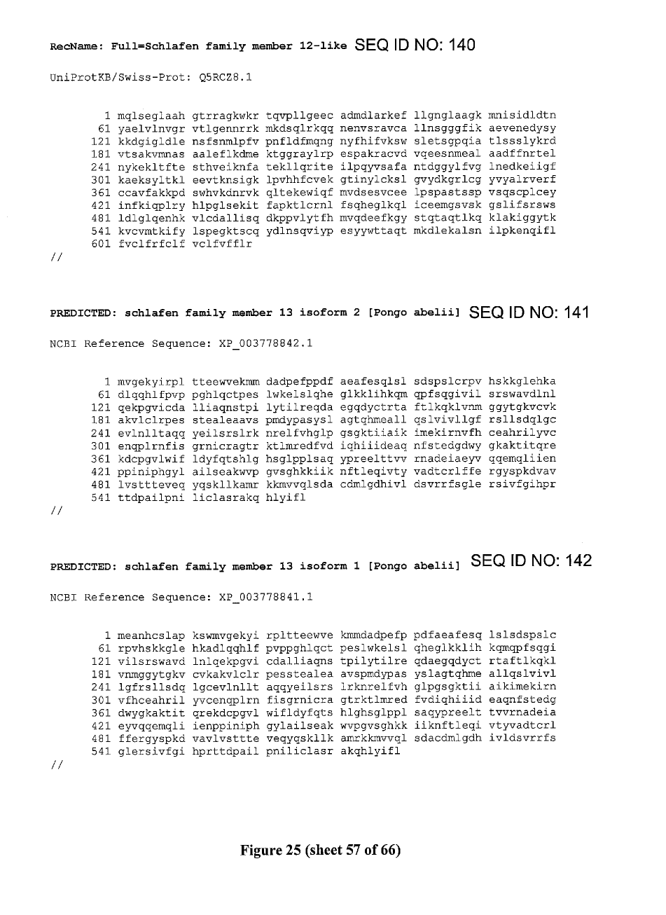

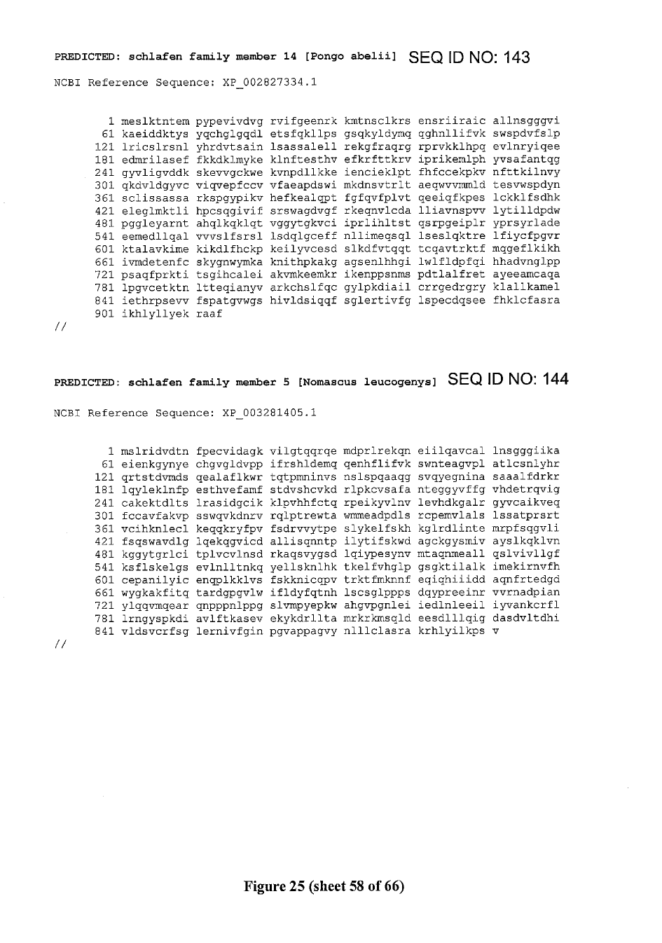

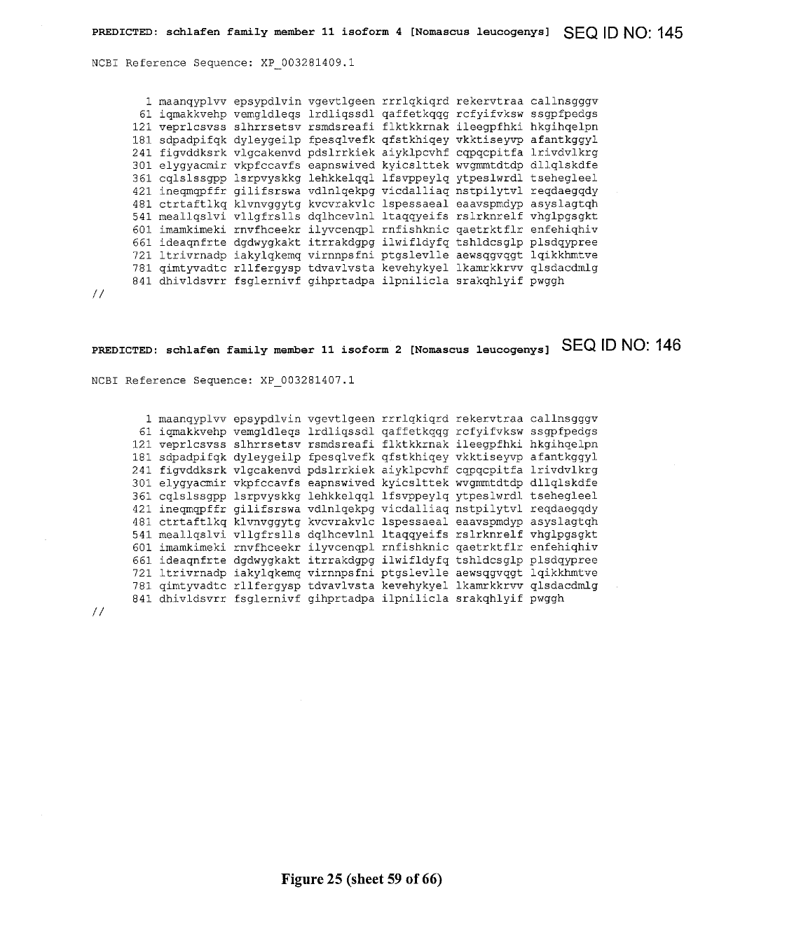

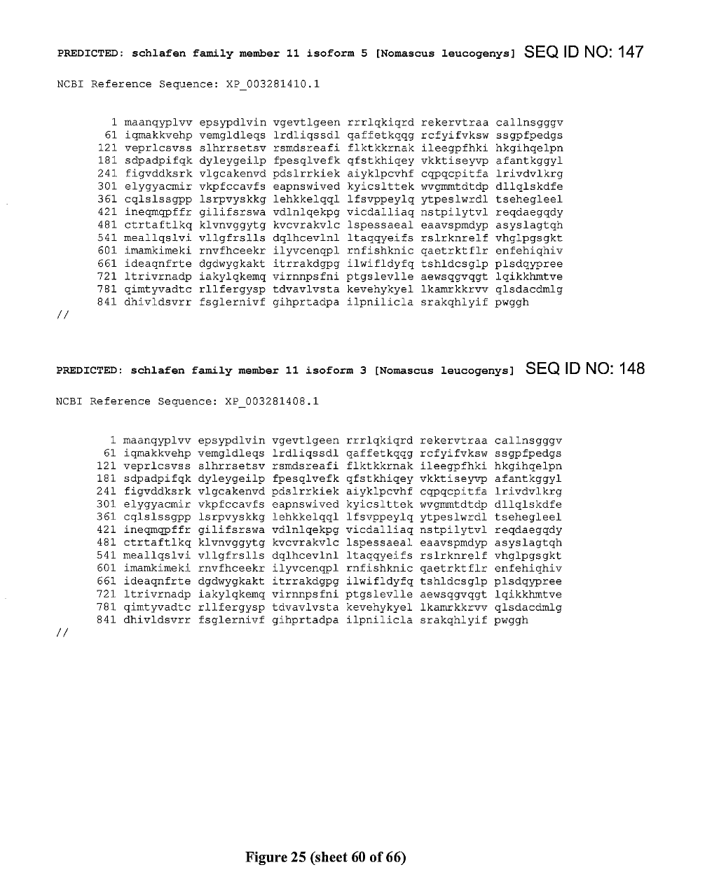

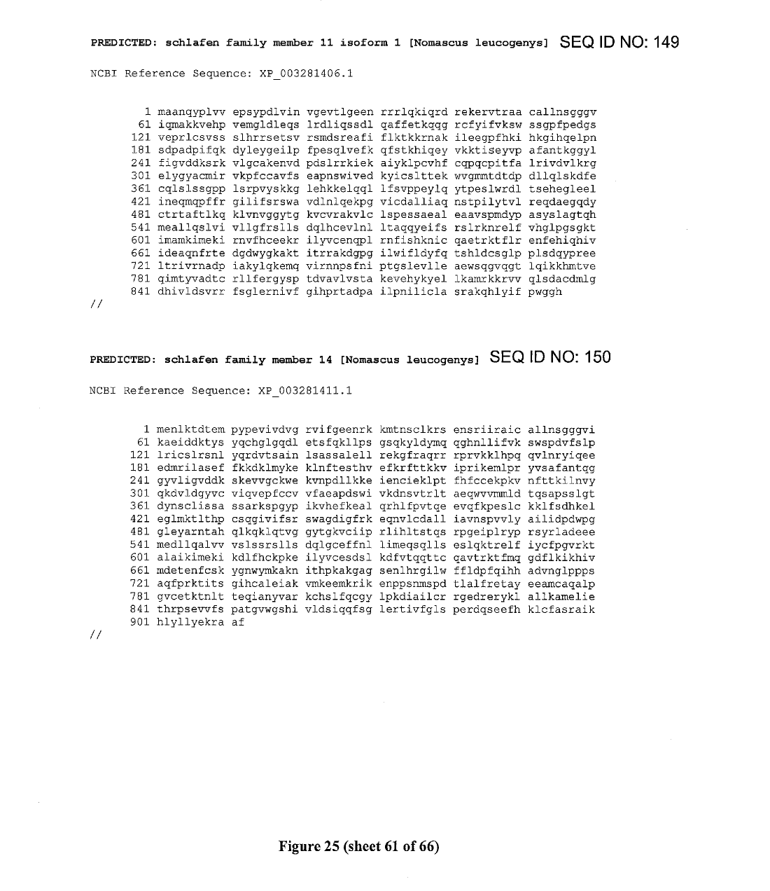

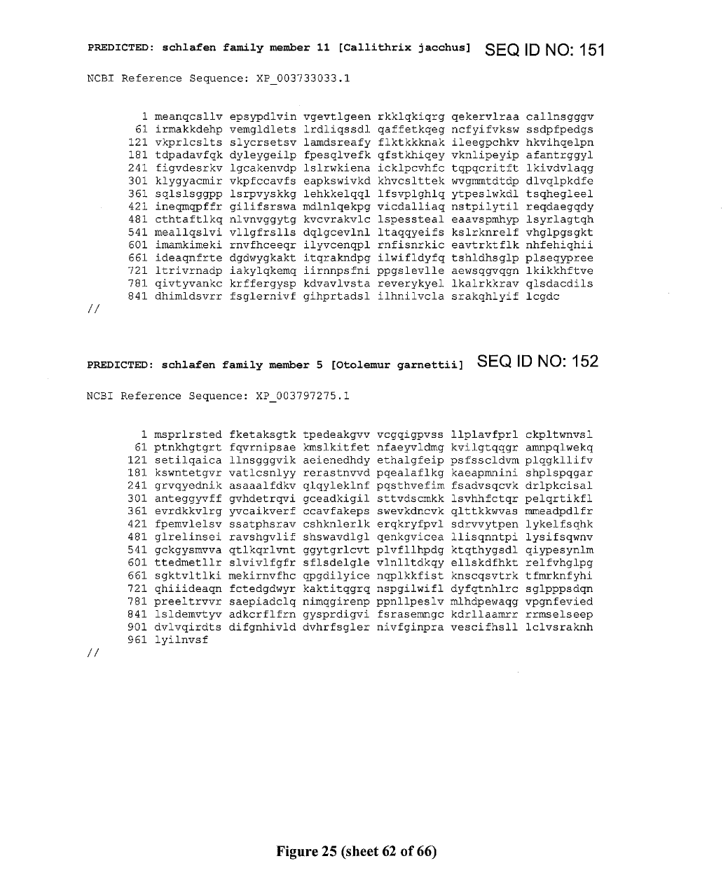

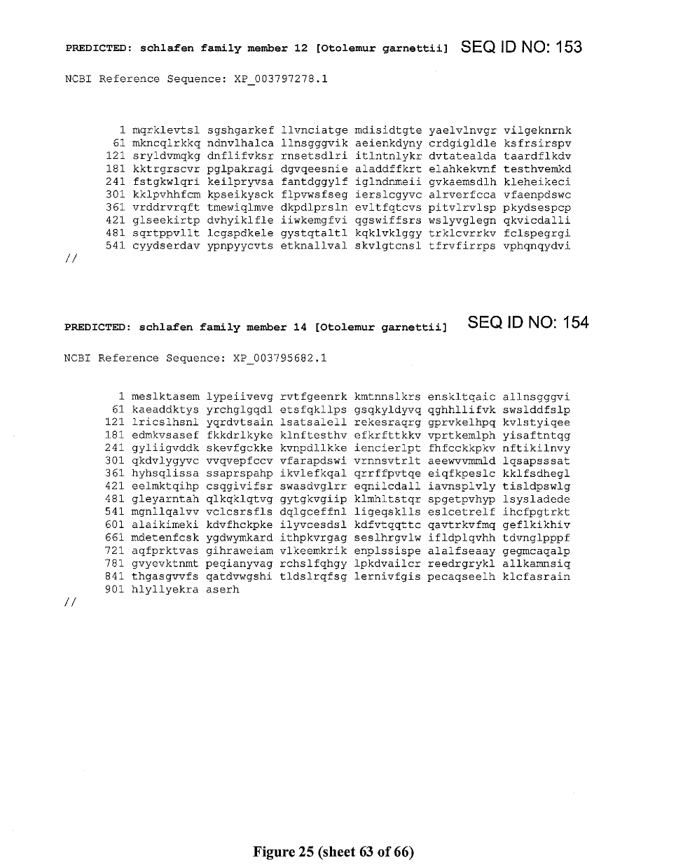

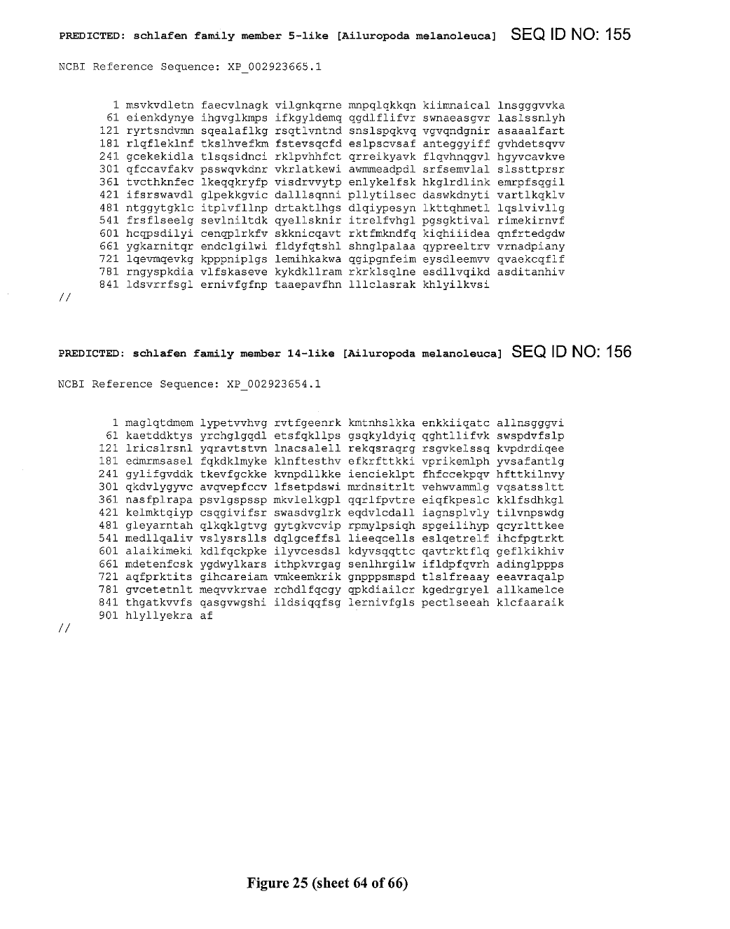

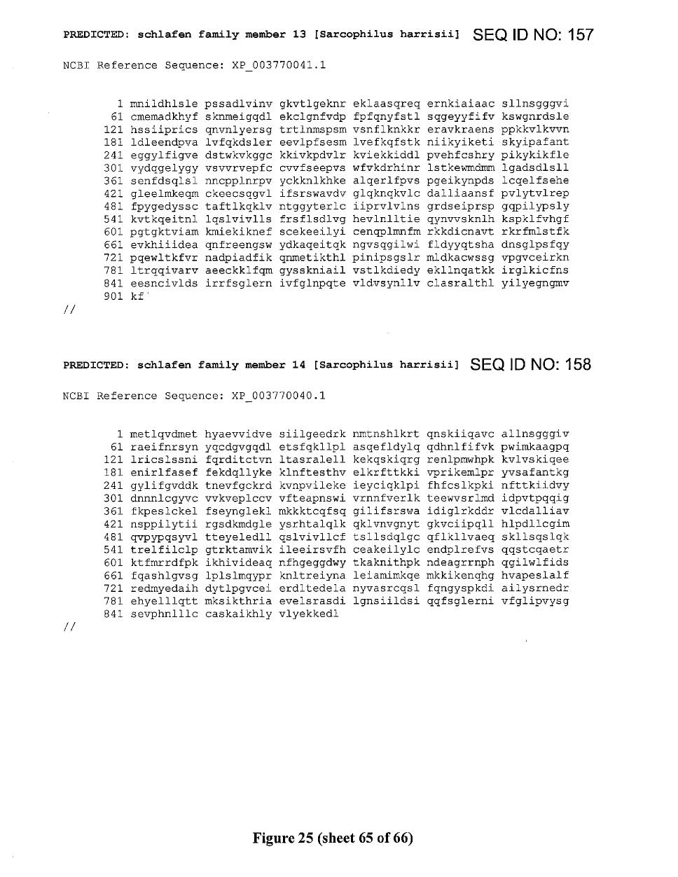

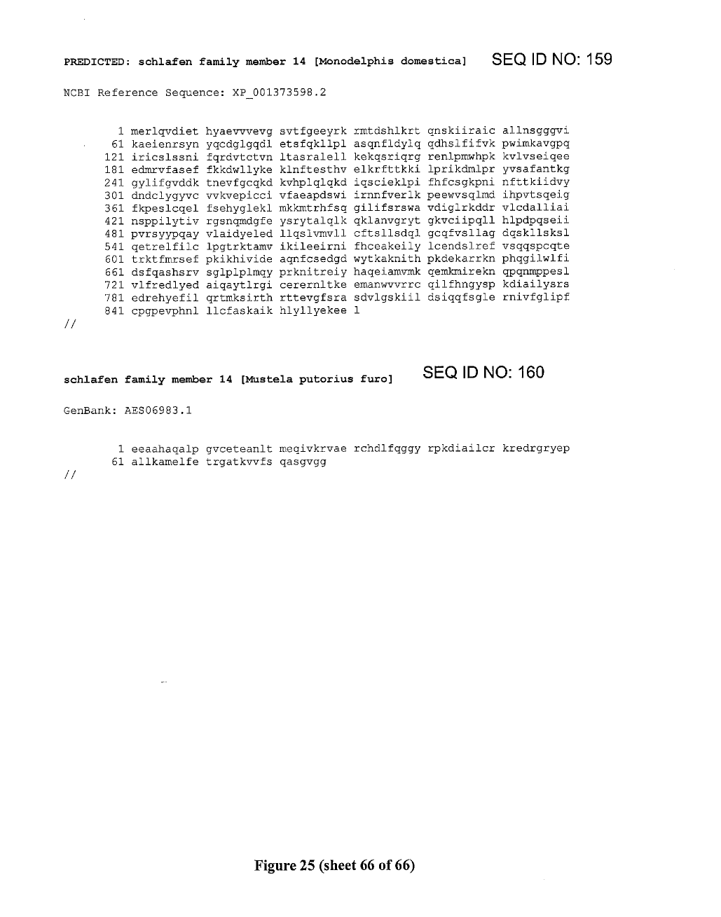

The invention's compositions and/or methods may comprise using Schlafen (Slfn). “Schlafen” and “Slfn” interchangeably refer to one or more proteins that are encoded by the Schlafen (Slfn) genes, which are a subset of Interferon Stimulated early response Genes (ISGs), and which are also directly induced by pathogens via the Interferon Regulatory Factor 3 (IRF3) pathway (Sohn, W. J. et al. Novel transcriptional regulation of the schlafen-2 gene in macrophages in response to TLR-triggered stimulation. Mol Immunol 44, 3273-3282 (2007)). Slfn genes encode a family of Slfn proteins limited to mammalian organisms, and are exemplified by the proteins (such as, but not limited to, Slfn1, Slfn2, Slfn3, Slfn4, Slfn5, Slfn8, Slfn9, Slfn10, Slfn11, Slfn12, Slfn12L, Slfn13, and Slfn14) in Table 1 and FIG. 25.

TABLE 1
Exemplary mammalian Schlafen (Slfn) for use in any one or more of the
invention's compositions and/or methods

| Organism | Slfn | GenBank Protein Accession No. |
|---|---|---|
| Homo sapiens | 5 | AAI25202.1; AAH21238.1; NP_659412.3; Q08AF3.1; AAI25201.1; |
| | | CAD80167.1; ABM87182.1; ABM83860.1 |
| | 11 | Q7Z7L1.2; NP_001098060.1; NP_001098058.1; NP_689483.3; |
| | | NP_001098059.1; NP_001098057.1; AAH52586.1; AAI41661.1; |
| | | CAL38551.1 |
| | 12 | NP_060512.3; AAH35605.1; Q8IYM2.2; NP_060512.3; |
| | | AAH35605.1 |
| | 12L | Q6IEE8.3; NP_001182719.1 |
| | 13 | NP_653283.3; AAI36623.1; AAI71771.1; Q68D06.1; CAL37815.1 |
| | 14 | P0C7P3.2; NP_001123292.1; AAI57878.1; AAI57880.1; |
| | | AAI40848.1; |
| Bos taurus | 3 | DAA19092.1; DAA19091.1 |
| | 11 | XP_002695699.1; XP_600064.2; |
| | 14 | NP_001192831.1 |
| Equus caballus | 5 | XP_001501499.3 |
| | 11 | XP_001501460.2 |
| | 14 | XP_001503985.2 |
| Sus scrofa | 11 | XP_003358204.1 |
| | 14 | XP_003358199.2 |
| Canis lupus | 5 | XP_003435343.1 |
| | 14 | XP_548260.3 |
| Mus musculus | 1 | NP_035537.1; EDL15691.1; AAH52869.1; AAC83825.1; |
| | | CAI25056.1 |
| | 2 | EDL15690.1; AAI38751.1; AAI38750.1; AAC83826.1; CAI25506.1; |
| | 3 | NP_035539.1; AAI45135.1; AAI38071.1; AAI32628.1; |
| | | AAC83827.1; CAI25417.1 |
| | 4 | AAH46511.1; NP_035540.2; AAI50771.1; AAH44865.1; |
| | | AAC83832.1; AAC83833.1; AAC83831.1; CAI25057.1 |
| | 5 | NP_899024.3; EDL15686.1; EDL15687.1; Q8CBA2.2; AAI17910.1; |
| | | AAI17911.1; AAP30074.1; AAP30075.1; |
| | 8 | AAH24709.1; NP_853523.2; NP_001161215.1; AAP30069.1; |
| | | AAP30068.1; CAI25502.1; CAI25503.1; CAI25501.1; |
| | 9 | NP_766384.2; AAP30071.1; AAP30070.1; AAI48385.1 |
| | 10 | AAI15829.1; NP_853520.3; AAP30073.1; AAP30072.1; AAI41536.1 |
| | 12L | NP_035538.1 |
| | 14 | NP_001159500.1 |
| Rattus | 1 | NP_035537.1 |
| norvegicus | | |
| | 3 | NP_446139.1; EDM05466.1; EDM05467.1 |
| | 5 | XP_001081036.1; XP_220775.3 |
| | 8 | EDM05463.1; AAH85777.1; |
| | 12 | XP_002724574.1; XP_001068751.2 |
| | 12L | NP_001100501.1; XP_577117.4; XP_001081053.3 |
| | 13 | NP_001013992.1; |
| | 14 | XP_002727830.2; XP_002724575.2; |
| Cricetulus | 5 | XP_003495874.1 |
| griseus | | |
| Pan | 5 | XP_523607.2 |
| troglodytes | | |
| | 11 | XP_001174320.1; XP_001174307.1; XP_001174334.1; |
| | | XP_001174314.1; XP_001174293.1 |
| | 12 | XP_001174271.1; XP_001174240.1; XP_523608.2 |
| | 12L | XP_001156165.2 |
| | 13 | XP_001174230.2 |
| | 14 | XP_511414.1 |
| Pan paniscus | 5 | XP_003818037.1 |
| | 11 | XP_003818036.1 |
| | 12 | XP_003818034.1; XP_003818035.1; |
| | 12L | XP_003818032.1 |
| | 13 | XP_003818033.1 |
| | 14 | XP_003818077.1 |
| Macaca | 5 | AFH31302.1; AFH31300.1; AFH31298.1; AFH31296.1; |
| mullata | | AFH31301.1; AFH31299.1; AFH31297.1; AFE76936.1; |
| | 11 | XP_001114006.2; |
| | 13 | AFH31205.1; AFE76823.1; AFE76824.1; XP_001114211.1 |
| | 14 | XP_001112128.1 |
| Pongo abelii | 5 | XP_002827306.2 |
| | 11 | XP_002827307.1 |
| | 12 | XP_003778840.1; XP_002827309.1 |
| | 12L | NP_001129013.1; Q5RCZ8.1 |
| | 13 | XP_003778842.1; XP_003778841.1 |
| | 14 | XP_002827334.1; |
| Nomascus | 5 | XP_003281405.1 |
| leucogenys | | |
| | 11 | XP_003281409.1; XP_003281407.1; XP_003281410.1; |
| | | XP_003281408.1; XP_003281406.1; |
| | 14 | XP_003281411.1 |
| Callithrix | 11 | XP_003733033.1 |
| jacchus | | |
| Otolemur | 5 | XP_003797275.1 |
| garnetti | | |
| | 12 | XP_003797278.1 |
| | 14 | XP_003795682.1 |
| Ailuropoda | 5 | XP_002923665.1 |
| melanoleuca | | |
| | 14 | XP_002923654.1 |
| Sarcophilus | 13 | XP_003770041.1 |
| harrisii | | |
| | 14 | XP_003770040.1 |
| Monodelphis | 14 | XP_001373598.2 |
| domestica | | |
| Mustela | 14 | AES06983.1 |
| putorius furo | | |

Nine murine and six human Slfn genes share a conserved NH2-terminus containing a putative AAA-domain, and long Slfns possess motifs resembling DNA/RNA helicase domains, a trait they share with the nucleic acid sensors RIG-I and MDA-5 (Yoneyama et al., J Immunol 175, 2851-2858, doi:175/5/2851 (pii) (2005)). Beyond that, Slfn proteins harbor no sequence similarity to other proteins. In vivo, short and long murine Slfns (muSlfns) inhibit T-cell development (Schwarz, et al., Immunity 9, 657-668, doi:S1074-7613(00)80663-9 (pii) (1998); Gesericket et al., Int Immunol 16, 1535-1548 (2004); Berger et al., Nat Immunol 11, 335-343, doi:ni.1847 (pii) 10.1038/ni.1847 (2010)), and levels of muSlfns are elevated after infection with Brucella or Listeria (Gesericket et al., 2004). However, many ISGs are of unknown or incompletely understood function.

In one embodiment, the invention contemplates a Schlafen protein sequence (and/or portion thereof) that has “at least 85% amino acid sequence identity” to any one or more of the Slfn proteins of Table 1 and FIG. 25, including, for example, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, and at least and 99% to any one or more of the Slfn proteins of Table 1 and FIG. 25.

In one embodiment, the invention contemplates “portion of Schlafen” sequence. The term “portion” of an amino acid sequence (as in a “portion of Schlafen” sequence) refers to fragments of that amino acid sequence. The fragments may range in size from an exemplary 50 contiguous amino acid residues to the entire amino acid sequence minus one amino acid residue. Thus, a polypeptide sequence comprising “at least a portion” of an amino acid sequence” comprises from 50 contiguous amino acid residues of the amino acid sequence to the entire amino acid sequence. In a preferred embodiment, the portion of Schlafen sequence is from amino acid 1 to 579 of any one or more of the Slfn proteins of Table 1 and FIG. 25. In another embodiment, the portion of Slfn sequence is at least 50-amino acids long and is comprised by the sequence from amino acid 1 to 579 of any one or more of the Slfn proteins of Table 1 and FIG. 25.

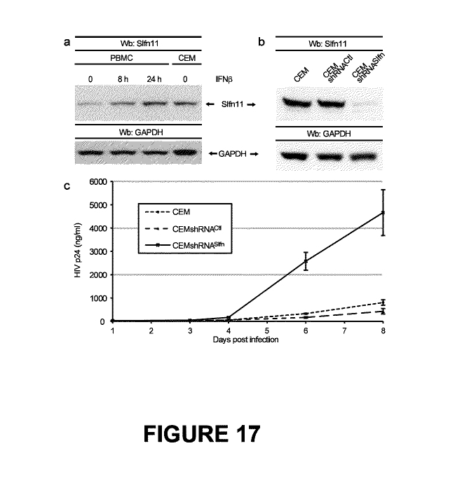

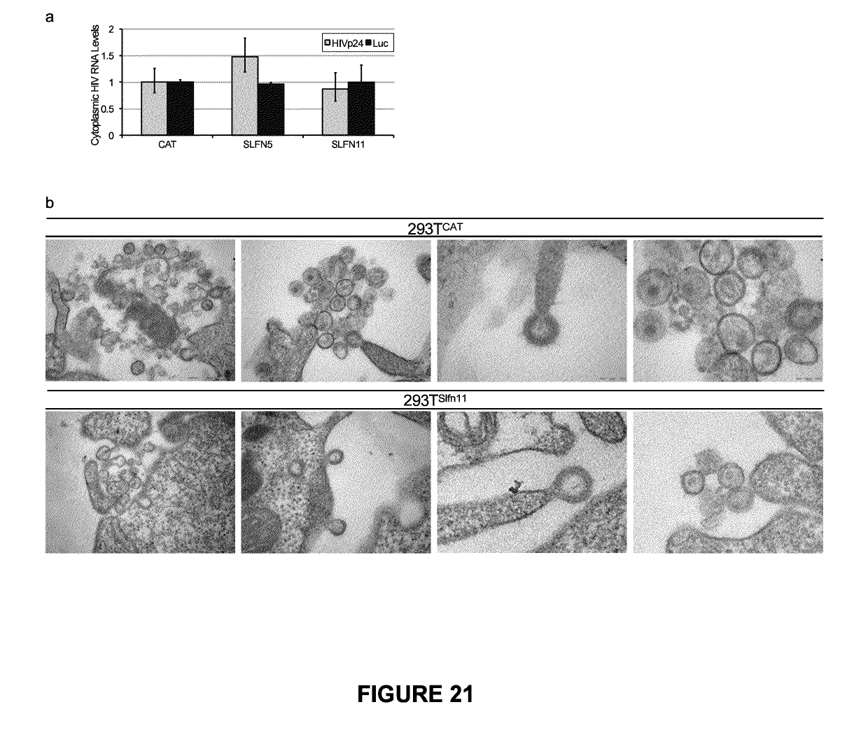

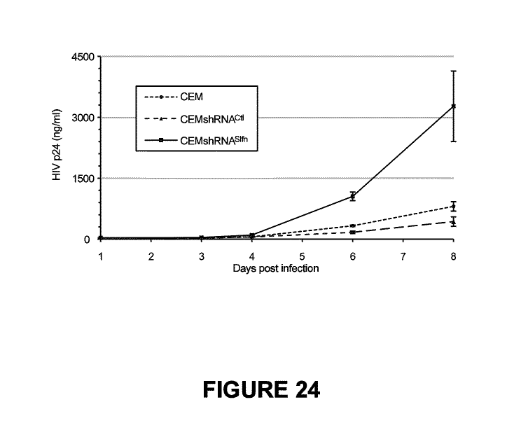

In one preferred embodiment, the invention contemplates Slfn that comprise Slfn11. In one embodiment, the Slfn11 comprises human Slfn11. Data herein demonstrate that the exemplary huSlfn11 reduced the formation of viral particles inside the cells (FIG. 21b). Data herein further demonstrate that the anti-retroviral effects of huSlfn11 were not limited to the exemplary retroviruses and cells used herein (Example 15, FIGS. 17c and 24).

In another embodiment, the Slfn11 comprises mouse Slfn11.

### 11. Target Cell

The invention's compositions and/or methods may comprise using a cell. A “cell” that may be used in any one or more of the invention's compositions and/or methods includes, without limitation, permissive cells, transgenic cells, wild type cells, mammalian cells, human cells, non-human cells, primary cells, cell line cells, and cells that are capable of generating an animal.

Thus, the invention's compositions and/or methods may comprise using a cell that is permissive to a virus. These cells are useful in, for example, optionally measuring the effect of the one or more candidate compounds on virus infection of the cell.

The invention's compositions and/or methods may comprise using a cell that is transgenic. In some embodiments, the transgenic cell comprises Slfn that is heterologous to the cell. In some embodiments, the transgenic cell is a 293T cell and the heterologous Slfn is exemplified by one or more of huSlfn5, huSlfn11 (amino acid 1 to amino acid 901), huSlfn11-N (amino acid 1 to amino acid 579) and huSlfn11-C (amino acid 523 to amino acid 901)). The transgenic cells are exemplified by 293T cells described herein that were transfected with huSlfn5, huSlfn11 (amino acid 1-901), huSlfn11-N (amino acid 1-579) or huSlfn11-C (amino acid 523-901) (FIG. 14g).

The invention's compositions and/or methods may comprise using a cell that is wild-type, such as a cell that expresses endogenous Slfn.

The invention's compositions and/or methods may comprise using a human cell, as exemplified, but not limited to, U937 cells (macrophage), ATCC# crl 1593.2; A-375 cells (melanoma/melanocyte), ATCC# crl-1619; KLE cells (uterine endometrium), ATCC# crl-1622; T98G cells (glioblastoma), ATCC# crl-1690; CCF-STTG1 cells (astrocytoma), ATCC# crl-1718; HUV-EC-C cells (vascular endothelium), ATCC# CRL-1730; UM-UC-3 cells (bladder), ATCC# crl-1749; CCD841-CoN cells (colon, ATCC# crl-1790; SNU-423 cells (hepatocellular carcinoma), ATCC# crl-2238; WI38 cells (lung, normal), ATCC# crl-75; Raji cells (lymphoblastoid), ATCC# ccl-86; BeWo cells (placenta, choriocarcinoma), ATCC# eel-98; HT1080 cells (fibrosarcoma), ATCC# ccl-121; MIA PaCa2 cells (pancreas), ATCC# crl-1420; CCD-25SK cells (skin fibroblast), ATCC# crl-1474; ZR75-30 cells (mammary gland), ATCC# crl-1504; HOS cells (bone osteosarcoma), ATCC# crl-1543; 293-SF cells (kidney), ATCC# crl-1573; LL47 (MaDo) cells (normal lymphoblast), ATCC# ccl-135; and HeLa cells (cervical carcinoma), ATCC# ccl-2.

The invention's compositions and/or methods may comprise using a non-human cell, as exemplified by, but not limited to, LM cells (mouse fibroblast), ATCC# ccl-1.2; NCTC 3526 cells (rhesus monkey kidney), ATCC# ccl-7.2; BHK-21 cells (golden hamster kidney), ATCC# ccl-10; MDBK cells (bovine kidney), ATCC# ccl-22; PK 15 cells (pig kidney), ATCC# ccl-33; MDCK cells (dog kidney), ATCC# ccl-34; PtK1 cells (kangaroo rat kidney), ATCC# ccl-35; Rk 13 cells (rabbit kidney), ATCC# ccl-37; Dede cells (Chinese hamster lung fibroblast), ATCC# ccl-39; Bu (IMR31) cells (bison lung fibroblast), ATCC# ccl-40; FHM cells (minnow epithelial), ATCC# ccl-42; LC-540 cells (rat Leydig cell tumor), ATCC# ccl-43; TH-1 cells (turtle heart epithelial), ATCC# ccl-50; E. Derm (NBL-6) cells (horse fibroblast), ATCC# ccl-57; MvLn cells (mink epithelial), ATCC# ccl-64; Ch1 Es cells (goat fibroblast), ATCC# ccl-73; Pl I Nt cells (raccoon fibroblast), ATCC# ccl-74; Sp I k cells (dolphin epithelial), ATCC# ccl-78; CRFK cells (cat epithelial), ATCC# ccl-94; Gekko Lung 1 cells (lizard-gekko epithelial), ATCC# ccl-111; Aedes Aegypti cells (mosquito epithelial), ATCC# ccl-125; ICR 134 cells (frog epithelial), ATCC# ccl-128; Duck embryo cells (duck fibroblast), ATCC# ccl-141; and DBS Fcl-1 cells (monkey lung fibroblast), ATCC# ccl-161.

### 12. Virus

The invention's compositions and/or methods may comprise using a virus. The term “virus” refers to obligate, ultramicroscopic, intracellular parasites incapable of autonomous replication (i.e., replication requires the use of the host cell's machinery). The term virus includes retroviruses and DNA viruses.

The invention's compositions and/or methods may comprise using a retrovirus. “Retrovirus” is a small enveloped RNA virus, containing two identical single stranded positive sense RNA genomes enclosed in an enveloped capsid. Retroviruses have an enzyme, called reverse transcriptase that gives them the unique property of transcribing their RNA into DNA after entering a cell. The retroviral DNA can then integrate into the chromosomal DNA of the host cell, to be expressed there. Retroviruses have a genome flanked by Long Terminal Repeats (LTR) and 4 main genes gag, pol, pro and env. Retroviruses are exemplified by lentivirus (such as “Human Immunodeficiency Virus” (“HIV”), Simian Immunodeficiency Virus, Feline Immunodeficiency Virus), baculovirus, “murine stem cell virus” (“MSCV”), simian foamy virus, avian leukosis virus, Rous sarcoma virus, mouse mammary tumor virus, murine leukemia virus, feline leukemia virus, bovine leukemia virus, human T-lymphotropic virus, walleye dermal sarcoma virus.

In a preferred embodiment, the retrovirus comprises HIV. Data herein demonstrate inhibition of HIV particle formation and/or release (FIG. 14a).

In another preferred embodiment, the retrovirus comprises murine stem cell virus (MSCV). Data herein demonstrate inhibition of MSCV particle formation and/or release (FIG. 20b).

The invention's compositions and/or methods may comprise using a DNA virus. “DNA virus” is a virus in which the genetic material is DNA rather than RNA, and are exemplified by Arenaviridae, Baculoviridae, Birnaviridae, Bunyaviridae, Cardiovirus, Corticoviridae, Cystoviridae, Epstein-Barr virus, Filoviridae, Hepadnviridae, Hepatitis virus, Herpesviridae, Influenza virus, Inoviridae, Iridoviridae, Metapneumovirus, Orthomyxoviridae, Papovavirus, Paramyxoviridae, Parvoviridae, Polydnaviridae, Poxyviridae, Reoviridae, Rhabdoviridae, Semliki Forest virus, Tetraviridae, Toroviridae, Vaccinia virus, Vesicular stomatitis virus. The DNA may be double-stranded or single-stranded. Double-stranded DNA viruses (class I viruses) include adenoviruses, “adeno-associated virus” (“AAV”), herpes viruses, and poxviruses. Single-stranded DNA viruses (class II viruses) include parvoviruses and coliphages.

### 13. Candidate Compound

The invention's compositions and/or methods may comprise using a candidate compound. The terms “candidate compound,” “test compound,” “compound,” “agent,” “test agent,” “molecule,” and “test molecule,” as used herein, interchangeably refer to any type of molecule (for example, a peptide, polypeptide, vaccine, antibody, nucleic acid, nucleic acid sequence, carbohydrate, saccharide, polysaccharide, lipid, organic molecule, inorganic molecule, etc.) obtained from any source (for example, plant, animal, and environmental source, etc.), or prepared by any method (for example, purification of naturally occurring molecules, chemical synthesis, genetic engineering, etc.).

A candidate compound may have a known, or unknown, structure and/or composition. Examples of candidate compounds that have unknown compositions include cell extracts, tissue extracts, growth medium in which prokaryotic, eukaryotic, and archaebacterial cells have been cultured, fermentation broths, protein expression libraries, DNA libraries, and the like.

The “candidate compound,” can be synthetic, naturally occurring, or a combination thereof. A synthetic candidate compound can be a member of a library of candidate compounds (e.g., a combinatorial chemical library). Methods for making these libraries of compounds are known in the art, such as methods for preparing oligonucleotide libraries (Gold et al., U.S. Pat. No. 5,270,163, incorporated by reference); peptide libraries (Koivunen et al. J. Cell Biol., 124: 373-380 (1994)); peptidomimetic libraries (Blondelle et al., Trends Anal. Chem. 14:83-92 (1995)) oligosaccharide libraries (York et al., Carb. Res. 285:99-128 (1996); Liang et al., Science 274:1520-1522 (1996); and Ding et al., Adv. Expt. Med. Biol. 376:261-269 (1995)); lipoprotein libraries (de Kruif et al., FEBS Lett., 399:232-236 (1996)); glycoprotein or glycolipid libraries (Karaoglu et al., J. Cell Biol. 130:567-577 (1995)); or chemical libraries containing, for example, drugs or other pharmaceutical agents (Gordon et al., J. Med. Chem. 37:1385-1401 (1994); Ecker and Crook, Bio/Technology 13:351-360 (1995), U.S. Pat. No. 5,760,029, incorporated by reference). Additional examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

A synthetic candidate compound may be a member of a biological library or peptoid library (i.e., library of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994)). The biological library and peptoid library are particularly suited for use with peptide libraries.

In addition, a synthetic candidate compound may be a member of a spatially addressable parallel solid phase library or solution phase library, of a synthetic library that uses methods such as a deconvolution method, a ‘one-bead one-compound’ library method, affinity chromatography selection. These methods are particularly suited to peptide libraries, non-peptide oligomer libraries, and small molecule libraries of compounds (Lam (1997) Anticancer Drug Des. 12:145).

Libraries of diverse molecules also can be obtained from commercial sources, e.g., Brandon Associates (Merrimakc, N.H.) and Aldrich Chemical Co (Milwaukee, Wis.).

A naturally occurring candidate compound can be a component of a cellular extract or bodily fluid (e.g., urine, blood, tears, sweat, or saliva). A naturally occurring candidate compound may be obtained by extraction and/or purification of commercially available libraries of bacterial, fungal, plant, and animal extracts.

A candidate compound includes both known therapeutic compounds, and potentially therapeutic compounds. An agent can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

A candidate compound includes one or more amino acid sequence, exemplified by, but not limited to a) Slfn sequence (exemplified in Table 1 and FIG. 25), b) portion of Slfn sequence (e.g., amino acid 1 to 579 of any one or more of the Schlafen sequences of Table 1 and FIG. 25), c) amino acid sequence having at least 85% amino acid sequence identity to the Slfn sequence (exemplified in Table 1 and FIG. 25), and d) amino acid sequence having at least 85% amino acid sequence identity to the portion of a Slfn sequence.

Candidate compounds include an “RNA interference sequence” (also referred to as “RNAi sequence”) which refers to an small hairpin RNA (shRNA) sequence, RNAi sequence and/or small interfering RNA (siRNA) sequence that specifically binds to a target mRNA sequence (such as to Slfn mRNA sequence). RNA interference is the process of sequence-specific, post-transcriptional gene silencing in animals and plants, initiated by shRNA and/or siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs. Carthew (2001) has reported (Curr. Opin. Cell Biol. 13 (2):244-248) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

Thus, in one embodiment, one or more candidate compounds comprise an RNA interference (RNAi) sequence that specifically binds to an mRNA sequence that encodes one or more amino acid sequences exemplified by one or more of a) Slfn sequence (exemplified in Table 1 and FIG. 25), b) portion of Slfn sequence (e.g., amino acid 1 to 579 of any one or more of the Schlafen sequences of Table 1 and FIG. 25), c) amino acid sequence having at least 85% amino acid sequence identity to the Slfn sequence (exemplified in Table 1 and FIG. 25), and d) amino acid sequence having at least 85% amino acid sequence identity to the portion of Slfn sequence. In particular embodiments, the RNA interference sequence is exemplified by one or more of short hairpin RNA (shRNA) sequence, short interfering RNA (siRNA) sequence, and hairpin RNA (hpRNA) sequence.

Candidate compounds include shRNA. As used herein, the term “shRNA” or “short hairpin RNA” refers to a sequence of ribonucleotides comprising a single-stranded RNA polymer that makes a tight hairpin turn on itself to provide a “double-stranded” or duplexed region. shRNA can be used to silence gene expression via RNA interference. shRNA hairpin is cleaved into short interfering RNAs (siRNA) by the cellular machinery and then bound to the RNA-induced silencing complex (RISC). It is believed that the complex inhibits RNA as a consequence of the complexed siRNA hybridizing to and cleaving RNAs that match the siRNA that is bound thereto. Methods for designing an shRNA sequence are known in the art including web-based software BLOCK-iT™ RNAi Designer. The BLOCK-iT™ RNAi Designer software may be used to design siRNA, Stealth RNAi™ siRNA, miR RNAi inserts and shRNA inserts for any target sequence. In one embodiment, C43 targeting sequences were further prioritized based on guidelines described by Ui-Tei et al. (Ui-Tei et al. Nucleic Acids Res., 32, 936-948). These criteria were: i. A/U at the 5′ end of the antisense strand; ii. G/C at the 5′ end of the sense strand; iii. AU-richness in the 5′ terminal one-third of the antisense strand; and iv. The absence of any GC stretch over 9 bp in length.

Candidate compounds include siRNA. The term “siRNA” refers to short interfering RNA. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

Candidate compounds include hpRNA. The terms “hpRNA” and “hairpin RNA” refer to self-complementary RNA that forms hairpin loops and functions to silence genes (e.g. Wesley et al. (2001) The Plant Journal 27 (6):581-590; herein incorporated by reference). The term “ihpRNA” refers to intron-spliced hpRNA that functions to silence genes.

Candidate compounds include target RNA molecules. The term “target RNA molecule” refers to an RNA molecule to which at least one strand of the short double-stranded region of a siRNA is homologous or complementary. Typically, when such homology or complementarity is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

### EXPERIMENTAL

### Example 1

### a) Significance

Innate immune recognition is mediated by a system of germline-encoded receptors that recognize conserved pathogen-associated molecular patterns (PAMPs) (2). These pattern recognition receptors, or PRRs, are coupled to signaling cascades that mediate the induction of immune-response genes, and represent the most ancient host defense system in mammals, insects and plants. In mammals, one of the most pronounced consequences of activation of these sensors by viruses is the induction of type I IFN, a cytokine with potent antiviral activity. The mechanism by which IFNs activate gene expression has been the target of intense research efforts for many years. Binding of the type I IFNs, IFNα/β, to their specific cell surface receptors IFNAR1 and IFNAR2 leads to the tyrosine phosphorylation and activation of several members of the Signal Transducer and Activator of Transcription (STAT) family of transcription factors (3). Among those, STAT1, STAT2 and IRF9 cooperatively form the DNA binding protein complex ISGF3 which is required for upregulation of interferon stimulated genes (ISGs) through an Interferon Stimulated Response Element (ISRE). Numerous ISGs represent components of the antiviral defense such as the 2′-5′ oligoadenylate synthetase (4) and the dsRNA activated protein kinase (PKR) (5), cell surface proteins such as ICAM (6) or the MHC class I and II molecules (7), however, many other ISRE containing genes are of unknown or not well understood function.

Perhaps not surprisingly, since many ISGs have antiviral activity, viruses—including retroviruses such as HIV—evolved to suppress IFN induction or function. The existence of these viral countermeasures, coupled with the fact that multiple type 1 IFN-induced factors can restrict virus replication, serve to underscore the importance of innate cellular defense mechanisms in limiting, amongst others, infection by HIV. Despite the recognized antiviral activity of type I IFN, only a few specific factors that mediate the inhibitory effect of IFN on retrovirus replication have been identified. These include: TRIM5α which blocks HIV-1 infection at the reverse transcription stage (12-15); TRIM22 which blocks HIV-1 budding (16); TRIM31 which restricts an early step of HIV-1 replication (17, 18); APOBEC3G and 3F which act during reverse transcription by both cytidine deaminase-dependent and -independent mechanisms, and are counteracted by the HIV-1 accessory protein Vif (19-23); Tetherin/BST-2 which blocks viral budding and release (24, 25); ISG15 which blocks the Gag-Tsg101 interaction important for HIV-1 budding (26); and ISG20, a 3′-5′ exoribonuclease which can limit HIV-1 infection (27). However, little is known about cellular restriction factors that inhibit virus production (which is also evidenced by the absence of antiviral drugs acting at this point in the viral replication cycle).

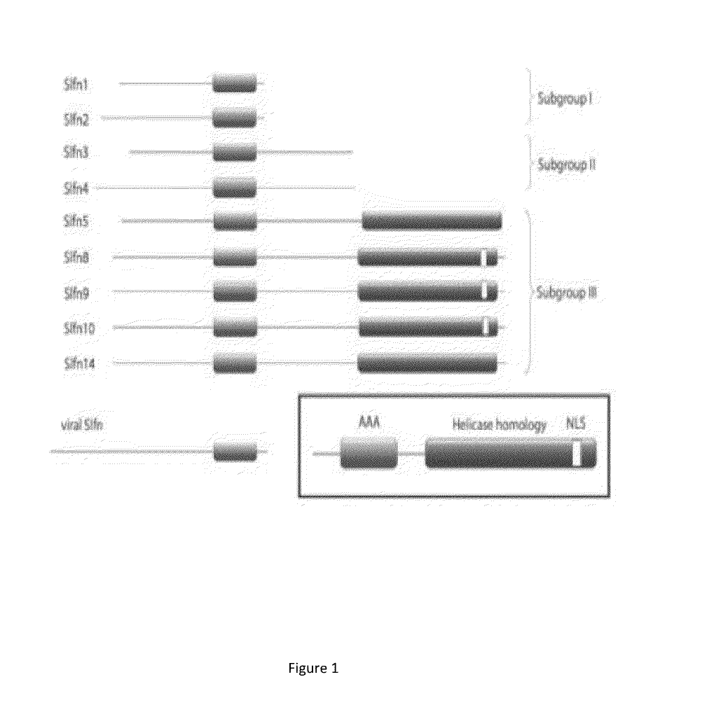

Among the ISGs of unknown function are the Slfn genes, which encode a family of proteins that includes at least 10 distinct members in the mouse and 6 in humans. Slfns can be categorized into three distinct groups based on the length of the C terminus: short forms (muSlfn1 and muSlfn2), intermediate forms (muSlfn3 and 4), and long forms (muSlfn5, 8, 9, and 10), however, humans lack homologs to the short muSlfn1 and muSlfn2 isoforms (see FIG. 1). All Slfn proteins share a conserved NH2-terminal region that contains a putative divergent ATPase (AAA) domain, whereas long form Slfns possess additional short motifs that resemble domains found in members of the DNA/RNA helicase superfamily. In vivo, ectopically expressed short and long muSlfns inhibit T-cell development (8, 9), whereas in vitro, only muSlfn1 caused arrest of fibroblast growth by inhibition of cyclin D1 (8-10). Additionally, the expression levels of muSlfns were elevated after infection with the intracellular pathogens Brucella (11) and Listeria (9).

Only few publications exist on Slfn proteins, and besides their somewhat controversial inhibitory effect on cell cycle progression, very little is known about the molecular mechanism behind their action, or their biological functions. We discovered that expression of huSlfn11 attenuates the production of retroviruses including HIV, whereas abrogation of Slfn11 expression resulted in a significant increase in the release of infectious retrovirus. Slfn11 expression had no effect on early steps of the infection cycle encompassing entry, reverse transcription, integration and transcription, furthermore, we did not observe an inhibition of late stage budding or release of the viral particles. In contrast, our studies revealed a clear inhibition of protein synthesis, which was strikingly restricted to proteins of viral origin. This high degree of selectivity can be traced to the unique codon bias displayed by HIV, which favors A/T-rich codons. This proposal aims to define the role and the mechanism of Slfn proteins, particularly Slfn11, as inhibitors of retrovirus/HIV production.

The studies proposed in this application will for the first time address this novel antiviral function of Slfn proteins on a cellular and molecular level. The results will not only shed light onto a novel area of the innate antiviral response against HIV and other retroviruses (and likely other viruses with similar codon bias, e.g. Influenza), but will also provide the necessary understanding of Slfn protein function required to exploit their prospective diagnostic and therapeutic value.

### (b) Innovation

The innate immune response is an ancient defense system made up of functionally distinct subsystems that have evolved to counter infection by microbial pathogens including retroviruses such as HIV, the causative agent of AIDS. The innate response is generally not antigen-specific, and is composed of the interferon (IFN) system, as well as cell-based anti-pathogen countermeasures that serve to restrict the replication of pathogens early in infection. In addition, a successful innate response promotes the secretion of cytokines and growth factors that shape the later pathogen-specific, or acquired immune response composed of B- and T-lymphocytes. The ability to mount a vigorous innate response and to transition to a highly-specific acquired response is thought to be essential for combating and controlling infection. Compelling evidence suggests that immunological events occurring during the first days and weeks after retroviral infection are critical determinants shaping the course of, for instance, HIV/AIDS disease, and the mechanisms that underlie the failure of innate immune responses to restrict HIV infection are currently under intense investigation (28). Understanding the early innate immune response and the pathways that promote or restrict the kinetics of different steps of HIV infection is essential for devising novel pharmacological strategies for therapeutic intervention during these infectious disease processes, or to prevent infection.

The classical model for HIV infection describes a strong anti-HIV T-cell response early in infection, which results in suppression of virus loads in plasma, however this response is unable to completely clear the infection. The immunological events that occur during the first days and weeks after HIV infection are critical determinants that account for the failure of the later adaptive response to completely clear infection. Particularly innate immune response sensors such as the Toll-like Receptors (TLRs) or RIG-I like receptors (RLRs) involved in HIV infection have been the target of intense study. In contrast, little is known about cellular restriction factors that target the late stages in the viral replication cycle including those governing viral protein synthesis. Our discovery of a Slfn family protein as an executioner of an anti-retroviral innate immune response is completely novel and unique, and our proposed studies are aimed at the characterization of this novel innate immune system component set into motion by IRF-3 activation or type I interferon production.

The main focus of this application is to define the functional domains of Slfn11, determine the requirements for its anti-retroviral activity (e.g. ATP binding, oligomerization, etc.), and elucidate the molecular mechanism by which Slfn11 accomplishes inhibition of (retro)viral protein synthesis. In addition to these studies, we are also interested in determining whether functional homologs to huSlfn11 are present in the mouse. Five muSlfns exist with approximately equal sequence similarity to huSlfn11 (muSlfn 5, 8, 9, 10 & 14) and it is possible that one of these proteins is capable of inhibiting retrovirus production including HIV. In this case, it would be reasonable to theorize that HIV has evolved countermeasures against huSlfn11, which might be ineffective against the muSlfn11 homolog. As such, an unopposed murine Slfn family member could contribute to the block of HIV replication in murine cells, and a better understanding of this process will bring the field one step closer to a mouse model of HIV infection.

### (c) Approach

The first member of the Slfn family, Slfn1, was discovered by a subtractive hybridization between CD4⁺CD8⁺ double-positive and CD4⁺ single-positive T cells (8). Slfns represent a rapidly expanding family of proteins, which now includes at least ten distinct members in the mouse, but no homologs can be found in yeast, C. elegans or Drosophila. Strikingly, the only non-mammalian ‘organisms’ that encode slfn-like proteins are all members of the Pox-viruses. Each Slfn mRNA is encoded by a distinct gene rather than derived from alternative splicing. Murine Slfns can be categorized into three distinct groups based on the length of the C terminus: short forms (Slfn1 and Slfn2), intermediate forms (Slfn3 and 4), and long forms (Slfn5, 8, 9, 10 and 14), however, humans lack the short Slfn-1 and Slfn-2 isoforms. All Slfn proteins share a conserved NH2-terminal region that contains a putative divergent ATPase (AAA+) domain, whereas long Slfns possess several weakly preserved motifs found in members of the DNA/RNA helicase superfamily.

In vivo, short and long m-slfns inhibit T-cell development (8, 9), whereas in vitro, only slfn1 caused arrest of fibroblast growth by inhibition of cyclin D1 (8-10). However, the antiproliferative effects of Slfn proteins have been recently questioned (29). Additionally, the expression level of Slfns is elevated after infection with the intracellular pathogens Brucella (11) and Listeria (9). Overall, only a few publications exist in PubMed on Slfn proteins, and besides their controversial inhibitory effect on cell cycle progression, nothing is known about the molecular mechanism behind their action, or their biological functions.

Our lab has been investigating antiviral gene expression mediated by IRF3 and/or IFN for many years. In order to identify IRF3-dependent regulated genes, we performed a few years ago micro-array expression profiling on TLR-stimulated WT and IRF3^{-/-} peritoneal macrophages. Among the differentially induced genes were several members of the Slfn protein family. Subsequent analysis by qPCR confirmed that Slfn proteins are indeed induced by TLR ligation as well as IFN stimulation.

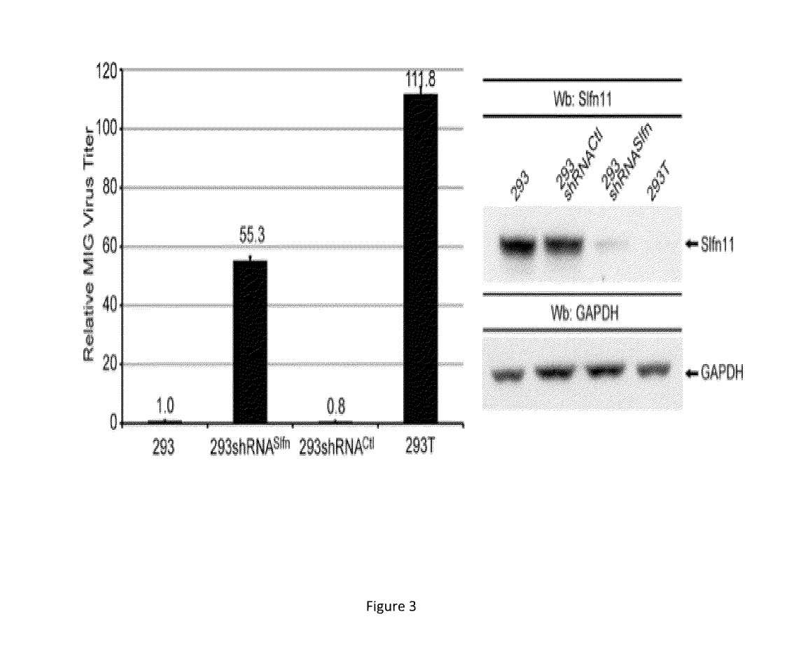

Reconciling the expression pattern results with the facts that Slfns harbor a putative helicase domain similar to RIG-I, we reasoned that Slfn proteins play a role in the innate immune response. As the human genome encodes fewer Slfn proteins than the murine genome, we decided to continue our studies with human cells. The prevailing Slfn in most human cells we tested is huSlfn11. We further observed that huSlfn11 was prominently expressed in HEK293, but not in HEK293T cells. HEK293-related cell lines can be used as packaging cells for the production of retrovirus, and we found that HEK293T cells produce significantly higher viral titers than HEK293 cells (FIG. 3, bars 1 & 4).

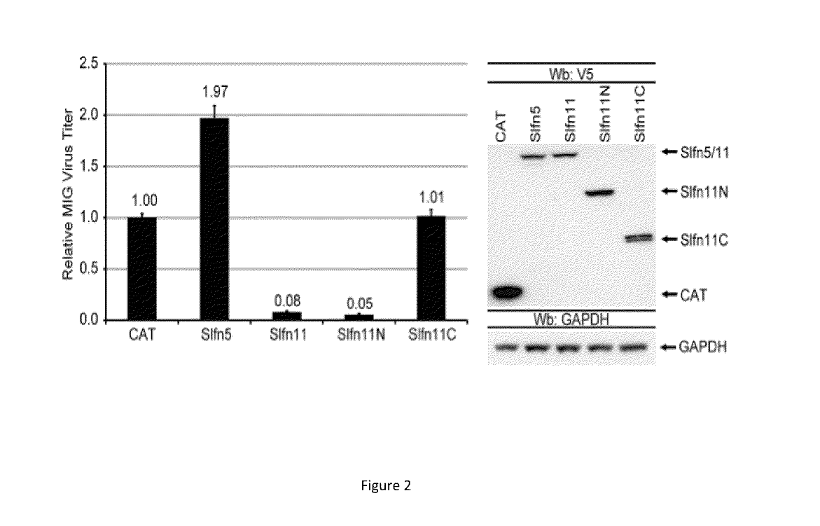

To address whether these differences can be attributed to dissimilar huSlfn11 expression, we ectopically expressed huSlfn11 (aa 1-901) in HEK293T cells, and analyzed their ability to produce ecotropic retrovirus after transfection with the appropriate plasmids. Indeed, huSlfn11, but not huSlfn5, inhibited virus production in 293T cells (FIG. 2). Furthermore, this activity was contained in the NH2-terminal, AAA+ domain-containing region of the huSlfn11 protein (aa 1-579), whereas no effect of the isolated COOH-terminal region (aa 523-901) was observed.

Conversely, we established stable HEK293 cells harboring a huSlfn11-specific shRNA or a non-targeting shRNA as control, and tested their ability to produce ecotropic retrovirus. Abrogation of huSlfn11 expression resulted in a dramatic increase in virus production (FIG. 3, bars 2 and 3).

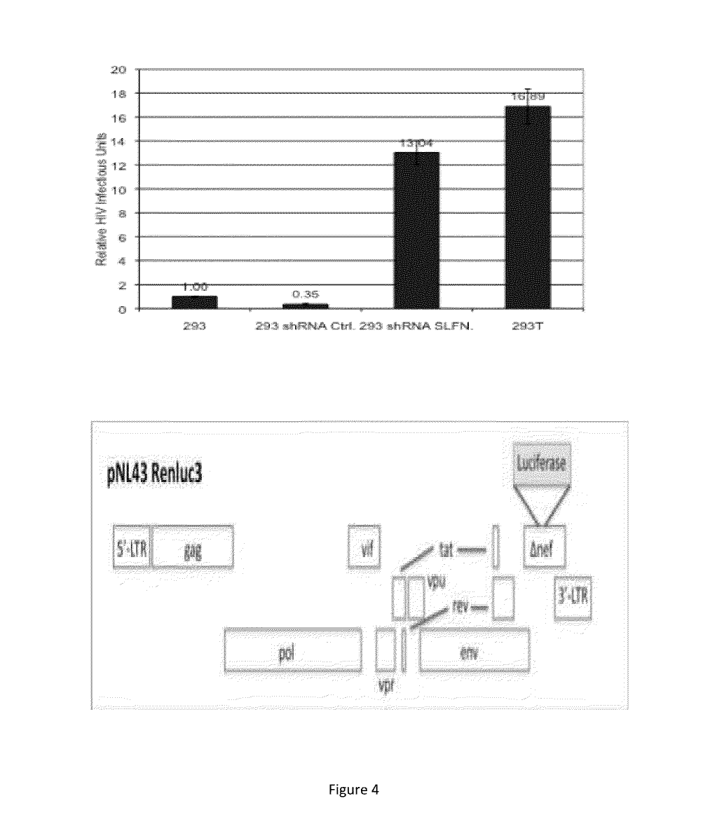

Most importantly, the role of huSlfn11 as viral restriction factor is not limited to this mouse-tropic retrovirus, but similarly affected the production of VSV-pseudotyped HIV. As shown in FIG. 4, abrogation of huSlfn11 expression dramatically increases the amount of infectious HIV particles released from the cells.

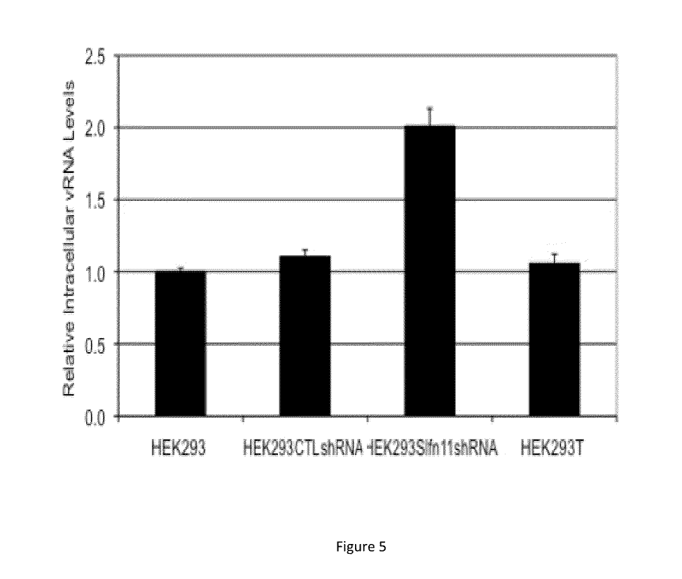

Thus, the abrogation of huSlfn11 expression resulted in a significant increase in the production of infectious retrovirus, whereas overexpression of huSlfn11 attenuated virus production. Further studies confirmed the results obtained from the viral titer analysis by revealing a reduction in extracellular p24 and viral RNA levels in the presence of huSlfn11. The inhibitory effect of huSlfn11 on viral particle concentration in the supernatants was preserved regardless of the presence of viral envelope protein. In contrast, we did not detect a significant difference in intracellular viral RNA levels (see FIG. 5). It should also be noted that we did not observe any induction of ISGs such as ISG15, ISG54 or BST-2 in our experiments.

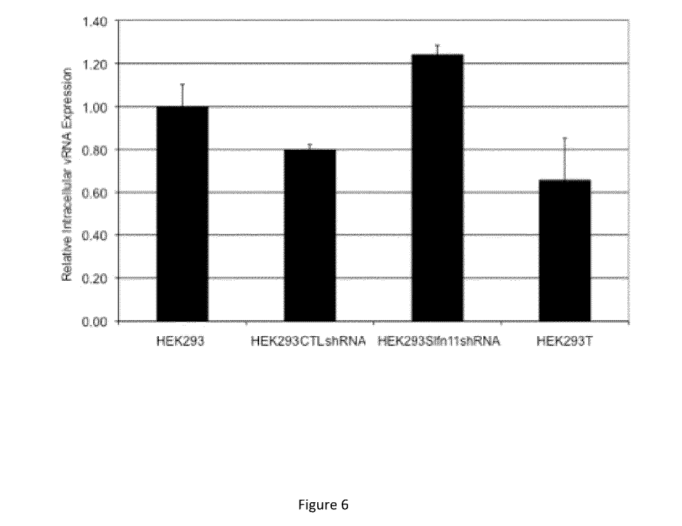

Strikingly, when these cell lines where infected with VSVg-pseudotyped HIV (FIG. 6), or amphitropic MSCV, no differences between the cell lines in the levels of luciferase expression were observed, demonstrating that huSlfn11 inhibits the production of the virus rather than the early stages of infection including entry, RT, integration or transcription (FIG. 6).

This suggested that the function of the huSlfn11 protein as an innate immune response factor is independent of other interferon-induced genes and lies in the very late stages of the infection cycle, possibly in the processes of viral protein translation or assembly. To test whether Slfn11 affects assembly or release, budding or release of HIV particles, we acquired EM images of these events in HEK293T and HEK293T^{Slfn11} cells. These studies illustrated that, once initiated, the budding and release processes were intact in the presence of huSlfn11, but a significantly lower number of such events occurred in the HEK293T^{Slfn11} cells correlating with a >90% reduction in p24 levels in the cell supernatants.

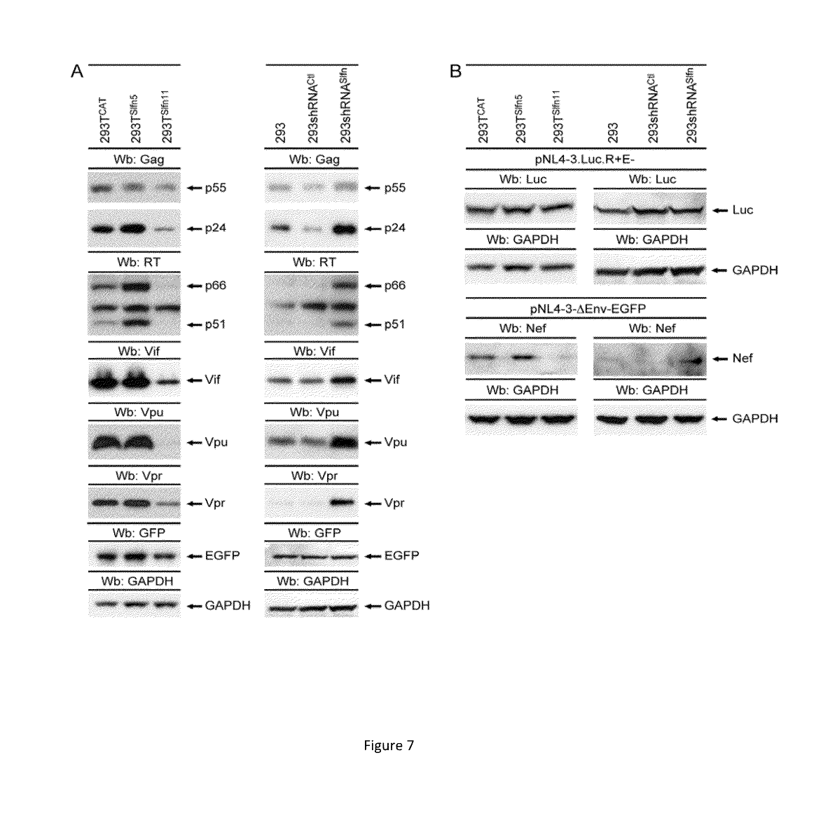

Next, the two cell sets were analyzed for the expression of viral proteins encoded by the retroviral vector pNL4-3.Luc.R⁺E⁻. In striking contrast to the unaltered viral RNA levels, there was a profound effect of huSlfn11 on the amount of the cell-associated p55 Gag precursor protein, and the p24 capsid protein derived through proteolytic cleavage of p55 during the maturation of the virions (FIG. 7A, top). To create a clearer picture of the effect of huSlfn11 on p55 expression, we eliminated its proteolytic processing by introducing a stop-codon into the pro gene in pNL4-3.Luc.R⁺E⁻ to abolish expression of the viral protease. The results showed that p24 could no longer be detected, however, modulation of p55 expression by huSlfn11 was still present. As with gag-derived proteins, huSlfn11 had a striking effect on the intracellular protein levels of reverse transcriptase (RT), Vif, Vpu and Vpr (FIG. 7A). Surprisingly, we did not observe any reduction in the amount of EGFP protein derived from a co-transfected mammalian expression vector, or of GAPDH (FIG. 7, bottom panels), indicating that the limited viral protein production is not the results of a global shutdown of protein synthesis.

Even more surprising, though, was our observation that expression of luciferase, which is encoded in the pNL4-3.Luc.R⁺E⁻ viral vector in place of the nef gene, was impervious to the presence of huSlfn11 (FIG. 7B, top), thus starkly contrasting the huSlfn11-mediated diminishment of all other proteins encoded on this vector. We therefore decided to test the effect of huSlfn11 on the expression of Nef in the context of pNL4-3-DEnv-EGFP which has part of the env gene replaced by EGFP, but harbors nef in its original position. Strikingly, as all other viral proteins Nef expression was inhibited by huSlfn11 (FIG. 7B, bottom). In summary, these findings lead us to conclude that huSlfn11 selectively suppresses viral protein expression via transcript-intrinsic properties, rather than through external regulatory factors or positional elements. This notion was further corroborated by the fact that huSlfn11 had no effect on an EGFP-RRE reporter system designed to monitor RRE-mediated events such as nuclear export of unspliced RNA.

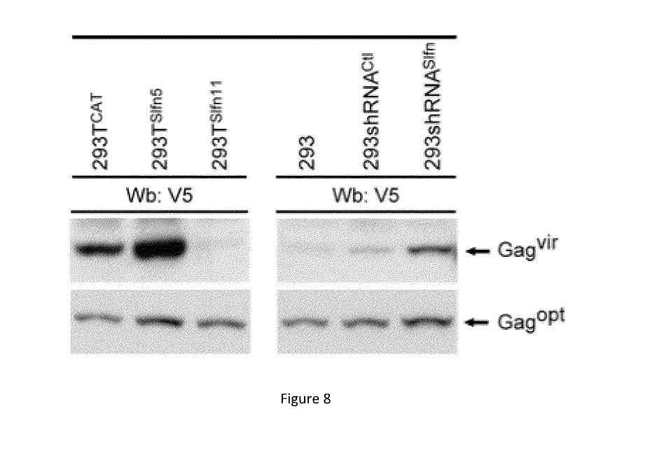

Viral genomes are known to display biased nucleotide compositions that are quite different from human genes (30-39). For instance, extremely high frequencies of A nucleotides are found in the RNA genomes of the lentivirus group of retroviruses and of influenza virus (33, 37, 40), (41). The sequences of wild-isolate strains of HIV-1 are characterized by low GC content and suboptimal codon usage when compared to the host cell bias which is particularly marked for the gag and pol genes (34, 42-45). The unusually high AU content imparts a rare codon bias favoring A/U in the third position, and their presence has been demonstrated to induce ribosome pausing and inefficient translation. As the inhibitory effect of huSlfn11 on viral protein expression was found intrinsic to the affected transcript, we contemplated that huSlfn11 exploits the viral codon preference to specifically attenuate viral protein synthesis. To this end, we generated mammalian expression vectors containing only the ORF of HIV-1 gag with either the original viral codon bias as present in HIV-1 (Gag^{vir}), or with synonymous codon usage optimized for expression in human cells (Gag^{opt}). As is clearly evident in FIG. 8, huSlfn11 strongly affected the expression of Gag^{vir}, but was without any significant consequence for the expression of Gag^{opt}.

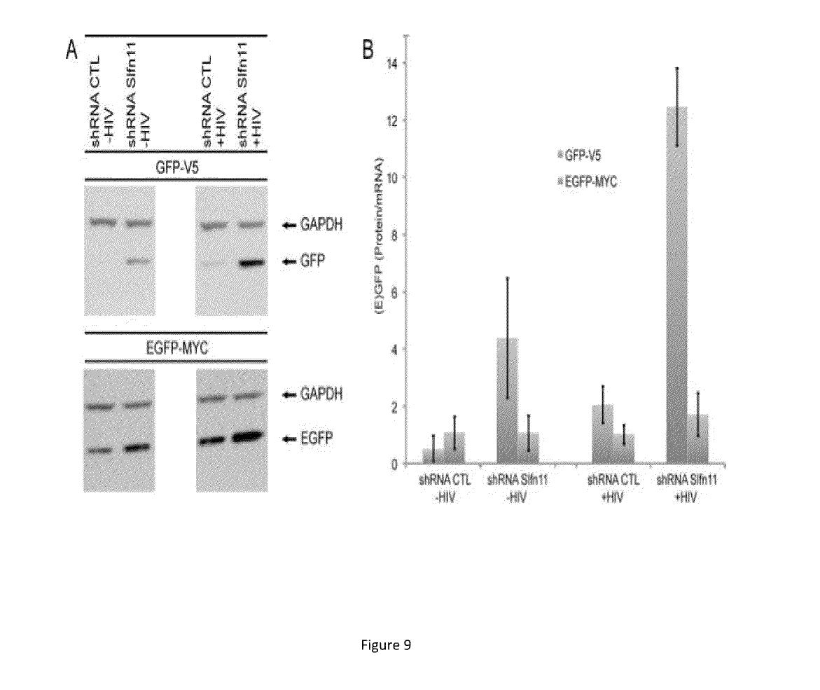

To unequivocally demonstrate that truly codon bias, and not the loss of other functions found in the Gag sequence (e.g. Rev binding- or cryptic splice sites) accounted for the observed differences, we also employed two GFP constructs (pcDNA5.1) that contain either A/T-biased codons (GFP) or G/C-biased codons (EGFP), but with identical protein sequence. As seen in FIG. 9, significant modulation of expression by huSlfn11 only occurs in the case of GFP, whereas translation of EGFP was only slightly affected by huSlfn11 (A/T biased codons did not significantly alter mRNA levels, and the minor differences have been corrected for in FIG. 9B). Importantly, the difference between the two GFPs increased in the presence of proteins derived from pNL4-3.Luc.R⁺E⁻, consistent with the reported altered tRNA pool during HIV infection (46). From these finding, without limiting the invention to any particular mechanism, the inventors are of the view that huSlfn11 is exploiting the distinct viral codon bias to selectively attenuate the expression of virally encoded proteins (note: increased susceptibility to APOBEC3G or methylation of integrated viral DNA might account for the lack of adaption of the viruses to utilize G/C-rich codons).

In summary, our systematic analysis of the individual stages of the HIV replication cycle revealed that huSlfn11 does not inhibit reverse transcription, integration, or production and nuclear export of viral RNAs. Furthermore, we did not observe a block in the budding or release of viral particles. However, we discovered a selective inhibition in the synthesis of virally encoded proteins. Without limiting the invention to any particular mechanism, the inventors are of the view that huSlfn11 interferes at the point of virus protein synthesis by exploiting the unique viral codon bias towards A and T nucleotides in the third position. Surprisingly, the A/T bias is significantly less prominent in the early viral gene products such as Rev or Tat, which is consistent with our observations that the generation of viral RNA and its export in unspliced forms appears impervious to huSlfn11. This model is also consistent with our observation that the antiviral activity of huSlfn11 extends to other retroviruses (and possibly other viruses with rare codon bias such as influenza). Our results clearly establish huSlfn11 as a potent, interferon-inducible restriction factor against retroviruses such as HIV, and future studies will determine whether other members of the Slfn family possess antiviral activity against distinct groups of viruses. To our knowledge this is the first evidence for a restriction factor to mediate its antiviral effects on the basis of synonymous codon bias.

Our lab has extensive experience in the characterization of the molecular mechanisms that govern the biological responses to interferons and pathogens, and in the analysis of signaling pathways and nucleic acid binding proteins. We are working with two local virologists recognized for their work on HIV and other viruses, Dr. Young and Dr. Guatelli, to elucidate the anti-(retro)viral effects of Slfns. In addition, we will characterize the effects of huSlfn11 on the tRNA pool.

Our salient findings are:

1) Both human and murine Slfn proteins are inducible by IFNα/β, or TLR ligands such as dsRNA or LPS

2) Slfn proteins contain a closely conserved amino-terminal domain albeit of no sequence similarity to any other protein outside the family. Limited homology to a deviant AAA+ domain found in bacterial RNA/DNA helicases is contained in a 100 aa stretch within this NH2-terminal region

3) In over a dozen human cell lines or primary cells tested, huSlfn11 was the prevailing family member

4) HEK293 cells express several Slfn proteins, whereas HEK293T cells lack almost all Slfn expression

5) When transfected with MSCV-IRES-GFP and pCL-Eco helper constructs, or with pNL43LucR+E−HIV and pCMV-VSVg Env plasmids, HEK293T cells produce substantially more virus then HEK293 cells

6) Ectopic expression of huSlfn11 (but not huSlfn5) in HEK293T cells dramatically inhibits retrovirus production, whereas ablation of Slfn11 expression in HEK293 cells significantly boosts virus production

7) The expression of huSlfn11 does not cause a ‘generic’ antiviral state, as replication and production of DNA viruses (AAV and HSV) is not affected by huSlfn11 expression. Furthermore, no modulation of ISG induction by IFNα/β or TLR ligands was observed as a consequence of huSlfn11 expression.

8) huSlfn11 expression does not affect the early stages of retroviral replication when cells were infected with VSVg-pseudotyped HIV (as entry is bypassed with VSVg-pseudotyped HIV, these steps encompass reverse transcription, integration, and transcription from the integrated sequence). Consequently, no effect of huSlfn11 on intracellular vRNA levels, splicing or export was observed.

9) In contrast, attenuated vRNA and p24 levels in the supernatants of huSlfn11-expressing corroborated our results obtained from the viral titer determination via infection assays. The inhibitory effect of huSlfn11 is independent of the presence of viral envelope protein (production of non-enveloped viral capsids is inhibited by huSlfn11)

10) EM analysis was unable to identify a specific block at the budding or release stage of virus production

11) huSlfn11 potently inhibits synthesis of viral, but not cellular proteins in both uninfected and more prominently in HIV-infected cells, based on differences in codon bias between virus and host cell

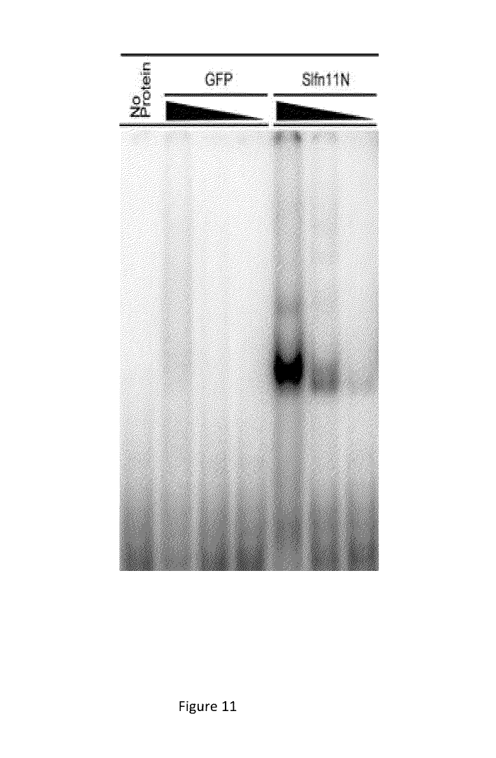

12) huSlfn11 can bind directly to tRNA in EMSAs and alters the tRNA pool of the host cell (FIGS. 11 & 12)

13) Viral replication studies using WT HIVLAI and the CD4+ T cell line CEM confirmed the anti-retroviral function of huSlfn11

### Example 2

### Aim 1. To Define the Functional Domains in Slfn11

1.1 Investigate the Role of the AAA+ Domain in the Function of Slfn11—

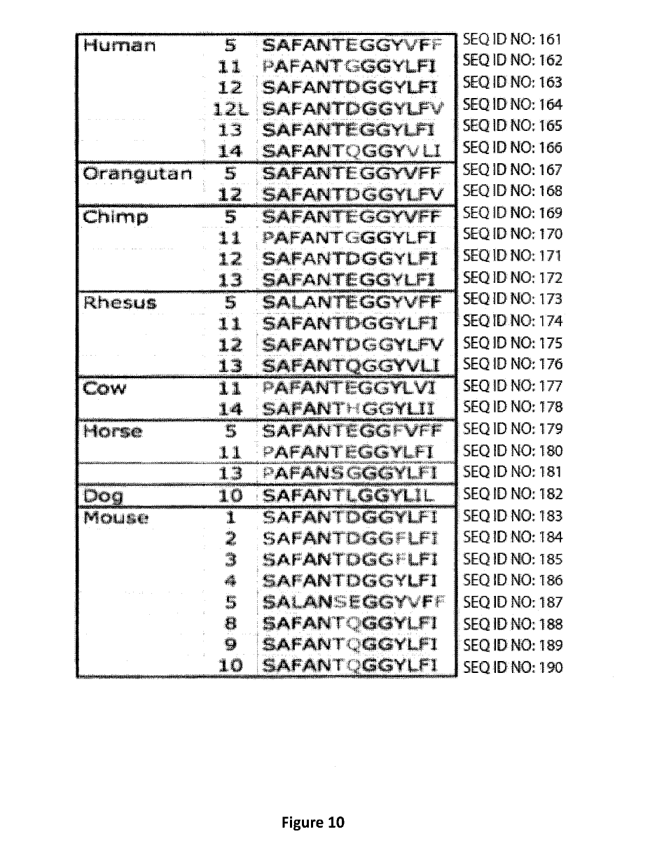

The ATP-binding AAA+ domain is the only well conserved motif found within all Slfn proteins, and a comprehensive alignment of all Slfn AAA+ domains with AAA sequences from other proteins revealed a number of highly invariant residues (e.g. the “FANT” or “GG” sequences), suggesting they contribute to the function of these proteins. Notably, all Slfn11 proteins carry a proline instead of a serine residue in the first position of the shown aligned AAA+ motifs (see FIG. 10). In order to determine the role of the AAA+ motif in huSlfn11 function, we propose to mutate the invariable “GG” sequence in huSlfn11, as well as to replace the first-positioned proline with a serine residue. In addition, we will create mutations in the AAA+ domain that have been determined in other AAA+-containing proteins to affect function. We will then determine the ability of these mutants to inhibit retrovirus production, and in parallel analyze their ability to bind and/or hydrolyze ATP in-vitro. The latter will be performed by incubation of the purified huSlfn11 protein with either ³²P-ATP, or the non-hydrolyzable ATP-analog ³²P-AMP-PNP. We will also mutate the AAA+ motif in huSlfn5 to the sequence present in huSlfn11 to determine whether the inability of huSlfn5 to inhibit retrovirus production lies within its different AAA+ domain. We predict that some of these AAA+ mutants will be defective in either ATP-hydrolysis and/or ATP-binding. The determination of the biological activity of such mutants will allow us to determine whether ATP-hydrolysis is required huSlfn11 to attenuate retrovirus production (which would indicate an as of yet unidentified enzymatic function of huSLfn11), or whether ATP-binding ‘merely’ promotes a conformational change in the protein that allows it to oligomerize and to interact with nucleic acids as has been reported for other AAA+ proteins (summarized in (47); see Aim 1.2).

1.2 Characterize Whether huSlfn11 Acts in Oligomeric Form—

Oligomeric assembly is a hallmark of many AAA+ proteins and is a prerequisite for their biological function (47). It is therefore important to determine whether huSlfn11 can form oligomeric structures, and whether such larger complexes form as a consequence of ongoing virus production. We will use two complementary approaches to determine the formation of multimeric complexes. First, we will co-transfect plasmids encoding huSlfn11 with several distinct epitope tags (e.g. HA-huSlfn11, FLAG-huSlfn11, etc). Immunoprecipitation with antibodies directed against one tag, followed by Western blot analysis for the presence of the other tags will allow us to determine the formation of oligomers. Additionally, we will subject lysates from huSlfn11 expressing cells to size exclusion chromatography followed by immunoblot analysis of the resulting fractions. The first approach provides a straightforward system for the identification of the domains involved in oligomerization, whereas the second approach allows for the determination of the extent of oligomerization (e.g. pentamer, hexamer, etc.). Both approaches will be pursued in the presence and absence of virus production to resolve whether the presence of viral RNA or particles presents a trigger for the assembly process. Considering that most AAA+ proteins function as oligomers, we hypothesize that the same is true for Slfn proteins. In this case we will continue by mapping the Slfn ‘oligomerization domain’ with the ultimate goal of generating assembly-deficient mutants to clarify the role of oligomerization in Slfn function. The proposed experiments involve standard techniques, so it is not likely that we will encounter any significant technical challenges in the analysis, and the results will undeniably explicate if huSlfn11 oligomerization occurs, and if yes, whether it is of vital consequence for huSlfn11 function. In conjunction with the other parts of Aim 1, this subaim will provide critical insights into structural elements and the molecular mechanism that governs huSlfn11's function as a retroviral restriction factor. Additional significance of the SNP analysis proposed in Aim 3.2c will emerge when these results are put into the context of the collective findings obtained in Aims 1.1-3.

1.3 Define the Nucleic Acid Binding Domain(s) of huSlfn11—

Our preliminary evidence demonstrated that huSlfn11N can bind to nucleic acids such as tRNA (see FIG. 10). Strikingly, no discernible (nucleic acid binding) domains of any kind could be identified within this short Slfn region, with the exception of the putative AAA+ motif. The NH2-terminal region of all Slfns is highly conserved (see FIG. 1), and our functional analysis of huSlfn11 revealed that this NH2-terminal region of huSlfn11 is equipotent to full-length huSlfn11 in the inhibition of retrovirus production (see FIG. 3). FIG. 1 further illustrates that long Slfn isoforms (including huSlfn11) contain an additional candidate nucleic acid binding domain, the DEAQ/Walker motif. The collective of our preliminary studies has lead us to our working hypothesis that huSlfn11 interferes with viral protein synthesis, most likely through interaction with tRNA (see Aim 2). One conserved feature of tRNAs is the presence of three stem-loop structures found in all tRNAs. Using partial tRNA sequences we will define the Slfn-binding region and test whether a specific stem loop is recognized by huSlfn11 binding (note: results from aim 2 will influence which tRNA(s) will be used as probes in these assays). In order to define the RNA-binding domain of huSlfn11, we will generate NH2- and COOH-terminal as well as internal deletion and point mutants of huSlfn11 to map the regions required for nucleic acid binding. In addition, we will introduce mutations into huSlfn11 known to disrupt nucleic acid interaction within other DEAQ-containing proteins. These experiments will not only provide us with vital information on the requirements and the role of huSlfn11 nucleic acid binding in its biological function, but will also allow us to generate huSlfn11 mutants that would predictably act as dominant-negative factors over wild-type huSlfn11 in the inhibition of retrovirus production. We have several assays in place to determine the nucleic acid binding capabilities of the different huSlfn11 proteins in-vitro (e.g. as shown in FIG. 11), and all huSlfn11 mutants will be subject to functional analysis in-vitro and in-vivo. We are also currently generating reporter constructs to facilitate faster initial screening of Slfns and their mutants. Specifically, we are placing the firefly luciferase coding sequence utilizing virus-favored codons and the cDNA for Renilla luciferase with human codon bias into pcDNA 5.1, separated by an IRES sequence. As both proteins are derived from the same mRNA, any changes in their ratios will indicate preferential inhibition of protein synthesis (note: two complementary constructs placing either the firefly or the renilla luciferase before the IRES will preclude any potentially misleading results due to interference with the IRES function).

1.4 Explore Whether the Subcellular Location of huSlfn11 Changes During Retroviral Infection—

Preliminary immunohistochemical analysis using transiently expressed epitope-tagged huSlfn11 showed diffuse cytoplasmic staining of the protein. To corroborate this observation, we will create GFP-huSlfn11 constructs and generate stable HEK293T cell lines to avoid potential obscuring of specific sub-cellular localization through overexpression of the fusion protein. As we do not know at this point whether GFP fusion will affect the function of huSlfn11, we will create both NH2- and COOH-terminal fusion proteins, and test them first for their ability to attenuate retrovirus production in HEK293T cells. We will then examine the intracellular distribution of huSlfn11 in ‘resting’ cells, as well as in cells that have either been infected with replication-deficient VSVg-HIV, or that have been transfected with the pNL43LucR⁺E⁻HIV plasmid to determine whether either early or late stage events, respectively, of the viral infection cycle alter the subcellular localization of huSlfn11. As huSlfn11 expression does not affect the early stages of viral infection including integration and transcription, we predict that VSVg-HIV infection in the absence of new particle formation will not trigger a redistribution of huSlfn11 (e.g. its affiliation with mitochondria similar to the RIG-I helicases). In contrast, we would not be surprised to find that huSlfn11 ‘accumulates’ in the ribosomes. These experiments are technically straightforward, and will provide a definitive answer to the posed question. If we find that huSlfn11 translocates within the cell, we will follow up by investigating the mechanism that controls that movement (e.g. mass-spectrometric analysis to identify possible post-translational modifications of huSlfn11). Without limiting the invention to any particular mechanism, the inventors are of the view that if no movement of huSlfn11 is observed even though inhibition of virus production occurs, then this shows that accumulation at specific subcellular compartment is not required for the anti-retroviral function of huSlfn11.

### Example 3

### Aim 2. To Characterize the Inhibitory Effect of Slfn11 on Viral Protein Synthesis

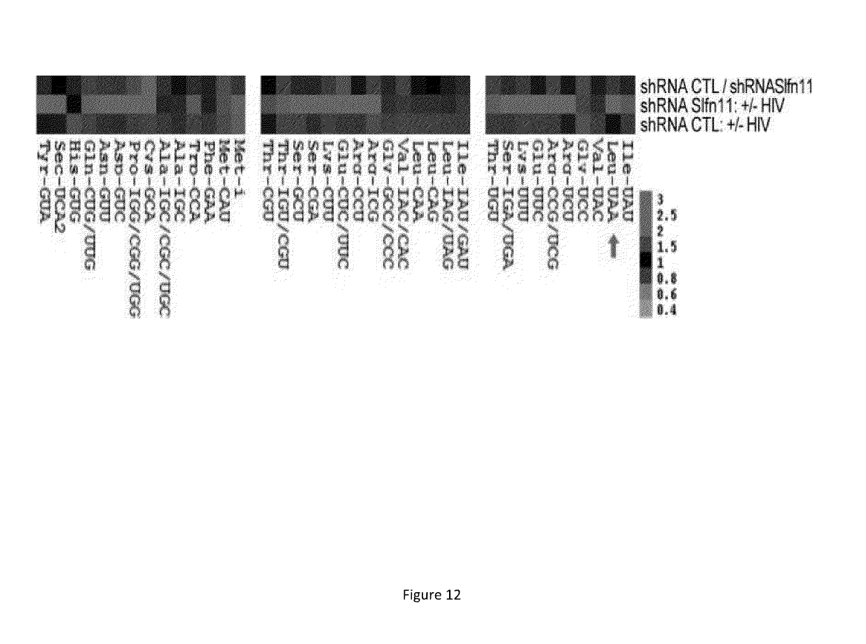

Our collective preliminary findings already allow us to eliminate a large number of steps in the retroviral reproduction cycle as targets of huSlfn11 action, and strongly support the notion that huSlfn11 inhibits retrovirus production by attenuating viral protein synthesis through interference with tRNA function. Initial experiments revealed a dramatic change in the tRNA pool during HIV infection in huSlfn11-deficient cells (FIG. 12, middle), but not in cells expressing huSlfn11 (bottom). Consistent with our findings shown in FIG. 9, the effect of huSlfn11 becomes significantly more prominent in the presence of viral proteins. Particularly striking is the strong alteration of the tRNA for the UAA codon (red arrow), which accounts for 42% of Leu in HIV-Gag, but only 7% of Leu in the human genome.

In order to characterize the mechanism by which huSlfn11 attenuates retroviral protein synthesis production, we propose to:

2.1 Investigate Whether huSlfn11 Preferentially Targets Specific tRNAs—

EMSAs using total tRNA as probe revealed clear dose-dependent shifting (FIG. 11), and at the highest amounts of huSlfn11 all of the probe was shifted, indicating that at least in-vitro, huSlfn11 is able to binds all tRNAs (this notion is also supported by the above shown tRNA array, however, we can at this point not exclude the possibility that HIV- or huSlfn11-mediated changes in specific tRNAs trigger a change in the total tRNA pool). To determine whether huSlfn11 binds to certain tRNAs with higher affinity, we will perform EMSAs with an amount of huSlfn11 that results in partial shifting, excise both the shifted and unshifted bands, and employ the tRNA arrays to analyze the tRNA composition in each band. If huSlfn11 binds distinct tRNAs with higher affinity, we will see an ‘enrichment’ of those tRNAs in the shifted band. In this case, without limiting the invention to any particular mechanism, the inventors are of the view that tRNAs complementary to codons favored by the virus would be the ones displaying greater affinity to huSlfn11, and the array results would certainly reveal the identify of those ‘preferred’ tRNAs. If the tRNA distribution is the same in both bands, we have to conclude that huSlfn11 binds all tRNAs indiscriminately. In that case, the logical conclusion would be that the impact of huSlfn11 on less abundant tRNAs (corresponding to viral codon bias) would be significantly greater than on highly expressed tRNAs.

2.2 Investigate how huSlfn11 Alters the Cellular Pool of tRNAs—

The striking modulation of the cellular tRNA pool by the presence of HIV proteins (FIGS. 12 and (41)) is consistent with the fact that HIV-Gag encoded protein(s) interact(s) with numerous tRNA-synthetases (Source: HIV-1 Human Protein Interaction Database, NCBI). We are particularly interested in MA, as this small protein interacts with the tRNA synthetases in genome-wide association studies (N. Krogan; personal communication). However, it remains undetermined whether tRNA itself participates in this interaction, and we will therefore begin by testing whether Gag/tRNA-synthetase interaction is sensitive to RNAse treatment. We will then determine which of the HIV-derived protein(s) account(s) for the virus-induced changes in tRNA expression seen in FIG. 12 (the experiment shown employed the pNL4-3.Luc.R⁺E⁻ vector, thus, most viral proteins were expressed). We have (codon-optimized) expression constructs for all viral proteins, including the individual Gag-derived proteins. Based on the published interaction studies, Gag, or MA specifically, are our prime candidates.

The experiment depicted in FIG. 12 reflects the total tRNA concentration (aminoacylated=charged and uncharged tRNAs), however, a modification of sample preparation for the array experiments will allow for the distinction between charged and uncharged tRNAs (1). One possibility how huSlfn11 might counteract the HIV-mediated changes in tRNAs is by preventing charging of tRNAs. Recognition of individual tRNAs by their respective tRNA synthetases is crucially dependent on the distinct structures of the tRNAs. A bioinformatic alignment of the AAA+ domain of huSlfn11 revealed that it is most closely related to AAA+ domains found in bacterial RNA/DNA helicases. It is therefore possible that huSlfn11 possesses helicase activity, and that such activity could be directed against tRNA stem-loop structures, thus preventing charging of the tRNAs. Alternatively, the Gag/tRNA-synthetase complex and huSlfn11 might bind competitively to the tRNA. Regardless, the lack of aminoacylation of tRNA has been linked to reduced tRNA half-life (48), thus, inhibition of tRNA charging could lead to a decline in total tRNA concentration. We will therefore test 1) whether expression of huSlfn11 affects tRNA charging in vivo in the presence and absence of viral infection, and 2) whether purified, recombinant huSlfn11 interferes in in-vitro tRNA charging assays (note: results from aim 2.1 will determine which tRNA/tRNA synthetase will best be used as substrate). Although less likely, we also consider the possibility that huSlfn11 interferes with the addition of the CCA-tail by the nucleotidyltransferase during tRNA maturation, since this, too, could account for the changes in tRNA levels.

The function of Slfn proteins had remained a mystery for over a decade, and we have made great progress in relatively short time not only in identifying huSlfn11 as a retroviral restriction factor, but also in substantially narrowing down its point of action in the viral life cycle. While clearly still several possibilities exist with regard to the exact molecular mechanism by which huSlfn11 selectively alters translation efficiency, we are confident that, through a systematic step-by-step process, we will be successful in characterizing the process by which huSlfn11 inhibits the production of viral proteins.

### Example 4

### Aim 3. To Determine the Specificity of Slfns for Inhibition on HIV Replication

3.1 a) Test the Additional Human Slfn Proteins for their Ability to Alter HIV Replication—

Our results thus far have demonstrated a certain level of specificity of Slfn function, as huSlfn11 did not alter adenovirus replication, nor did huSlfn5 affect retrovirus production. We are currently cloning the remaining huSlfn family members, and will test their ability to attenuate retrovirus production using the same two retroviral packaging/production systems (MSCV-IRES-GFP & pCL-Eco; pNL43LucR⁺E⁻HIV & pCMV-VSV-g Env) we employed in our preliminary studies. We will use HEK293T cells for these studies as we were unable to detect any endogenous Slfn expression in these cells. We do not anticipate any technical problems with this subaim, as we have already performed these experiments with huSlfn11.

3.1 b) Confirm the Effects of huSlfn11 on WT-HIV Replication in T Cells—

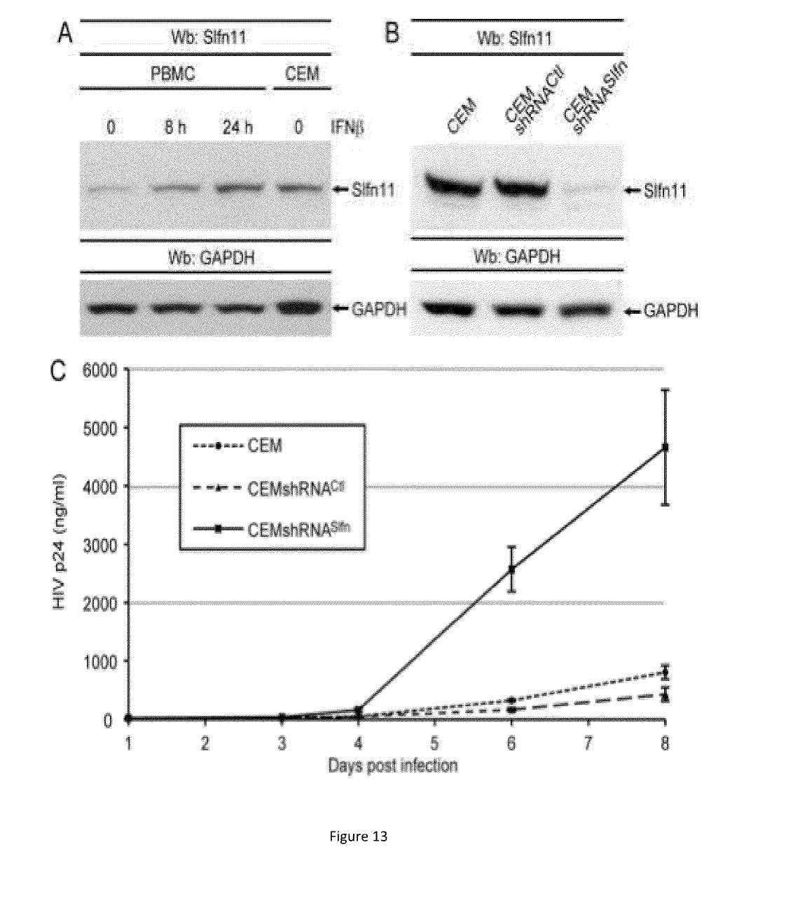

Our data illustrate that Slfn11 inhibition of virus production is not unique to HIV, but also extends to Moloney Murine Leukemia Virus-based MSCV, suggesting a broader anti-retroviral mechanism. We still need to further corroborate our findings on HIV production with WT rather than VSVg-pseudotyped HIV, and therefore need to use a huSlfn11-deficient CD4⁺ T cell line. Our HIV/AIDS core facility (which performs these experiments on a recharge basis) routinely uses CEM cells to quantitate HIV replication. We have determined that huSlfn11 expression in CEM cells is approximately equal to that in human PBMCs, and we have created huSlfn11-deficient CEM cells using the same shRNA construct we used to knock down huSlfn11 in HEK293 cells. As shown in FIG. 13, a pilot replications kinetic study of WT HIV_{LAI} in CEM, CEM-Slfn11^{shRNA} and CEM-Ctl^{shRNA} cells lends further support to a potent antiviral activity of huSlfn11. We will confirm our findings in 293 cells relating to the effects of huSlfn11 to intracellular HIV protein and vRNA levels in these CEM cells. We will also test different HIV-1 strains as well as HIV-2 to determine whether different susceptibility to huSlfn11 exists between these viruses.

3.1 c) Investigate Whether a Functional Murine Homolog to huSlfn11 Exists—

Retroviruses are not unique to primates, and it is therefore likely that a functional homolog to huSlfn11 exists in the mouse. Five long muSlfns exist with approximately equal sequence similarity to huSlfn11 (muSlfn 5, 8, 9, 10 & 14) and it is possible that one of these proteins is capable of inhibiting retrovirus production including HIV. We have already generated plasmids encoding muSlfn2, 3, 8, 9 and 10, and are in the process of cloning the remaining family members. If we find an anti-retroviral muSlfn, it will serve as the basis for the future generation of knock-out mice (while this would be certainly our next goal, we believe that in the absence of further evidence it is beyond the scope of this application). If we find that none of the muSlfns exhibit anti-retroviral activity, it will provide us with important information regarding the functional domains of Slfn proteins, as murine and human Slfns are well conserved. It is also intriguing to theorize that HIV has evolved countermeasures against huSlfn11 (aim 3.2), which might be ineffective against a murine Slfn11 homolog (species-specific effects of host cells proteins have already been demonstrated, e.g for TRIM5α (49)). As such, an unopposed murine Slfn family member could contribute to the block of HIV replication in murine cells, and a better understanding of this process will bring the field one step closer to a mouse model of HIV infection.

3.2 a) Determine Whether Virus-Encoded Slfn Proteins Act Dominant-Negative Over huSlfn11—

The only non-mammalian ‘organisms’ that encode slfn-like proteins are all members of the Pox-viruses including among others cowpox, camelpox, mousepox etc. (50, 51). As illustrated in FIG. 1, the viral Slfns resemble in sequence and length most closely the short murine Slfns 1 and 2, which are absent in humans. Interestingly, bioinformatic analysis revealed a fragmented structure of the vSlfn genes of the human-tropic variola and vaccinia virus strains, precluding the expression of a ‘full-length’ vSlfn, but not necessarily excluding the possibility of the expression of shorter proteins. In contrast, the genomic sequence of the poxvirus Molluscum contagiosum virus (MCV), a very common cause of viral skin and mucous membrane infection, does not disqualify the virus from expressing a vSlfn protein. As vSlfn genes are present in all poxvirus genomes regardless of species specificity, it is likely that these proteins benefit the virus at some level. Even though vSlfns are not found in retroviruses, we are interested in determining their potential function as dominant-negative mutants over huSlfn11, as this might be of relevance in the context of opportunistic co-infection. We will therefore test whether vSlfn from MCV, or predicted truncated vSlfns of vaccinia and variola viruses, can attenuate the anti-retroviral properties of huSlfn11. This will be done in transient expression systems using replication-deficient retroviruses to avoid biosafety concerns. These studies will not only provide valuable insights into the role of vSlfn proteins in potentially promoting retroviral infection, but the sequence disparities between human and viral Slfns will also identify elements in Slfn proteins that are crucial to their function.

3.2 b) Assess Whether HIV-Encoded Proteins can Counteract the Anti-Retroviral Effect of Slfn11—

The innate antiviral mechanisms that are working against HIV, a relatively new human infection, evolved to select against ancient retroviral infection (52). In opposition, an array of HIV “accessory” genes (vif, vpu, vpr, and nef) that serve to evade or subvert the various forms of innate immunity evolved. For example, HIV Vif effectively antagonizes the antiviral effects of the APOBEC3 family of editing enzymes, whereas HIV Vpu counteracts another interferon-induced restriction factor, BST2, to facilitate HIV release from the cell membrane (25). By interfering with the host cell mechanisms involved in intracellular signaling, HIV is able to establish an intracellular niche and persist. In our studies we observed that the inhibitory effect of huSlfn11 on virus production after MSCV-IRES-GFP & pCL-Eco vector cotransfection is consistently more pronounced than when pNL43LucR⁺E⁻HIV & pCMV-VSVg Env plasmids are introduced to generate HIV particles (see FIGS. 3 & 4). The latter plasmids encode all HIV proteins except env and nef, and it is possible that a viral protein counteracts the antiviral effects of huSlfn11. A pilot experiment of cotransfection of pNL43LucR⁺E⁻HIV together with the MSCV-IRES-GFP & pCL-Eco vectors yielded increased MSCV titers, suggesting that the weaker antiviral effect of huSlfn11 on HIV compared to MSCV production is not intrinsic to the VSVg-HIV system, but can be caused ‘in trans’ by the presence of pNL43LucR⁺E⁻HIV. We hypothesize that if HIV indeed directly counteracts Slfn11, then it is likely to do so through one of the other non-structural proteins that are well-recognized virulence factors (Vpr, Vif, or Vpu). We will test their ability to attenuate huSlfn11 function. If we identify a viral huSlfn11 antagonist among these proteins, we will continue by determining its mode of action (e.g. degradation or re-sequestration of huSlfn11). In the case of a negative outcome, we will systematically delete individual coding regions from the pNL43LucR⁺E⁻HIV vector. Cotransfection of these deletion mutants with the MSCV-IRES-GFP & pCL-Eco vector system and subsequent analysis of MSCV titers will allow us to pinpoint the element(s) of HIV that antagonize huSlfn11 function (note: as we do not intend to measure HIV production, we do not need to be concerned about possible detrimental effects of such deletions on HIV particle formation).

3.2 c) Explore Whether Reported SNPs of Slfn11 Affect its Efficacy as a Retroviral Restriction Factor—

As an ancient antiviral defense mechanism, the innate immune response is a collection of functionally distinct subsystems that have evolved to counter infection by viruses. Studies of mechanisms by which HIV evades the antiviral innate immune responses provide the scientific impetus to characterize the genetic polymorphisms that can affect components of the host signaling pathways. Compelling evidence for a genetic basis for the variation observed in human populations in HIV/AIDS susceptibility has come from the analysis of the highly protective CCR5 Δ32 allele, various haplotypes of the promoter for CC chemokine receptors, selected human leukocyte antigen (HLA) alleles, and specific interactions between killer cell immunoglobulin-like receptors (KIR) and HLA class I ligands in HIV/AIDS susceptibility. Thus, natural variations in the expression levels or sequences of host restriction factors that have intrinsic antiviral ability such as huSlfn11 can impact HIV replication. The NCBI SNP database reports 241 SNPs in huSlfn11, and we hypothesize that some of these sequence divergences may have emerged due to altered efficacy of these huSlfn11 variants against retrovirus/HIV production. With the assistance of the UCSD HIV/AIDS Bioinformatics and Genomics core we will analyze whether these SNPs are localized in ‘hot-spots’, or are found in sequences that we identify as critical functional domains in Aim 1. In that case we will introduce these “mutations” into the huSlfn11 expression vector and test the antiviral ability of the mutants. Cluster analysis and the results from Aim 1 will allow us to limit the number of mutants to be tested. We do not anticipate any technical problems with these experiments, as we routinely produce point mutants on a large scale (e.g. we just generated a series of 55 K->R mutants of STAT1). While this subaim represents a small scale screening effort rather than a specifically targeted experiment, we believe that the data obtainable from this subaim will provide a foundation for future studies using primary patient samples and clinical data to determine a prospective role of huSlfn11 SNPs in disease progression. Identification of SNPs affecting huSlfn11's function as an HIV restriction factor will not only provide a better understanding of the functional elements of huSlfn11, but also carries potential as a diagnostic determinant of HIV/AIDS susceptibility.

Most of the studies outlined in this application will be using HEK293 cell lines, as we already extensively characterized the antiviral mechanism of huSlfn11 in this cell system and it clearly provides numerous advantages for the proposed mechanistic experiments (e.g. availability of large numbers of easily transfectable cells, and the existence of complimentary “matched” cell lines). Naturally, we intend to verify and confirm all seminal findings in the CEM cell-based experimental setup as described in FIG. 13, and whenever feasible in primary CD4⁺ lymphocytes isolated from PBMCs.

### Example 5

### Methods Used in Examples 6-15

Plasmids and Antibodies.

huSlfn5 and huSlfn11 were cloned into pcDNA6/V5-His. PCR fragments of huSlfn11 were cloned into pcDNA6/V5-His to generate huSlfn11N (aa 1-579) and huSlfn11C (aa 523-901). The pNL4-3.Luc.R⁺E⁻ HIV-1 vector has been described previously²⁸. huSlfn11 antibodies were from Sigma and Abmart. pNL4-3-DEnv-EGFP, pNL-GFP-RRE(SA) and antibodies against HIV-1 proteins were obtained through the NIH AIDS Research and Reference Reagent Program. Generation of Gag^{vir}, Gag^{opt} and (E)GFP vectors is in Methods. pLKO.1 shRNA lentivirus constructs were from Open Biosystems.

Virus Production and Titer Assays.

HIV production was determined by transfection of pNL4-3.Luc.R⁺E⁻ and pCMV-VSV-G into cells of interest. Supernatants were used to spin-infect HEK293T cells, and luciferase was measured by Bright-Glo Luciferase Assay (Promega). p24 ELISAs were performed at the Center for AIDS Research, UCSD using Alliance p24 ELISA kits (Perkin Elmer).

HIV Replication Kinetics.

Wildtype X4 strain of HIV-1_{LAI} was used to inoculate CEM, CEMshRNA^{Ctl} and CEMshRNA^{Slfn} cells at an MOI of 0.01, and viral titers were measured by p24 ELISA.

Cell Culture.

HEK293, HEK293T, NIH3T3, Hela, HFF (Human Foreskin Fibroblasts) and their derivative cells were maintained in high glucose DMEM. CEM and their derivative cells were maintained in RPMI 1640 medium. Both media were supplemented with 10% heat-inactivated fetal bovine serum, 100 U/ml Penicillin, 100 μg/ml Streptomycin, 2 mM L-Glutamine, MEM Non-essential Amino Acid, 1 mM Sodium Pyruvate and 55 μM 2-Mercaptoethanol. The Phoenix amphotropic retrovirus packaging cell line (293T) originates from the lab of Dr. G. Nolan (Stanford University, Stanford, Calif.). Human PBMCs were obtained from the San Diego Blood Bank.

Plasmids.

The template vectors encoding huSlfn5 and huSlfn11 sequences were acquired from Open Biosystems (Human MGC Verified FL cDNA (IRAU), clone ID 40123369 and 6258140). The coding sequences were amplified by PCR with PfuUltra (Stratagene) and subcloned into the pcDNA6/V5-His vector (Invitrogen) for mammalian cell expression. Partials of the hSlfn11 coding sequence were amplified by PCR and subcloned into the pcDNA6/V5-His vector to generate the huSlfn11N (1-579) and hSlfn11C (523-901) truncation mutants. The pcDNA6/V5-His CAT (Chloramphenicol Acetyl Transferase) vector was generated the same way using pcDNA5/FRT/TO CAT (Invitrogen) as the PCR template for the CAT sequence. The following plasmids were obtained from the addgene plasmid repository (Cambridge, Mass.): MSCV-IRES-GFP (Addgene plasmid 20672: source David Baltimore); pCL-Eco retrovirus packaging vector (Addgene plasmid 12371: source Inder Verma); psPAX2 lentivirus packaging vector (Addgene plasmid 12260: source Didier Trono); pMD2.G helper vector (Addgene plasmid 12259: source Didier Trono) and pCMV-VSV-G vector (Addgene plasmid 8454: source Robert M. Weinberg). The following plasmids were obtained through the NIH AIDS Research and Reference Reagent Program, Division of AIDS, NIAID, NIH: pNL-GFP-RRE(SA)(Cat#11466) from Dr. John Marsh and Dr. Yuntao Wu, pNL4-3-deltaE-EGFP (Cat#11100) from Drs. Haili Zhang, Yan Zhou, and Robert Siliciano. The pNL4-3.Luc.R⁺E⁻ HIV-1 vector has been described previously²⁸. The pNL4-3.Luc.R⁺E⁻PRstop construct was created by deletion of Thymidine₂₄₇₈ in pNL4-3.Luc.R⁺E⁻ HIV-1 to put the following ‘TAGTAG’ into frame such that no pol-encoded viral enzyme proteins would be expressed. Gag^{vir} and Gag^{opt} constructs: Gag sequences were cloned into pcDNA6/V5/His between Afl II and Xho I sites. Nine base pairs of the wild-type viral sequence preceding the initiation codon were present in both final constructs to assure identical translation initiation sites. Gag^{vir} was generated through PCR amplification of the gag sequence from pGag-EGFP (NIH AIDS Research & Reference Reagent Program, #11468) in which only the Inhibitory RNA sequence (INS) was eliminated through introduction of silent mutations while otherwise retaining the original viral codon usage. Gag^{opt} was generated through PCR amplification of the gag sequence from p96ZM651gag-opt (NIH AIDS Research & Reference Reagent Program, #8675) in which the entire gag sequence was converted to employ codon usage in line with highly expressed human genes. GFP and EGFP sequences were cloned into pcDNA6.2/V5 using the pcDNA6.2/GW/D-TOPO kit (Invitrogen). The GFP sequence was tagged with a V5 epitope while the EGFP sequence was tagged with a Myc tag (GAG CAG AAG CTG ATC AGC GAG GAG GAC CTG). The GFP sequence used retains most of the original wild type protein sequence. The EGFP sequence contains more than 190 silent base changes following human genes codon-usage preferences. Sequences flanking both GFP and EGFP translation initiation site have been converted to a Kozak consensus translation initiation site (C ACC ATG GTG AGC . . . ) to ensure identical translation initiation efficiency in eukaryotic cells.

shRNA Constructs.

The pLKO.1 shRNA lentivirus constructs (TRCN0000148990, TRCN0000152230, TRCN0000155578, TRCN0000157747, TRCN0000152057, TRCN0000155564) against huSlfn11 were obtained from The RNAi Consortium via Open Biosystems (Huntsville, Ala.). Construct TRCN0000148990 (CCGGGCTCAGAA TTTCCGTACTGAACTCGAGTTCAGTACGGAAATTCTGAGCTTTTTTG) produced maximum huSlfn11 knock-down at the protein level and was thus designated as huSlfn11 shRNA in this study. pLKO.1-Blasticidin construct was created by replacing Puromycin resistance gene of the original pLKO.1 construct with Blasticidin resistance gene utilizing BamHI and KpnI sites. The control shRNA construct in the same pLKO.1 vector system (CCGGTGAAGAACTAACCCGGGACTTCTCGAGAAGTCCCGGGTTAGTTCTTCATTTTT G) was also obtained from The RNAi Consortium via Open Biosystems and does not match any human genes.

Antibodies.

Anti-huSlfn11 antibody for immunoblotting was purchased from Sigma Life Sciences (Atlas Antibodies). Monoclonal anti-huSlfn11 antibody for supershift experiments was custom-generated by Abmart. Murine anti-V5 antibody (E10) antibodies were from eBiosciences and Santa Cruz Biotechnology, respectively. Goat polyclonal anti-luciferase antibody was obtained from Promega, Rabbit anti-GFP (D5.1) and rabbit anti-GAPDH monoclonal antibodies (14C10) were acquired from Cell Signaling Technology. The following antibodies were obtained through the AIDS Research and Reference Reagent Program, Division of AIDS, NIAID, NIH: Monoclonal antibody against HIV-1 Gag/p24 (AG3.0) from Dr. Jonathan Allan; anti-HIV-1 RT monoclonal antibody (8C4) from Dr. Dag E. Helland; anti-HIV-1 Vif monoclonal antibody (#319) from Dr. Michael H. Malim; anti-HIV-1 Vpr (1-50) antibody from Dr. Jeffrey Kopp; rabbit anti-HIV-1_{NL4-3} Vpu antiserum from Dr. Klaus Strebel, and anti-HIV-1 Nef (Ag11) monoclonal antibody from Dr. James Hoxie.

Quantitative PCR and Primers.

Total cellular RNA was extracted with QIAGEN RNeasy Mini Kits, and virion RNA in culture supernatants was extracted with QIAGEN QIAamp Viral RNA Mini Kits. Cytoplasmic RNA was prepared with QIAGEN RNeasy Mini Kits following the manufacturer's protocol. Briefly, freshly harvested cells were gently lysed on ice for 5 min in buffer RLN (50 mM Tris.Cl, pH 8.0, 140 mM NaCl, 1.5 mM MgCl₂, 0.5% (v/v) IGEPAL CA-630, 1000 U/ml RNasin® Plus RNase Inhibitor (Promega) and 1 mM DTT). Intact Cell nuclei were removed by centrifugation at 4° C. for 2 min at 300×g, and cytoplasmic RNA was extracted from the remaining supernatant. RNA was cleaned using Ambion DNA-free Kits and reverse transcribed with Applied Biosystems' High Capacity cDNA Reverse Transcription Kit. qPCR was performed on Applied Biosystems 7000 Sequence Detection System or Applied Biosystems StepOnePlus Real-Time PCR System using Power SYBR Green PCR Master Mix. Relative RNA level were calculated after normalization to 18S rRNA unless specified otherwise. The following primer sequences in Table 2 were used in these assays:

TABLE 2
Primer Sequences

| Target Gene | Forward Primer | Reverse Primer |
|---|---|---|
| huSlfn5 | caagcctgtgtgcattcataa | tctggagtatataccactctgtctgaa |
| huSlfn11 | aaggcctggaacataaaaagg | ggagtatatcgcaaatatcctggt |
| huSlfn12 | ctttgttcaacacgccaaga | atgcagtgtccaagcagaaa |
| huSlfn13 | gagaaaatgatggacgcagat | agactcaaaggcctcagcaa |
| huSlfn12L | gaaagtcagtttctgaggaatttca | ccagctcagcatagtttgtgtc |
| huSlfn14 | ggtggtcatgatgctggata | tgatgaaatcaggcaagagttg |
| hISG54 | tggtggcagaagaggaagat | ccaaggaattcttattgttctcact |
| hTBP | gctggcccatagtgatcttt | cttcacacgccaagaaacagt |
| h18S rRNA | ggatgcgtgcatttatcaga | gttgatagggcagacgttcg |
| GFP | ctggagttgtcccaattcttg | tcaccctctccactgacaga |
| EGFP | cagcagaacacccccatc | tgggtgctcaggtagtggtt |
| Luciferase | aggtcttcccgacgatga | gtctttccgtgctccaaaac |
| HIV p24 | tgcatgggtaaaagtagtagaagaga | tgataatgctgaaaacatgggta |

Generation of Stable huSlfn11 Knock-Down HEK293 and CEM Cell Lines.

HEK293T cells were transfected with the huSlfn11 shRNA or control lentivirus vector and the lentivirus packaging vectors psPAX2 and pMD2.G using Lipofectamine 2000 (Invitrogen) according to the manufacturer's protocol. The cell culture medium was collected and replaced with fresh medium 48, 72 and 96 hours after the transfection. The combined supernatants were cleared by centrifugation at 1,000 g at 4° C. for 15 minutes. To generate stable huSlfn11 knock-down HEK293 cell lines, cleared supernatants were added to pre-plated HEK293 cells in the presence of 8 μg/ml polybrene and centrifuged at 600 g for 90 minutes at room temperature. After 48 hours, infected cells were subject to resistance selection with 2 μg/ml puromycin. The efficiency of huSlfn11 knock-down in the selected cells was assayed by qPCR and immunoblotting. To generate stable huSlfn11 knock-down CEM cell lines, cells were first infected and selected as outlined above. Although the same shRNA construct was used, knock-down in CEM cells was less efficient when compared to HEK293 cells. To improve the huSlfn11 knock-down in CEM cells, puromycin-selected CEM cells were re-infected with the pLKO.1-Blasticidin construct carrying the same shRNA and subject to resistance selection with 15 μg/ml blasticidin and 2 μg/ml puromycin. Such double-selected cells displayed a >90% knock-down of huSlfn11 (see FIG. 17b).

Assay for MSCV, HIV and AAV Production.

To analyze MSCV retrovirus production, MSCV-IRES-GFP (MIG) plasmid and pCL Eco packaging vector were transfected into the cells of interest using Lipofectamine 2000 (Invitrogen) according to the manufacturer's instructions. After 24 hours the cells were moved to 32° C., and supernatants were collected after 24 hours and cleared by centrifugation. Transfection efficiency was determined by analysis of GFP-expression in the cells and was subsequently incorporated into the virus titer calculations. The amount of (infectious) virus particles in the supernatants was determined by both infection assays and qPCR analysis of viral RNA. For infection assays, pre-plated NIH3T3 cells were spin infected with 10-fold serially diluted supernatants in the presence of 8 μg/ml polybrene at 600 g for 90 minutes at room temperature. 48 hours after the spin infection, the NIH3T3 cells were examined for GFP expression by flow cytometry, and the number of GFP⁺ cells was used to calculate relative viral titers. For viral RNA qPCR assays, viral RNA was extracted from the virus supernatant and quantitated as described above with EGFP-specific primers. The production of HIV was tested similarly after pNL4-3.Luc.R⁺E⁻ HIV vector, pCMV-VSV-G vector and pcDNA5 GFP vector were transfected into cells of interest using Lipofectamine 2000. The transfection efficiency was established by flow cytometric determination of the number of GFP cells and used to adjust relative viral titers. Viral titers were determined via infection assays, qPCR of viral RNA and HIV p24 ELISA. The infection assay was similar to the MSCV virus infection assay except HEK293T cells were used for analysis. Expression of luciferase was measured by using the Bright-Glo Luciferase Assay System (Promega) and a microplate luminometer (LUMIstar, BMG-LABTECH) 24 hours after the spin infection. HIV viral RNA was quantitated by qPCR using luciferase- or HIV p24-specific primers. The HIV p24 ELISA was performed by the Viral Pathogenesis Core at the UCSD Center for AIDS Research using Alliance HIV1 p24 ELISA kits (Perkin Elmer). For recombinant AAV production assays, cells were transfected with pXX6 (0.6 μg), pXX2 (0.2 μg) and pACLALuc (0.2 μg) using lipofectamine 2000 (Invitrogen), and harvested 72 h after transfection. rAAVLuc-containing lysates were generated by freeze/thaw cycles followed by centrifugation and were used to infect HEK293T cells. Luciferase activity was quantified 48 h post-transduction using Steady-Glo luciferase substrate reagent (Promega) in 96-well Lumiplates (Greiner Bio-One) in a TopCount NXT scintillaton and luminescence counter (PerkinElmer).

Assays for MSCV and HIV Infection.

Amphotropic MSCV-IRES-GFP (MIG) retrovirus was prepared by transfecting Phoenix amphotropic retrovirus packaging cells with MSCV-IRES-GFP (MIG) vector using Lipofectamine 2000. The cell culture medium was collected and replaced with fresh medium 48, 72 and 96 hours after the transfection. The combined supernatants were cleared by centrifugation at 1,000 g at 4° C. for 15 minutes. Cells to be tested were spin infected with the indicated dilutions in the presence of 8 μg/ml polybrene at 600 g for 90 minutes at room temperature. 48 hours after the spin infection, cells were analyzed for GFP expression by flow cytometry. Infection efficiency was established by both the number of GFP⁺ cells and the level of GFP expression. VSV-G pseudotyped HIV was prepared by transfecting HEK293T cells with pNL4-3.Luc.R⁺E⁻ HIV and pCMV-VSV-G vectors using Lipofectamine 2000. The cell culture medium was collected and cleared as described above. Cells to be tested were spin infected with the indicated dilutions in the presence of 8 μg/ml polybrene at 600 g for 90 minutes at room temperature. 24 hours after the spin infection, luciferase expression was measured to determine the relative infection efficiencies.

Transmission Electron Microscopy.

HEK293T cells were transfected with pNL4-3.Luc.R⁺E⁻ HIV vector together with either pcDNA6/V5-His CAT or pcDNA6/V5-His huSlfn11 vectors using Lipofectamine 2000. 48 hours after the transfection, cells were fixed and processed by the Microscopy Core at the Scripps Research Institute. Images were acquired with a Philips CM100 transmission electron microscope.

Whole Cell Lysis and Western Blotting.

Cells were lysed directly in 1×NuPAGE LDS Sample Buffer (Invitrogen) containing 2.5% 2-mercaptoethanol and heated at 90° C. for 5 min. Samples were homogenized by QIAshredder (Qiagen), total protein resolved by NuPAGE (Invitrogen) and transferred to PVDF membranes followed by Western blotting with the specified antibodies. Corresponding HRP-conjugated secondary antibodies and Amersham ECL Plus Western Blotting Detection Reagent (GE Healthcare) were used to visualize the signals followed by quantification using densitometer and NIH ImageJ64 software. Alternatively, fluorochrome-conjugated secondary antibodies were used and signals were acquired and quantified using Typhoon Trio™ Variable Mode Imager and ImageQuant software.

Wild-Type HIV Virus Replication Assay in CEM Cells.

HIV-1_{LAI} was used to infect the CEM stable cell lines (CEM, CEMshRNA^{Ctl} and CEMshRNA^{Slfn}) at an MOI of 0.01 in 5 ml of culture at 0.5×10⁶ cells/ml using RPMI/poly medium containing 3 μg/ml polybrene, 1% glutamine, 100 U/ml Penicillin, 100 μg/ml Streptomycin and 10% FBS. The cultures were sampled at the indicated time points and the viral titers were measured by HIV p24 ELISA.

tRNA Arrays. mirVana miRNA Isolation Kit (Ambion) was used to prepare total RNA from freshly harvested cells following the total RNA isolation protocol. RNA samples were labelled and the relative abundances of individual tRNAs were measured using microarrays as previously described^{25,26}.

tRNA Electrophoretic Mobility Shift Assay (EMSA).

The partial coding sequence of human Slfn11 (a.a. 1-579; huSlfn11N) and the coding sequence of GFP were cloned into the bacterial expression vector pET101/D-TOPO (Invitrogen) with a 6×His epitope tag. 6×His-tagged huSlfn11N and GFP proteins were expressed in Origami B E. coli cells and purified over Ni-NTA and FPLC columns. The purity of the proteins was >95% as estimated by Coomassie Blue Staining. Total human tRNA was extracted with the mirVana miRNA Isolation Kit (Ambion) following the small RNA isolation protocol, and further purified on a 15% denaturing polyacrylamide TBE-Urea Gel (Invitrogen). To generate EMSA probes, purified human tRNA was deacylated to remove charging amino acid, dephosphorylated and 5′-labeled using ³²P-ATP. Total yeast tRNA was purchased from Invitrogen. To prepare the HIV viral RNA, the sequence corresponding to the gag-pol frame-shifting region (2041-2161, pNL4-3.Luc.R⁺.E⁻ vector) was amplified by PCR with T7 promoter-containing primers and in vitro transcribed using MEGAscript T7 Kit (Ambion). Purified proteins, unlabeled competitor RNA or anti-huSlfn11 antibody (Abmart), and probe were combined as specified and incubated on ice for 30 minutes in EMSA buffer (5% glycerol, 10 mM pH7.4 Tris-HCl, 5 mM MgCl₂, 100 mM KCl, 1% Triton X-100, 1 mM DTT and 1U/μl RNasin Plus RNase Inhibitor). After samples were resolved on a 6% DNA Retardation Gel (Invitrogen), the gel was dried and visualized with Typhoon Trio™ Variable Mode Imager (Storage Phosphor Mode).

Data Analysis and Presentation.

Unless indicated otherwise, results are presented in graphs as avg+/−s.d. of at least three independent transfections or infections. For Western blots, a representative of at least three independent transfections or infections is shown. For the experiments shown in FIGS. 16d and e, GFP and EGFP protein and mRNA levels were quantitated and adjusted to the corresponding GAPDH protein or mRNA concentrations, respectively. Expression levels of the proteins were then normalized to their corresponding standardized mRNA concentrations to compensate for minor variations in mRNA concentrations between the samples.

### Example 6

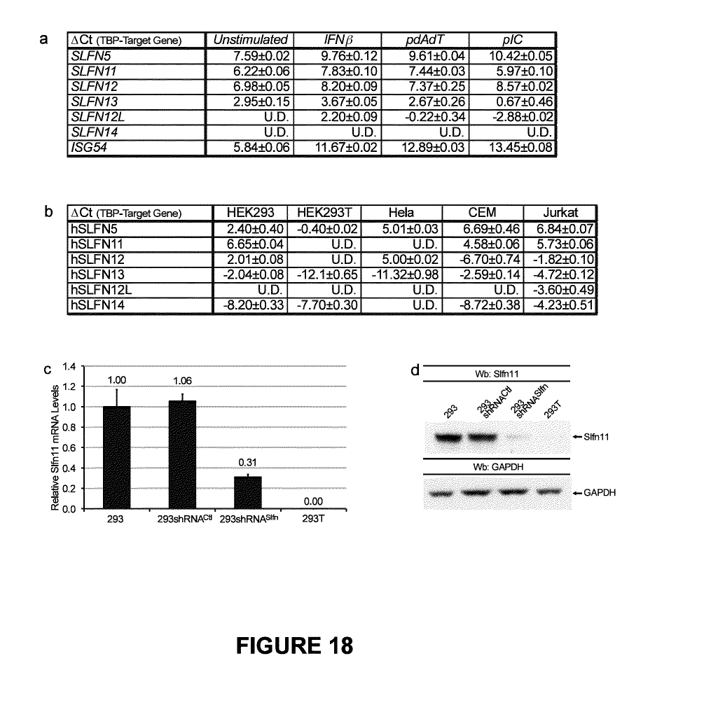

LPS, poly-IC or IFNα/β treatment of macrophages results in induction of several muSlfn genes. Treatment of HFFs with IFNβ, poly-IC or poly-dAdT revealed similar induction of Slfn genes (FIG. 18a), and huSlfn5 and huSlfn11 were consistently the most prominent family members (FIG. 18b). Notably, we observed a striking difference in huSlfn levels between HEK293 (293) and HEK293T (293T) cells (FIG. 18b), and exploited this differential expression to focus on huSlfn11 for further studies. We further employed huSlfn11 shRNA to generate stable 293 cells that specifically lack huSlfn11 expression (293shRNA^{Slfn}) (FIGS. 18c and d).

### Example 7

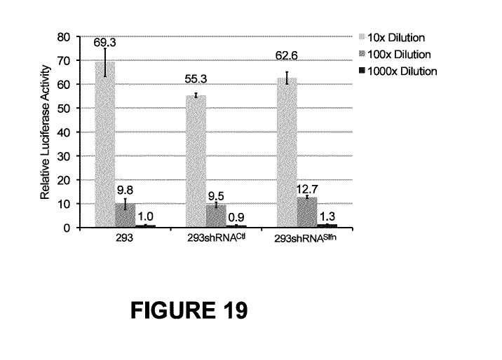

To test whether lack of huSlfn11 in 293shRNA^{Slfn} or 293T cells alters their ability to subdue viral infections, we infected these cells with VSV-G pseudotyped HIV (HIV^{VSV-G}), or amphotropic Murine Stem Cell Virus (MSCV), Adeno-associated Virus (AAV), or Herpes Simplex Virus (HSV). HIV^{VSV-G}-infected cells expressed luciferase after integration of the viral cDNAs into the host genome. Regardless of huSlfn11 expression, all cell lines displayed comparable luciferase levels after HIV^{VSV-G} infection (FIG. 19). A similar lack of influence of huSlfn11 was observed when cells were infected with MSCV, AAV or HSV.

### Example 8

HEK293T cells are used as packaging cells for production of retroviruses, and we therefore considered the possibility that virus production rather than the response towards them is afflicted by huSlfn11. Indeed, 293T cells produced dramatically higher HIV^{VSV-G} (FIG. 14b) or MCSV (FIG. 20a) titers than 293 cells from the viral vectors pNL4-3.Luc.R⁺E⁻ or MSCV-IRES-GFP, respectively. Most importantly, this increase in viral titer was also clearly evident in 293shRNA^{Slfn} cells, whereas 293 and 293shRNA^{Ctl} cells produced the same low levels of virus (FIG. 14b and FIG. 20a). Strikingly, the modulation of virus production is limited to particular viruses, as fabrication of retroviruses (FIG. 14b and FIG. 20a) but not of AAV (FIG. 20c) was affected by huSlfn11. We also did not observe any modulation of ISGs such as ISG15, ISG54 or APOBEC3G as a consequence of huSlfn11 expression, supporting the notion that huSlfn11 does not create a general virus-resistant phenotype.

### Example 9

To corroborate that the observed differences are attributable to dissimilar huSlfn11 expression, we expressed huSlfn11 (aa 1-901) in 293T cells and analyzed their ability to produce HIV^{VSV-G}. Indeed, huSlfn11 radically inhibited HIV^{VSV-G} (FIG. 14a) or MSCV (FIG. 20b) production from 293T cells, with the inhibitory activity residing in the AAA-domain-containing, NH2-terminal region (huSlfn11-N; aa 1-579). No effect of the isolated COOH-terminal region (huSlfn11-C; aa 523-901) harboring the putative helicase sequence was observed (FIG. 14a, FIG. 20b). Surprisingly, huSlfn5 failed to inhibit retrovirus production but yielded slightly elevated viral titers, illustrating specificity among Slfn proteins in their antiviral activity.

### Example 10

To discern whether huSlfn11 reduced the number or the viability of released virus, we measured p24 capsid and viral RNA (vRNA) levels in supernatants of pNL4-3.Luc.R⁺E⁻ transfected, HIV^{VSV-G}-producing cells. Extracellular p24 (FIGS. 14c and d) or vRNA (FIGS. 14e and f) concentration patterns closely matched the titer results from infection assays, demonstrating that huSlfn11 diminishes the number of viral particles released from the cells.

### Example 11

To assess a possible reduction of intracellular vRNA, we determined its levels in 293T cells expressing CAT, huSlfn5, huSlfn11, huSlfn11-N or huSlfn11-C (FIG. 14g), as well as in 293, 293shRNA^{Ctl} and 293shRNA^{Slfn} cells (FIG. 14h). In contrast to the pronounced variations in extracellular vRNA, only insignificant differences in intracellular vRNA were evident among those cells. We also analyzed vRNA in the cytoplasmic fraction specifically, as nuclear export of unspliced vRNA is a hallmark of retroviral RNA processing^{6,7,8}, but again found no significant alterations attributable to huSlfn11 (FIG. 21a).

Unlike in BST-2 expressing cells^{9,10} electron-microscopic analysis failed to document accumulation of virions inside or on the surface of virus-producing cells in the presence of huSlfn11 (FIG. 21b). As such, huSlfn11 greatly diminishes the formation of viral particles inside the cell, despite the fact that vRNA is equally available. When the two cell sets were analyzed for the expression of viral proteins encoded by pNL4-3.Luc.R⁺E′, we noted a profound effect of huSlfn11 on p55 Gag and p24 capsid proteins¹¹ (FIGS. 15a and b). To create a clearer picture of the effect of huSlfn11 on p55, we abolished expression of the viral protease by introducing a stop-codon into pro in pNL4-3.Luc.R⁺E⁻. p24 could no longer be detected, however, modulation of p55 expression by huSlfn11 was clearly present (FIG. 22a). As with gag-derived proteins, huSlfn11 had a striking effect on the protein levels of RT, Vif, Vpu and Vpr (FIGS. 15a and b). Surprisingly, we did not observe any reduction of EGFP derived from a co-transfected vector, or of GAPDH (FIGS. 15a and b, bottom), indicating that the limited viral protein production is not due to a global shutdown of protein synthesis.

Even more surprising was that luciferase expression, coded in pNL4-3.Luc.R⁺E⁻ in place of nef, was mostly impervious to huSlfn11 (FIG. 15c, top), contrasting the huSlfn11-mediated diminishment of other proteins encoded on this vector. Strikingly, Nef expression was inhibited by huSlfn11 in the context of pNL4-3-DEnv-EGFP, which has part of env replaced by EGFP, but retains nef in its original position. (FIG. 15c, bottom). Without limiting the invention to any particular mechanism, the inventors conclude that huSlfn11 selectively suppresses viral protein expression via transcript-intrinsic properties rather than external factors or positional elements. This notion is further corroborated by the fact that huSlfn11 had no effect on RRE-mediated events such as nuclear export of unspliced vRNA (FIG. 22b).

### Example 12

Viral genomes display biased nucleotide compositions different from human genes^{12,13,14,15,16,17,18}. Extremely high frequencies of A nucleotides are found in the RNA genomes of lentiviruses and influenza virus^{13,17,19,20}. Wild-isolates of HIV-1, particularly gag and pol sequences, are characterized by low GC content and suboptimal codon usage compared to the host cell preference^{14,21,22,23,24}. The unusual rare codon bias favors A/U in the third position, which induces ribosome pausing and inefficient translation. As the inhibitory effect of huSlfn11 on viral protein expression is intrinsic to the transcripts, we contemplated that huSlfn11 exploits viral codon preferences to specifically attenuate viral protein synthesis. We therefore generated vectors containing only the ORF of HIV-1 gag with either viral codon-bias (Gag^{vir}), or synonymous substitutions optimizing for human cell expression (Gag^{opt}). As evidenced in FIG. 15d, huSlfn11 strongly affected expression of Gag^{vir}, but was without consequence for Gag^{opt} expression. Differences in translation initiation are not likely, as both Gag^{vir} and Gag^{opt} contain the same translation start sequences. Without limiting the invention to any particular mechanism, the inventors are of the view that, this finding strongly suggests that huSlfn11 is exploiting the distinct viral codon bias to selectively attenuate the expression of viral proteins.

### Example 13

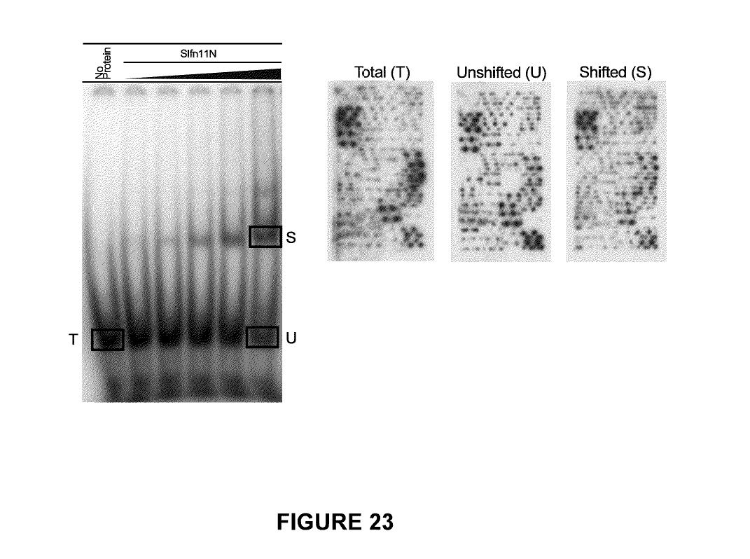

Previous reports indicated changes in cellular tRNA levels after HIV infection²⁰, prompting us to investigate whether huSlfn11 alters the tRNA composition in the absence or presence of HIV. Utilizing tRNA-arrays^{25,26}, we observed little to no changes in tRNA levels as a consequence of huSlfn11 expression (FIG. 16a, left column). However, while HIV triggered substantial changes in tRNA concentrations in huSlfn11-knock-down cells, no changes were observed in the presence of huSlfn11 (FIG. 16a, middle and right). Thus, huSlfn11 counteracts HIV-induced changes in tRNA composition, presumably initiated to promote viral protein synthesis. To test whether huSlfn11 interacts directly with tRNA, we utilized human tRNA as EMSA probe with FPLC-purified His-huSlfn11N. As shown in FIG. 16b, huSlfn11N produced a clear shift of the tRNA in a dose-dependent manner. The shifted band was competed with unlabeled tRNA, but not in-vitro transcribed vRNA (FIG. 16b), and was disrupted by an antibody against huSlfn11 (FIG. 16c). To determine whether huSlfn11 has binding preference for particular tRNAs, we performed EMSAs with increasing amounts of huSlfn11N to obtain ˜50% shifting efficiency (FIG. 23). When the shifted (S), unshifted (U) and total tRNA (T) bands were isolated and recovered tRNAs hybridized to tRNA arrays, no enrichment of particular tRNAs in the shifted band was noticeable (FIG. 23), suggesting a lack of discerning binding preference of huSlfn11N.

### Example 14

The above results led to the premise that any protein that employs similar codon usage as HIV would be modulated by HIV and/or huSlfn11. To unequivocally demonstrate that codon-bias rather than cryptic regulatory elements in gag accounted for the influence of huSlfn11, we tested this hypothesis using non-viral proteins. Natural GFP harbors similar codon-bias as HIV, whereas EGFP has been optimized for mammalian expression by substituting synonymous codons of highly expressed human genes throughout the GFP-ORF²². The data show that co-transfection of pNL4-3.Luc.R⁺E⁻ with V5-tagged GFP and Myc-tagged EGFP in the absence of huSlfn11 resulted in increased GFP, but not EGFP, expression. Most importantly, huSlfn11 inhibited the expression of GFP in a manner identical to viral proteins, whereas EGFP was largely refractory to suppression by huSlfn11 (FIGS. 16d and e). As (E)GFP protein levels have been normalized to their respective mRNA levels (FIG. 16e), and without limiting the invention to any particular mechanism, the inventors conclude that any differences in GFP expression are the consequence of altered protein synthesis rather than variations in transcription or mRNA stability.

### Example 15

Finally, to demonstrate that the anti-retroviral effects of huSlfn11 are not limited to a system employing HIV^{VSV-G} and 293 cells, we utilized CEM cells, a T-cell line widely used to assess HIV replication kinetics. Human PBMCs display similar levels of huSlfn11 after IFNβ stimulation as CEM cells (FIG. 17a). Control or huSlfn11-directed shRNAs were employed to generate stable CEM variants (FIG. 17b), and the resulting CEMshRNA^{Ctl} and CEMshRNA^{Slfn} cells and parental CEM cells were infected with wild-type X4-HIV-1_{LAI}, and viral replication was assessed via p24 ELISA of the supernatants. The data show that CEM and CEMshRNA^{Ctl} cells produced comparable viral titers at all time points. In contrast, CEMshRNA^{Slfn} cells facilitated significantly enhanced HIV-1 replication, yielding an exponentially increasing difference in viral titers (FIG. 17c). A second CEMshRNA^{Slfn} cell line established by means of a distinct huSlfn11 shRNA further corroborated these results (FIG. 24).

In summary, systematic analysis of the HIV replication cycle revealed that huSlfn11 does not inhibit reverse transcription, integration, or production and nuclear export of vRNA, nor did we observe a block in budding or release of viral particles. However, we discovered a selective inhibition in the synthesis of virally encoded proteins. Surprisingly, Coccia et al. had previously observed a specific inhibition of viral protein synthesis in HIV-infected cells in response to interferon, but did not identify the factors responsible for the effect²⁷. We postulate that huSlfn11 acts at the point of virus protein synthesis by exploiting the unique viral codon bias towards A/T nucleotides. This model supports the notion that the antiviral activity of huSlfn11 extends to other viruses with rare codon bias such as influenza, but not to AAV or HSV. The exact mechanism by which HIV alters tRNA function in its favour and how huSlfn11 counteracts this process will require considerable further analysis. Evidently, huSlfn11 interacts with all tRNAs in-vitro. Direct binding of huSlfn11 to tRNA offers the possibility that huSlfn11 either sequesters tRNAs, prevents their maturation via post-transcriptional processing or accelerates their deacylation. In either case, if already rare tRNAs are further reduced, tRNA availability might manifest as the rate limiting step in the synthesis of proteins involving those tRNAs. In contrast, a lesser or no impact would be expected on proteins synthesized via plentiful tRNAs, as even if a fraction of those tRNAs is ‘eliminated’, translation initiation will likely remain the rate limiting event. In conclusion, our results establish huSlfn11 as a potent, interferon-inducible restriction factor against retroviruses such as HIV, mediating its antiviral effects on the basis of codon usage discrimination.

### REFERENCES CITED IN EXAMPLES 1-4 AND FIGS. 1-13

1. Dittmar K A, Mobley E M, Radek A J, Pan T. Exploring the regulation of tRNA distribution on the genomic scale. J Mol Biol. 2004; 337 (1):31-47.
2. Lee M S, Kim Y J. Signaling pathways downstream of pattern-recognition receptors and their cross talk. Annu Rev Biochem. 2007; 76:447-80.
3. Stark G R, Kerr I M, Williams B R, Silverman R H, Schreiber R D. How cells respond to interferons. Annu Rev Biochem. 1998; 67:227-64.
4. Zhou A, Hassel B A, Silverman R H. Expression Cloning of 2-5A-Dependent RNAse: A Uniquely Regulated Mediator of Interferon Action. Cell. 1993; 72:753-5.
5. Katze M G, Wambach M, Wong M-L, Garfinkle M, Meurs E, Chong K, et al. Functional Expression and RNA Binding Analysis of the Interferon-Induced, Double-Stranded RNA-Activated, 68,000-Mᵣ Protein Kinase in a Cell-Free System. Molecular Cellular Biology. 1991; 11 (11):5497-505.
6. Marotta G, et al. Induction of LFA-1/CD11a and ICAM-1/CD54 adhesion molecules on neoplastic B cells during in vivo treatment of chronic lymphocytic leukemia with interferon-alpha 2. Blood. 1993; 81 (1):267-9. No abstract available.
7. Loh J E, Chang C-H, Fodor W L, Flavell R A. Dissection of the interferon-g-MHC class II signal transduction pathway reveals that type 1 and type 11 interferon systems share common signalling components. EMBO. 1992; 11 (4):1351-63.
8. Schwarz D A, Katayama C D, Hedrick S M. Schlafen, a new family of growth regulatory genes that affect thymocyte development. Immunity. 1998; 9 (5):657-68.
9. Geserick P, Kaiser F, Klemm U, Kaufmann S H, Zerrahn J. Modulation of T cell development and activation by novel members of the Schlafen (slfn) gene family harbouring an RNA helicase-like motif. Int Immunol. 2004; 16 (10):1535-48.
10. Brady G, Boggan L, Bowie A, O'Neill L A. Schlafen-1 causes a cell cycle arrest by inhibiting induction of cyclin D1. J Biol Chem. 2005; 280 (35):30723-34.
11. Eskra L, Mathison A, Splitter G. Microarray analysis of mRNA levels from RAW264.7 macrophages infected with Brucella abortus. Infect Immun. 2003; 71 (3):1125-33.
12. Huthoff H, Towers G J. Restriction of retroviral replication by APOBEC3G/F and TRIM5alpha. Trends Microbiol. 2008; 16 (12):612-9.
13. Sakuma R, Mael A A, Ikeda Y. Alpha interferon enhances TRIM5alpha-mediated antiviral activities in human and rhesus monkey cells. J Virol. 2007; 81 (18):10201-6. PMCID: PMC2045407.
14. Sayah D M, Sokolskaja E, Berthoux L, Luban J. Cyclophilin A retrotransposition into TRIM5 explains owl monkey resistance to HIV-1. Nature. 2004; 430 (6999):569-73.
15. Stremlau M, Owens C M, Perron M J, Kiessling M, Autissier P, Sodroski J. The cytoplasmic body component TRIM5alpha restricts HIV-1 infection in Old World monkeys. Nature. 2004; 427 (6977):848-53.
16. Barr S D, Smiley J R, Bushman F D. The interferon response inhibits HIV particle production by induction of TRIM22. PLoS Pathog. 2008; 4 (2):e1000007. PMCID: PMC2279259.
17. Uchil P D, Quinlan B D, Chan W T, Luna J M, Mothes W. TRIM E3 ligases interfere with early and late stages of the retroviral life cycle. PLoS Pathog. 2008; 4 (2):e16. PMCID: PMC2222954.
18. Carthagena L, Bergamaschi A, Luna J M, David A, Uchil P D, Margottin-Goguet F, et al. Human TRIM gene expression in response to interferons. PLoS One. 2009; 4 (3):e4894. PMCID: PMC2654144.
19. Bonvin M, Achermann F, Greeve I, Stroka D, Keogh A, Inderbitzin D, et al. Interferon-inducible expression of APOBEC3 editing enzymes in human hepatocytes and inhibition of hepatitis B virus replication. Hepatology. 2006; 43 (6):1364-74.
20. Chen K, Huang J, Zhang C, Huang S, Nunnari G, Wang F X, et al. Alpha interferon potently enhances the anti-human immunodeficiency virus type 1 activity of APOBEC3G in resting primary CD4 T cells. J Virol. 2006; 80 (15):7645-57. PMCID: PMC1563726.
21. Goila-Gaur R, Strebel K. HIV-1 Vif, APOBEC, and intrinsic immunity. Retrovirology. 2008; 5:51. PMCID: PMC2443170.
22. Peng G, Lei K J, Jin W, Greenwell-Wild T, Wahl S M. Induction of APOBEC3 family proteins, a defensive maneuver underlying interferon-induced anti-HIV-1 activity. J Exp Med. 2006; 203 (1):41-6. PMCID: PMC2118075.
23. Sheehy A M, Gaddis N C, Choi J D, Malim M H. Isolation of a human gene that inhibits HIV-1 infection and is suppressed by the viral Vif protein. Nature. 2002; 418 (6898):646-50.
24. Neil S J, Zang T, Bieniasz P D. Tetherin inhibits retrovirus release and is antagonized by HIV-1 Vpu. Nature. 2008; 451 (7177):425-30.
25. Van Damme N, Goff D, Katsura C, Jorgenson R L, Mitchell R, Johnson M C, et al. The interferon-induced protein BST-2 restricts HIV-1 release and is downregulated from the cell surface by the viral Vpu protein. Cell Host Microbe. 2008; 3 (4):245-52. PMCID: PMC2474773.
26. Okumura A, Lu G, Pitha-Rowe I, Pitha P M. Innate antiviral response targets HIV-1 release by the induction of ubiquitin-like protein ISG15. Proc Natl Acad Sci USA. 2006; 103 (5):1440-5. PMCID: PMC1360585.
27. Espert L, Degols G, Lin Y L, Vincent T, Benkirane M, Mechti N. Interferon-induced exonuclease ISG20 exhibits an antiviral activity against human immunodeficiency virus type 1. J Gen Virol. 2005; 86 (Pt 8):2221-9.
28. Haase A T. Perils at mucosal front lines for HIV and SIV and their hosts. Nat Rev Immunol. 2005; 5 (10):783-92.
29. Zhao L, Neumann B, Murphy K, Silke J, Gonda T J. Lack of reproducible growth inhibition by Schlafen1 and Schlafen2 in vitro. Blood Cells Mol Dis. 2008; 41 (2):188-93.
30. Coleman J R, Papamichail D, Skiena S, Futcher B, Wimmer E, Mueller S. Virus attenuation by genome-scale changes in codon pair bias. Science. 2008; 320 (5884):1784-7. PMCID: PMC2754401.
31. Zhong J, Li Y, Zhao S, Liu S, Zhang Z. Mutation pressure shapes codon usage in the G C-Rich genome of foot-and-mouth disease virus. Virus Genes. 2007; 35 (3):767-76.
32. Pinto R M, Aragones L, Costafreda M I, Ribes E, Bosch A. Codon usage and replicative strategies of hepatitis A virus. Virus Res. 2007; 127 (2):158-63.
33. Zhou T, Gu W, Ma J, Sun X, Lu Z. Analysis of synonymous codon usage in H5N1 virus and other influenza A viruses. Biosystems. 2005; 81 (1):77-86.
34. Meintjes P L, Rodrigo A G. Evolution of relative synonymous codon usage in Human Immunodeficiency Virus type-1. J Bioinform Comput Biol. 2005; 3 (1):157-68.
35. Frelin L, Ahlen G, Alheim M, Weiland O, Barnfield C, Liljestrom P, et al. Codon optimization and mRNA amplification effectively enhances the immunogenicity of the hepatitis C virus nonstructural 3/4A gene. Gene Ther. 2004; 11 (6):522-33.
36. Forsberg R, Christiansen F B. A codon-based model of host-specific selection in parasites, with an application to the influenza A virus. Mol Biol Evol. 2003; 20 (8):1252-9.
37. Plotkin J B, Dushoff J. Codon bias and frequency-dependent selection on the hemagglutinin epitopes of influenza A virus. Proc Natl Acad Sci USA. 2003; 100 (12):7152-7. PMCID: PMC165845.
38. Grantham P, Perrin P. AIDS virus and HTLV-I differ in codon choices. Nature. 1986; 319 (6056):727-8.
39. Hruby D E, Guarino L A. Novel codon utilization within the vaccinia virus thymidine kinase gene. Virus Res. 1984; 1 (4):315-20.
40. Wong E H, Smith D K, Rabadan R, Peiris M, Poon L L. Codon usage bias and the evolution of influenza A viruses. Codon Usage Biases of Influenza Virus. BMC Evol Biol. 10:253. PMCID: PMC2933640.
41. van Weringh A, Ragonnet-Cronin M, Pranckeviciene E, Pavon-Eternod M, Kleiman L, Xia X. HIV-1 modulates the tRNA pool to improve translation efficiency. Mol Biol Evol.
42. Kofman A, Graf M, Bojak A, Deml L, Bieler K, Kharazova A, et al. HIV-1 gag expression is quantitatively dependent on the ratio of native and optimized codons. Tsitologiia. 2003; 45 (1):86-93.
43. Haas J, Park E C, Seed B. Codon usage limitation in the expression of HIV-1 envelope glycoprotein. Curr Biol. 1996; 6 (3):315-24.
44. Berkhout B, van Hemert F J. The unusual nucleotide content of the HIV RNA genome results in a biased amino acid composition of HIV proteins. Nucleic Acids Res. 1994; 22 (9):1705-11. PMCID: PMC308053.
45. Kypr J, Mrazek J. Unusual codon usage of HIV. Nature. 1987; 327 (6117):20.
46. van Weringh A, Ragonnet-Cronin M, Pranckeviciene E, Pavon-Eternod M, Kleiman L, Xia X. HIV-1 modulates the tRNA pool to improve translation efficiency. Mol Biol Evol. 28 (6):1827-34. PMCID: PMC3098512.
47. Erzberger J P, Berger J M. Evolutionary relationships and structural mechanisms of AAA+ proteins. Annu Rev Biophys Biomol Struct. 2006; 35:93-114.
48. Alexandrov A, Chemyakov I, Gu W, Hiley S L, Hughes T R, Grayhack E J, et al. Rapid tRNA decay can result from lack of nonessential modifications. Mol Cell 2006; 21 (1):87-96.
49. Carthagena L, Parise M C, Ringeard M, Chelbi-Alix M K, Hazan U, Nisole S. Implication of TRIM alpha and TRIMCyp in interferon-induced anti-retroviral restriction activities. Retrovirology. 2008; 5:59. PMCID: PMC2483995.
50. Afonso C L, Tulman E R, Lu Z, Zsak L, Sandybaev N T, Kerembekova U Z, et al. The genome of camelpox virus. Virology. 2002; 295 (1):1-9.
51. Bustos O, Naik S, Ayers G, Casola C, Perez-Lamigueiro M A, Chippindale P T, et al. Evolution of the Schlafen genes, a gene family associated with embryonic lethality, meiotic drive, immune processes and orthopoxvirus virulence. Gene. 2009; 447 (1):1-11.
52. Bannert N, Kurth R. Retroelements and the human genome: new perspectives on an old relation. Proc Natl Acad Sci USA. 2004; 101 Suppl 2:14572-9. PMCID: PMC521986.

### REFERENCES CITED IN EXAMPLES 5-15 AND FIGS. 14-24

¹ Sohn, W. J. et al. Novel transcriptional regulation of the schlafen-2 gene in macrophages in response to TLR-triggered stimulation. Mol Immunol 44, 3273-3282 (2007).
² Yoneyama, M. et al. Shared and unique functions of the DExD/H-box helicases RIG-I, MDA5, and LGP2 in antiviral innate immunity. J Immunol 175, 2851-2858, doi:175/5/2851 (pii) (2005).
³ Schwarz, D. A., Katayama, C. D. & Hedrick, S. M. Schlafen, a new family of growth regulatory genes that affect thymocyte development. Immunity 9, 657-668, doi:S1074-7613(00)80663-9 (pii) (1998).
⁴ Geserick, P., Kaiser, F., Klemm, U., Kaufmann, S. H. & Zerrahn, J. Modulation of T cell development and activation by novel members of the Schlafen (slfn) gene family harbouring an RNA helicase-like motif. Int Immunol 16, 1535-1548 (2004).
⁵ Berger, M. et al. An Slfn2 mutation causes lymphoid and myeloid immunodeficiency due to loss of immune cell quiescence. Nat Immunol 11, 335-343, doi:ni.1847 (pii) 10.1038/ni.1847 (2010).
⁶ Fritz, C. C. & Green, M. R. HIV Rev uses a conserved cellular protein export pathway for the nucleocytoplasmic transport of viral RNAs. Curr Biol 6, 848-854, doi:S0960-9822 (02)00608-5 (pii) (1996).
⁷ Fischer, U., Huber, J., Boelens, W. C., Mattaj, I. W. & Luehrmann, R. The HIV-1 Rev Activation Domain is a Nuclear Export Signal that Accesses an Export Pathway used by Specific Cellular RNAs. Cell 82, 475-483 (1995).
⁸ Malim, M. H., Hauber, J., Le, S. Y., Maizel, J. V. & Cullen, B. R. The HIV-1 rev trans-activator acts through a structured target sequence to activate nuclear export of unspliced viral mRNA. Nature 338, 254-257, doi:10.1038/338254a0 (1989).
⁹ Van Damme, N. et al. The interferon-induced protein BST-2 restricts HIV-1 release and is downregulated from the cell surface by the viral Vpu protein. Cell Host Microbe 3, 245-252, doi:S1931-3128(08)00086-3 (pii) 10.1016/j.chom.2008.03.001 (2008).
¹⁰ Neil, S. J., Zang, T. & Bieniasz, P. D. Tetherin inhibits retrovirus release and is antagonized by HIV-1 Vpu. Nature 451, 425-430, doi:nature06553 (pii) 10.1038/nature06553 (2008).
¹¹ Ganser-Pornillos, B. K., Yeager, M. & Sundquist, W. I. The structural biology of HIV assembly. Curr Opin Struct Biol 18, 203-217, doi:S0959-440X(08)00028-6 (pii) 10.1016/j.sbi.2008.02.001 (2008).
¹² Coleman, J. R. et al. Virus attenuation by genome-scale changes in codon pair bias. Science 320, 1784-1787, doi:320/5884/1784 (pii) 10.1126/science.1155761 (2008).
¹³ Zhou, T., Gu, W., Ma, J., Sun, X. & Lu, Z. Analysis of synonymous codon usage in H5N1 virus and other influenza A viruses. Biosystems 81, 77-86, doi:S0303-2647(05)00022-5 (pii) 10.1016/j.biosystems.2005.03.002 (2005).
¹⁴ Meintjes, P. L. & Rodrigo, A. G. Evolution of relative synonymous codon usage in Human Immunodeficiency Virus type-1. J Bioinform Comput Biol 3, 157-168, doi:S0219720005000953 (pii) (2005).
¹⁵ Frelin, L. et al. Codon optimization and mRNA amplification effectively enhances the immunogenicity of the hepatitis C virus nonstructural 3/4A gene. Gene Ther 11, 522-533, doi:10.1038/sj.gt.3302184 3302184 (pii) (2004).
¹⁶ Forsberg, R. & Christiansen, F. B. A codon-based model of host-specific selection in parasites, with an application to the influenza A virus. Mol Biol Evol 20, 1252-1259, doi:10.1093/molbev/msg149 msg149 (pii) (2003).
¹⁷ Plotkin, J. B. & Dushoff, J. Codon bias and frequency-dependent selection on the hemagglutinin epitopes of influenza A virus. Proc Natl Acad Sci USA 100, 7152-7157, doi:10.1073/pnas.1132114100 1132114100 (pii) (2003).
¹⁸ Grantham, P. & Perrin, P. AIDS virus and HTLV-I differ in codon choices. Nature 319, 727-728, doi:10.1038/319727b0 (1986).
¹⁹ Wong, E. H., Smith, D. K., Rabadan, R., Peiris, M. & Poon, L. L. Codon usage bias and the evolution of influenza A viruses. Codon Usage Biases of Influenza Virus. BMC Evol Biol 10, 253, doi:1471-2148-10-253 (pii) 10.1186/1471-2148-10-253.
²⁰ van Weringh, A. et al. HIV-1 modulates the tRNA pool to improve translation efficiency. Mol Biol Evol, doi:msr005 (pii) 10.1093/molbev/msr005 (2011).
²¹ Kofman, A. et al. HIV-1 gag expression is quantitatively dependent on the ratio of native and optimized codons. Tsitologiia 45, 86-93 (2003).
²² Haas, J., Park, E. C. & Seed, B. Codon usage limitation in the expression of HIV-1 envelope glycoprotein. Curr Biol 6, 315-324, doi:S0960-9822(02)00482-7 (pii) (1996).
²³ Berkhout, B. & van Hemert, F. J. The unusual nucleotide content of the HIV RNA genome results in a biased amino acid composition of HIV proteins. Nucleic Acids Res 22, 1705-1711 (1994).
²⁴ Kypr, J. & Mrazek, J. Unusual codon usage of HIV. Nature 327, 20, doi:10.1038/327020a0 (1987).
²⁵ Pavon-Eternod, M. et al. tRNA over-expression in breast cancer and functional consequences. Nucleic Acids Res 37, 7268-7280, doi:gkp787 (pii) 10.1093/nar/gkp787 (2009).
²⁶ Dittmar, K. A., Mobley, E. M., Radek, A. J. & Pan, T. Exploring the regulation of tRNA distribution on the genomic scale. J Mol Biol 337, 31-47, doi:10.1016/j.jmb.2004.01.024 S0022283604000828 (pii) (2004).
²⁷ Coccia, E. M., Krust, B. & Hovanessian, A. G. Specific inhibition of viral protein synthesis in HIV-infected cells in response to interferon treatment. J Biol Chem 269, 23087-23094 (1994).
²⁸ Connor, R. I., Chen, B. K., Choe, S. & Landau, N. R. Vpr is required for efficient replication of human immunodeficiency virus type-1 in mononuclear phagocytes. Virology 206, 935-944, doi:S0042-6822(85)71016-1 (pii) 10.1006/viro.1995.1016 (1995).
   All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiment, it should be understood that the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A method for screening one or more candidate compounds for activity in altering the level of expression of Schlafen (Slfn) in a mammalian target cell, comprising
a) contacting said one or more candidate compounds with a mammalian target cell that expresses Schlafen (Slfn) to produce a contacted cell, and
b) measuring the level of expression of one or more Slfn in said contacted cell and the uncontacted target cell.

2. The method of claim 1, wherein an altered level of expression of one or more Slfn in said contacted cell compared to in said uncontacted target cell identifies said one or more candidate compounds as having activity in altering the level of expression of Slfn in said uncontacted target cell.

3. A method for screening one or more one or more candidate compounds for activity in altering the level of virus infection of a mammalian host cell, comprising
a) providing
i) a virus,
ii) a mammalian host cell that is susceptible to said virus, and
iii) said one or more candidate compounds,
b) contacting said mammalian host cell with said virus and with said one or more candidate compounds under conditions for infection of said mammalian host cell with said virus, to produce a test contacted cell,
c) contacting said mammalian host cell with said virus, in the absence of said one or more candidate compounds, under conditions for infection of said transgenic mammalian cell with said virus, to produce a control contacted cell, and
d) measuring the level of virus infection of said test contacted cell and of said control contacted host cell.

4. The method of claim 3, wherein an altered level of infection of said contacted test cell compared to said control contacted cell identifies said one or more candidate compounds as having activity in altering the level of virus infection of said host cell.

5. The method of claim 3, further comprising detecting a reduction of from 20% to 100% in the level of said virus infection of said test contacted cell compared to said control contacted cell.

6. The method of claim 3, further comprising measuring the level of expression of one or more Slfn in one or more of said host cell, said test contacted cell, and said control contacted cell.

7. A method for screening one or more candidate compounds for altering the level of expression of one or more virus proteins, comprising
a) providing
i) a first cell that expresses a first heterologous nucleotide sequence encoding a heterologous polypeptide, wherein said first heterologous nucleotide sequence is optimized for gene expression based on codon bias of said virus,
ii) a second cell that expresses a second heterologous nucleotide sequence encoding said heterologous polypeptide, wherein said second heterologous nucleotide sequence is optimized for gene expression based on codon bias of a cell that is permissive to said virus, and
iii) said one or more candidate compounds,
b) contacting said one or more candidate compounds with said first cell to produce a first contacted cell,
c) contacting said one or more candidate compounds with said second cell to produce a second contacted cell, and
d) measuring the level of expression of said heterologous polypeptide in said first contacted cell and in said second contacted cell.

8. The method of claim 7, wherein measuring an altered level of expression of said heterologous polypeptide in said first contacted cell and measuring a substantially unaltered level of expression of said heterologous polypeptide in said second contacted cell identifies said one or more candidate compounds as specifically altering the level of expression of one or more proteins of said virus.

9. The method of claim 7, wherein said one or more candidate compounds comprise a test polypeptide sequence that is encoded by a nucleic acid sequence comprised in said first cell and in said second cell, and said contacting step comprises expressing said test polypeptide sequence by said first cell and by said second cell.

10. The method of claim 9, wherein said test polypeptide sequence comprises Schlafen (Slfn).

11. A method for identifying one or more Schlafen (Slfn) polymorphisms as altering the level of risk of a subject to infection by a virus, comprising
a) providing
i) a first sample comprising genomic Schlafen (Slfn) sequence from a first population of subjects infected by said virus, and
ii) a second sample comprising genomic Schlafen (Slfn) sequence from a second population of subjects not infected by said virus,
b) determining the presence of
i) wild type Schlafen (Slfn) gene sequence in said first sample and in said second sample, and
ii) one or more Schlafen (Slfn) polymorphisms in said first sample and in said second sample,
c) measuring the ratio of said one or more Schlafen (Slfn) polymorphisms relative to said wild type Schlafen (Slfn) gene sequence in
i) said first sample, and
ii) in said second sample,
wherein measuring a difference in said ratio in said first sample relative to said ratio in said second sample identifies said one or more Schlafen (Slfn) polymorphisms as altering the level of risk of said mammalian subject to infection by said virus.

12. A method for determining the risk of a mammalian subject to infection by a virus, comprising detecting, in a biological sample that comprises genomic Schlafen (Slfn) sequence from said subject, one or more Schlafen (Slfn) polymorphisms that is identified by the method of claim 11.

13. The method of claim 12, wherein said detecting comprises detecting an increased ratio of said one or more Schlafen (Slfn) polymorphisms in said subject.

14. A method for identifying one or more Schlafen (Slfn) as altering the level of risk of a subject to infection by a virus, comprising measuring the level of expression of one or more Schlafen (Slfn) in
i) a first sample from a first population of subjects infected by said virus, and
ii) a second sample from a second population of subjects not infected by said virus,
wherein measuring an altered level of expression in said first sample relative to said second sample, identifies said one or more Schlafen (Slfn) as altering the level of risk of said subject to infection by said virus.

15. A method for determining the risk of a mammalian subject to infection by a virus, comprising detecting in a biological sample from said subject an altered level of expression of one or more Schlafen (Slfn) identified by the method of claim 14.

16. A method for altering the level of expression of one or more virus protein by a target mammalian cell that is susceptible to a virus, comprising administering to said cell one or more compounds in an amount that alters the level of expression of one or more Schlafen (Slfn) in said cell.

17. The method of claim 16, further comprising measuring the level of expression of said one or more Schlafen (Slfn) in said cell.

18. The method of claim 16, wherein said one or more compounds that alter the level of expression of said Slfn in said target mammalian cell is identified by the method of claim 1.

19. The method of claim 16, wherein said cell is in vivo in a mammalian subject.

20. The method of claim 16, further comprising measuring the level of expression of said one or more virus proteins.

21. A transgenic non-human mammal comprising a homozygous deletion of at least a portion of one or more Schlafen (Slfn) gene, wherein said deletion is comprised in one or more of CD4+ T cell, macrophage cell, and dendritic cell of said mammal.

22. The transgenic mammal of claim 21, wherein said at least a portion encodes from amino acid 1 to 579 of a Slfn protein.

23. The transgenic mammal of claim 21, wherein said at least a portion encodes a polypeptide sequence that is at least 50-amino acids long and that is comprised by the sequence from amino acid 1 to 579 of a Slfn protein.

24. The transgenic mammal of claim 21, wherein said transgenic non-human mammal is susceptible to infection by human immunodeficiency virus (HIV).

25. The transgenic mammal of claim 21, wherein said transgenic non-human mammal exhibits one or more symptoms of Acquired Immune Deficiency Syndrome (AIDS).

26. A method for screening one or more one or more candidate compounds for activity in altering the level of infection of a mammalian host cell by human immunodeficiency virus (HIV), comprising
a) providing
i) human immunodeficiency virus (HIV),
ii) the transgenic non-human mammal of claim 21, and
iii) said one or more candidate compounds,
b) contacting said transgenic non-human mammal with said HIV and with said one or more candidate compounds under conditions for infection of cells of said mammal with said HIV to produce a contacted test mammal,
c) contacting said transgenic non-human mammal with said HIV, in the absence of said one or more candidate compounds, under conditions for infection of cells of said mammal with said HIV to produce a contacted control mammal, and
d) measuring the level of HIV infection of cells of said contacted test mammal and of cells of said contacted control mammal,
wherein measuring an altered level of HIV infection of said cells of said contacted test mammal compared to said cells of said contacted control mammal identifies said one or more candidate compounds as having activity in altering the level of said HIV infection of said host cell.

27. A transgenic non-human mammalian cell comprising a deletion of Schlafen (Slfn) gene.

28. A method for screening one or more one or more candidate compounds for activity in altering the level of infection of a mammalian cell by a virus, comprising
a) providing
i) virus,
ii) the transgenic mammalian cell of claim 27, and
iii) said one or more candidate compounds,
b) contacting said transgenic mammalian cell with said virus and with said one or more candidate compounds under conditions for infection of said transgenic mammalian cell with said virus to produce a contacted test cell,
c) contacting said transgenic mammalian cell with said virus, in the absence of said one or more candidate compounds, under conditions for infection of said transgenic mammalian cell with said virus to produce a contacted control cell, and
d) measuring the level of infection of said contacted test cell and of said contacted control cell with said virus,
wherein measuring an altered level of virus infection of said contacted test cell compared to said contacted control cell identifies said one or more candidate compounds as having activity in altering the level of infection of said mammalian cell by said virus.

29. The method of claim 28, wherein said one or more candidate compounds are identified by the method of claim 1.
